(19) United States
(12) Patent Application Publication
(10) Pub. No.: US 2023/0078372 A1
(43) Pub. Date: Mar. 16, 2023

(54) Prosthetic Atrio-Ventricular Valve Systems and Devices

(71) Applicant: Laguna Tech USA, Inc., Irvine, CA (US)

(72) Inventors: Gilbert Madrid, Dana Point, CA (US); Wei Wang, Garden Grove, CA (US)

(21) Appl. No.: 17/930,691

(22) Filed: Sep. 8, 2022

Related U.S. Application Data
(60) Provisional application No. 63/261,256, filed on Sep. 15, 2021.

(51) Int. Cl. A61F 2/24

(52) U.S. Cl. CPC A61F 2/2418; A61F 2220/0008; A61F 2220/0075; A61F 2230/0054; A61F 2210/0014

(57) ABSTRACT

A prosthetic mitral valve system that comprises a valve dock and a prosthetic mitral valve is disclosed. The valve dock comprises clamp jaws that sandwich the native mitral valve leaflets and the native mitral valve annulus between them anchoring the prosthetic mitral valve system at or adjacent to the native mitral valve annulus. Further, a prosthetic mitral valve comprising atrial and ventricular clamp jaws and which can be implanted at or adjacent to the native mitral valve annulus without a valve dock system is disclosed. Novel methods and systems for treating mitral valve disease or malfunction by percutaneous replacement of the mitral valve (or the tricuspid valve) are disclosed.

### CROSS REFERENCE TO RELATED APPLICATION(S)

The present application claims the benefit of the filing date of U.S. Provisional Application No. 63/261,256, filed Sep. 15, 2021, the entire disclosure of which is incorporated herein by reference.

### FIELD OF THE INVENTION

The present invention relates generally to prosthetic heart valve devices. In particular, some embodiments relate to prosthetic mitral valves and methods and devices for transcatheter or transapical repair and/or replacement of native mitral valves using prosthetic heart valves and other devices.

### BACKGROUND OF THE INVENTION

Mitral valve function can be adversely affected by phenomenon, such as, mitral valve regurgitation, mitral valve prolapse and mitral valve stenosis. Mitral valve regurgitation, which can be degenerative or functional, is one of the most prevalent valvulopathies worldwide. It is a disorder of the heart in which the leaflets of the mitral valve fail to coapt into apposition at peak contraction pressures, resulting in abnormal leaking of blood from the left ventricle into the left atrium (in a normally functioning heart, blood flows from the left atrium to the left ventricle, with the mitral valve acting as a check valve to prevent blood flow in the opposite direction). There are a number of factors that may affect the proper closure of the mitral valve leaflets. For example, dilation of the heart muscle may result in an enlarged mitral annulus, which makes it difficult for the mitral valve leaflets to coapt during systole. A stretch or tear in the chordae tendineae, the tendons connecting the papillary muscles to the inferior side of the mitral valve leaflets, may also affect proper closure of the mitral annulus. A ruptured chordae tendineae, for example, may cause a valve leaflet to prolapse into the left atrium due to inadequate tension on the leaflet. Abnormal backflow can also occur when the functioning of the papillary muscles is compromised, for example, due to ischemia. As the left ventricle contracts during systole, the affected papillary muscles do not contract sufficiently to effect proper closure.

Mitral valve prolapse, or when the mitral leaflets bulge abnormally up in to the left atrium, causes irregular behavior of the mitral valve and may also lead to mitral valve regurgitation. Normal functioning of the mitral valve may also be affected by mitral valve stenosis, or a narrowing of the mitral valve orifice, which—impedes the filling of the left ventricle during diastole.

Mitral valve disease or malfunction, such as described above, is often treated using surgical procedures for repair or replacement of the valve. However, such repair and replacement procedures have the disadvantage of lack of durability of the devices or improper sizing of annuloplasty rings or replacement valves, leading to unsatisfactory outcomes for the patient. Further, many of the repair procedures are highly dependent upon the skill of the cardiac surgeon. Moreover, surgical treatment is not feasible in every case. The elderly and frail with several comorbidities and left ventricular dysfunction have to be managed conservatively. Accordingly, there is a need for percutaneous treatment of an affected mitral valve for those patients who present a high risk for surgery, similar to percutaneous treatment of aortic valve disease.

Transcatheter aortic valve replacement (TAVR) is now being successfully performed using percutaneous prosthetic valves, such as, the CoreValve Revalving® System from Medtronic/Corevalve Inc. (Irvine, Calif., USA) and the Edwards-Sapien® Valve from Edwards Lifesciences (Irvine, Calif., USA). Both valve systems include an expandable frame housing a tri-leaflet bioprosthetic valve. The frame is expanded to fit the largely symmetric, circular and rigid aortic annulus.

Mitral valve replacement, compared with aortic valve replacement, poses unique anatomical obstacles, rendering percutaneous mitral valve replacement significantly more challenging than aortic valve replacement. Thus, in contrast to TAVR, transcatheter mitral valve replacement (TMVR) is a much more complex procedure because of the mitral valve's anatomy and shape, lack of calcification, and its relationship with adjacent structures. The mitral valve apparatus is mainly composed of the mitral annulus (AN), two leaflets (LF), left atrium (LA), left ventricle (LV), papillary muscles (PM), and tendinous chords (CT). Any disturbance of these components may lead to mitral valve dysfunction. The annulus's D shape with 3D saddle-shaped geometry and size changes with each cardiac cycle pose unique challenges in designing a viable TMVR. Additionally, the lack of calcification and the lack of significant amount of radial support from surrounding tissue makes anchoring of a TMVR difficult and, therefore, TMVR designs must provide for a robust anchoring system. The aortic valve, on the other hand, is completely surrounded by fibro-elastic tissue or calcified structures, helping to anchor a prosthetic valve by providing native structural support. The mitral valve, on the other hand, is bound by muscular tissue on the outer wall only. The inner wall of the mitral valve is bound by a thin vessel wall separating the mitral valve annulus from the inferior portion of the aortic outflow tract. As a result, significant radial forces on the mitral annulus, such as those imparted by expanding stent prostheses, could lead to collapse of the inferior portion of the aortic tract with potentially fatal consequences.

The chordae tendineae of the left ventricle is also thought to present an obstacle in deploying a mitral valve prosthesis. This is unique to the mitral valve since aortic valve anatomy does not include chordae. The maze of chordae in the left ventricle makes navigating and positioning a deployment catheter that much more difficult in mitral valve replacement and repair.

Given the unique challenges of mitral valve replacement, an adequate pre-procedural study is near mandatory, and comprises multimodality imaging to define mitral regurgitation, to evaluate a patient's eligibility according to anatomic characteristics, to plan access for implantation, and to identify issues that could potentially affect TMVR. There are several challenges such as mitral valve position, valve sealing, the proximity of the left ventricle outflow tract (LVOT), delivery system size, prosthesis anchoring, and valve thrombogenicity. Importantly, the current generation of prosthetic mitral valves used for TMVR are such that many patients evaluated during such pre-procedural study are rejected for TMVR. Therefore, there is need for an improved prosthetic mitral valve system that better responds to the challenges posed by the anatomy of the mitral valve and associated heart structures.

The triscuspid valve on the right side of the heart, although it normally has three leaflets, poses similar challenges to less invasive treatment as the mitral valve. Therefore, there is a need for a better prosthesis to treat tricuspid valve disease as well.

Given the difficulties associated with current procedures, there remains the need for simple, effective, and less invasive devices and methods for treating diseased or malfunctioning atrio-ventricular heart valves.

### SUMMARY OF THE INVENTION

Assemblies, devices and methods are set forth herein for percutaneous replacement of native atrio-ventricular heart valves, for example, native mitral valves. Several of the details set forth below are provided to describe the following examples and methods in a manner sufficient to enable a person skilled in the relevant art to practice, make and use them. Several of the details and advantages described below, however, may not be necessary to practice certain examples and methods of the technology. Additionally, the technology may include other examples and methods that are within the scope of the claims but are not described in detail.

Embodiments of the instant technology provide systems, methods and devices to treat valves of the body, such as heart valves including the mitral valve. The apparatus and methods enable a percutaneous approach using a catheter delivered intravascularly through a vein or artery into the heart. Additionally, the apparatus and methods enable other less-invasive approaches including trans-apical, trans-atrial, and direct aortic delivery of a prosthetic replacement valve to a target location in the heart. The apparatus and methods enable a prosthetic device to be anchored at a native valve location by engagement with structures such as the native mitral valve annulus and/or native mitral valve leaflets. Additionally, the embodiments of the devices and methods as described herein can be combined with many known surgeries and procedures, such as known methods of accessing the valves of the heart (e.g., the mitral valve or triscuspid valve) with antegrade or retrograde approaches, and combinations thereof.

Some embodiments of the instant technology are directed to a prosthetic mitral valve for implantation at a native mitral valve of a heart having a left atrium and a left ventricle, the native mitral valve having a native mitral valve annulus and native mitral valve leaflets, wherein the native mitral valve annulus has an atrium side that faces the left atrium of the heart and a ventricle side that faces the left ventricle of the heart, the prosthetic mitral valve system comprising: an valve stent having an inflow end and an outflow end, one or more atrial clamp jaws projecting radially outwards from the valve stent, one or more ventricular clamp jaws projecting radially outwards from the valve stent, and a plurality of prosthetic leaflets coupled to the valve stent at commissure attachment features of the valve stent, wherein when the prosthetic mitral valve is deployed at the site of the native mitral valve, the ventricular clamp jaws are deployed on the ventricle side of the native mitral valve annulus and atrial clamp jaws are deployed on the atrial side of the native mitral valve annulus such that the atrial clamp jaws and ventricular clamp jaws are sufficiently resiliently biased with respect to each other to grip the native mitral valve leaflets and the native mitral valve annulus between them.

In some embodiments, the valve stent of the prosthetic mitral valve set forth above is comprised of a shape memory alloy. Further, in some embodiments, the valve stent has a deployed configuration and a shape set configuration such that at least a portion of the ventricular clamp jaws is more distal to the outflow end in said shape set configuration than in the deployed configuration.

In some embodiments, the prosthetic mitral valve has one or more ventricular clamp jaws that are atraumatic. In other embodiments, the atrial clamp jaws of the prosthetic mitral valve are atraumatic, and in yet other embodiments, both the atrial and ventricular clamp jaws are atraumatic. In some embodiments of the prosthetic mitral valve, one or more of the atrial and/or ventricular clamp jaws may be covered by fabric, and in some embodiments, the fabric may cover one side of the atrial and/or ventricular clamp jaws.

In some embodiments of the prosthetic mitral valve, one of more removeable suture loops having free ends is connected to one or more separate atrial clamp jaws, wherein the removeable suture loops can be used to adjust the placement of the prosthetic mitral valve at the native mitral valve annulus post implantation and can be removed after the desired placement has been achieved. In some embodiments, three removeable suture loops are connected to three separate atrial clamp jaws approximately 120 degrees apart from each other.

In some embodiments, the prosthetic mitral valve further comprises

a circumferential suture that is connected to the atrial clamp jaws along the circumference of the prosthetic mitral valve. In such embodiments, the removeable suture loops for adjusting the placement of the valve are connected to the circumferential suture.

Some embodiments of the instant technology are directed to a prosthetic mitral valve for implantation at a native mitral valve of a heart having a left atrium and a left ventricle, the native mitral valve having a native mitral valve annulus and native mitral valve leaflets, wherein the native mitral valve annulus has an atrium side that faces the left atrium of the heart and a ventricle side that faces the left ventricle of the heart, the prosthetic mitral valve system comprising an expandable atrial valve stent having an inflow end and an outside diameter, one or more atrial clamp jaws projecting radially outwards from the expandable atrial valve stent, an expandable ventricular valve stent having an outflow end and an inside diameter that is greater than the outside diameter of the expandable atrial valve stent, one or more ventricular clamp jaws projecting radially outwards from the valve stent, and a plurality of prosthetic leaflets coupled to the expandable atrial valve stent at commissure attachment features of the expandable atrial valve stent, wherein the expandable atrial valve stent is inserted into the expandable ventricular valve stent and the expandable atrial valve stent and the expandable ventricular valve stent are connected to each other such that the one or more atrial clamp jaws and the one or more ventricular clamp jaws are resiliently biased towards each to form a spring like clamp. In some embodiments, the expandable atrial valve stent and the expandable ventricular valve stent are connected together by suturing them together, whereas in some other embodiments the two stents are connected together by welding them together.

In some embodiments of the prosthetic mitral valve set forth above, one or both of the expandable atrial valve stent and the expandable ventricular valve stent are comprised of a shape memory material. In some such embodiments, the expandable ventricular valve stent has a deployed configuration and a shape set configuration such that at least a portion of the ventricular clamp jaws is more distal to the outflow end in the shape set configuration than in the deployed configuration.

Some embodiments of the instant technology are directed to a method of implanting a prosthetic mitral valve at a native mitral valve of a patient's heart having a left atrium and a left ventricle, the native mitral valve having a native mitral valve annulus and native mitral valve leaflets, wherein the native mitral valve annulus has an atrium side that faces the left atrium of the heart and a ventricle side that faces the left ventricle of the heart, and wherein the prosthetic mitral valve comprises an valve stent having an inflow end and an outflow end, one or more atrial clamp jaws projecting radially outwards and connected to the valve stent, one or more ventricular clamp jaws projecting radially outwards and also connected to the valve stent, and a plurality of prosthetic leaflets coupled to the valve stent at commissure attachment features of the valve stent, wherein when the prosthetic mitral valve is deployed at the site of the native mitral valve, the ventricular clamp jaws are deployed on the ventricle side of the native mitral valve annulus and atrial clamp jaws are deployed on the atrial side of the native mitral valve annulus such that the atrial clamp jaws and ventricular clamp jaws are sufficiently resiliently biased with respect to each other to grip the native mitral valve leaflets and the native mitral valve annulus between them, the method comprising crimping the prosthetic mitral valve under a sheath of a delivery catheter such that the one or more atrial clamp jaws and the one or more ventricular clamp jaws are crimped under a proximal part of the sheath of the delivery catheter and the outflow end of the valve stent is crimped in a distal part of the sheath of the delivery catheter, introducing the delivery catheter into the patient's body through percutaneous access moving the delivery catheter through the patient's body until the distal end of the delivery catheter is inside the left atrium of the patient's heart, pulling the sheath of the delivery catheter proximally to release the ventricular clamp jaws, advancing the delivery catheter through the left atrium and into the left ventricle of the patient's heart until the one or more ventricular clamp jaws have been pushed distally far enough into the left ventricle to be clear of the distal edges of native mitral valve leaflets pulling the delivery catheter in a proximal direction until the one or more ventricular clamp jaws abuts the ventricular side of the native mitral valve annulus pushing the native mitral valve leaflets against the native mitral valve annulus, pushing the distal part of the sheath of the delivery catheter to release the outflow end of the valve stent, withdrawing the proximal part of the sheath of the delivery catheter to release the one or more atrial clamp jaws such that the one or more atrial clamp jaws lie completely on the atrium side of the native mitral valve annulus touching at least some portion of the atrium side of the native mitral valve annulus, and

removing the delivery catheter from the patient's body.

In some embodiments of the method set forth above, the prosthetic mitral valve further comprises a circumferential suture connected to the one or more atrial clamp jaws and one or more removeable suture loops looped across the circumferential suture and threaded through the delivery catheter such that the free ends of the removeable suture loops exit through the proximal handle of the delivery catheter, the method further comprises tugging on the one or more removeable suture loops to adjust the placement of the prosthetic mitral valve. Further in some embodiments of the method, when the atrial clamp jaws are released on the atrial side of the native mitral valve annulus, the native mitral valve leaflets are confined to a region that is bounded on one side by the ventricular side of the native mitral valve annulus and on the other side by a plane the minimal longitudinal distance of which from the ventricular side of the native mitral valve annulus is less than 10 mm, and in some embodiments said minimum longitudinal distance is less than 6 mm, and in yet other embodiments said minimum longitudinal distance is less than 4 mm.

In some other embodiments of the method set forth above after the atrial clamp jaws are released on the atrial side of the native mitral valve annulus and the placement of the valve has been adjusted, the native mitral valve leaflets are confined to a region that is bounded on one side by the ventricular side of the native mitral valve annulus and on the other side by a plane the minimal longitudinal distance of which from the ventricular side of the native mitral valve annulus is less than 10 mm. In some other embodiments said minimum longitudinal distance is less than 6 mm, and in yet other embodiments, said minimum longitudinal distance is less than 4 mm.

Some embodiments of the instant technology are directed to prosthetic heart valve system for implantation at a native mitral valve wherein the mitral valve has an annulus and leaflets. In one embodiment, the prosthetic mitral valve system for implantation at a native mitral valve of a heart having a left atrium and a left ventricle, the native mitral valve having a native mitral valve annulus and native mitral valve leaflets, wherein the native mitral valve annulus has an atrium side that faces the left atrium of the heart and a ventricle side that faces the left ventricle of the heart, comprises a valve dock comprising a dock stent having an inflow end and an outflow end, one or more inflow clamp jaws projecting radially outwards and connected to the inflow end of the dock stent, and one or more ventricular clamp jaws projecting radially outwards and also connected to the inflow end of the dock stent, and a prosthetic mitral valve that can be docked inside the valve dock, wherein in the shape set configuration of the valve dock, at least some portion of the ventricular clamp jaws is further upstream of some portion of the inflow clamp jaws. In such a prosthetic mitral valve system the valve dock can also comprise one or more sacrificial prosthetic leaflets. In some embodiments, such a prosthetic mitral valve system has one or more ventricular clamp jaws that are atraumatic.

Further, in one embodiment, the one or more ventricular clamp jaws can comprise two sides, where one end of each of the sides is connected to form the valley end of the one or more ventricular clamp jaws and the other end of each of the sides is connected to the inflow end of the dock stent, wherein in the shape set configuration of the valve dock, the valley end of the one or more ventricular clamp jaws is bent in the direction of the outflow end of the dock stent.

In another embodiment, the prosthetic mitral valve system is such that when the valve dock is deployed at or adjacent to the native mitral valve annulus, the one or more ventricular clamp jaws are on the ventricle side of the native mitral valve annulus and the one or more of the inflow clamp jaws are on the atrium side of the native mitral valve annulus. In such an embodiment, upon deployment of the valve dock the native mitral valve leaflets are pushed up towards the native mitral valve annulus and the native mitral valve leaflets and the native mitral valve annulus are clamped between the one or more inflow clamp jaws and the one or more ventricular clamp jaws. In another embodiment, the clamping of the native mitral valve leaflets presses the native mitral valve leaflets into the native mitral valve annulus such that the native mitral valve leaflets are substantially confined to a region that does not extend more than 4 millimeters downstream of the most downstream point of the inflow clamp jaws.

In some embodiments, the prosthetic mitral valve further comprises an valve stent, the valve stent having a valve inflow end and a valve outflow end, a plurality of prosthetic leaflets coupled to the valve stent at commissure attachment features of the valve stent, and one or more atrial clamp jaws extending radially outwards from the periphery of the valve stent and attached to valve inflow end, wherein the prosthetic mitral valve can be implanted by docking it inside the valve dock such that upon expansion of the valve stent the valve stent will form an interference fit with the inside surface of the dock stent, and wherein upon implantation of the prosthetic mitral valve at or adjacent to the native mitral valve annulus the one or more atrial clamp jaws of the dock will be positioned on the atrium side of the native mitral valve annulus.

Another embodiment is a prosthetic mitral valve for implantation at a native mitral valve of a heart having a left atrium and a left ventricle, the native mitral valve having a native mitral valve annulus and native mitral valve leaflets, wherein the native mitral valve annulus has an atrium side that faces the left atrium of the heart and a ventricle side that faces the left ventricle of the heart, wherein the prosthetic mitral valve comprises a valve stent having an inflow end and an outflow end, one or more atrial clamp jaws projecting radially outwards and connected to the inflow end of the valve stent, and one or more ventricular clamp jaws projecting radially outwards and also connected to the inflow end of the valve stent, and one or more prosthetic mitral valve leaflets attached to the inside surface of the valve stent, wherein in the shape set configuration of the prosthetic mitral valve, at least some portion of the ventricular clamp jaws is further upstream of some portion of the atrial clamp jaws. In some embodiments, the one or more ventricular clamp jaws are atraumatic. In one embodiment of such a prosthetic mitral valve system the one or more ventricular clamp jaws comprise two sides, wherein at one end of each side the two sides are connected to each other to form a valley end of the one or more ventricular clamp jaws and the other end of each side the two sides are connected to the inflow end of the valve stent, wherein in the shape set configuration of the prosthetic mitral valve, the valley end of the one or more ventricular clamp jaws is bent in the direction of the outflow end of the valve stent. In one embodiment of this prosthetic mitral valve system when the prosthetic mitral valve is deployed at or adjacent to the native mitral valve annulus, the one or more ventricular clamp jaws are on the ventricle side of the native mitral valve annulus and the one or more of the atrial clamp jaws are on the atrium side of the native mitral valve annulus. In this case, upon deployment of the prosthetic mitral valve the native mitral valve leaflets are pushed up towards the native mitral valve annulus and the native mitral valve leaflets and the native mitral valve annulus are clamped between the one or more atrial clamp jaws and the one or more ventricular clamp jaws. In some embodiments, the clamping of the native mitral valve leaflets presses the native mitral valve leaflets into the native mitral valve annulus such that the native mitral valve leaflets are substantially confined to a region that does not extend more than 4 millimeters downstream of the most downstream point of the atrial clamp jaws.

The disclosure further provides systems for delivery of prosthetic mitral valve assemblies and other devices using endovascular or other minimally invasive forms of access. For example, embodiments of the present technology provide a system to treat a native mitral valve of a patient, wherein the system comprises a prosthetic mitral valve device to treat the native mitral valve as described herein and a catheter having a lumen configured to retain said device within the catheter. Such a system can include an elongated catheter body having a distal end and a proximal end, and a sheath housing coupled to the distal end of the catheter body and having a closed end and an open end. The system can further include a prosthetic valve device having a collapsed configuration and an expanded configuration. The prosthetic valve device can be positionable in the housing in the collapsed configuration and can be releasable proximally from the housing by moving an actuator.

In yet another aspect, embodiments of the present technology provide methods of treating a heart valve of a patient. An embodiment is a method of implanting a prosthetic mitral valve system at a native mitral valve of a patient's heart having a left atrium and a left ventricle, the native mitral valve having a native mitral valve annulus and native mitral valve leaflets, wherein the native mitral valve annulus has an atrium side that faces the left atrium of the heart and a ventricle side that faces the left ventricle of the heart, and wherein the prosthetic mitral valve system comprises a valve dock comprising a dock stent having an inflow end and an outflow end, one or more inflow clamp jaws projecting radially outwards and connected to the inflow end of the dock stent, and one or more ventricular clamp jaws projecting radially outwards and also connected to the inflow end of the dock stent, and a prosthetic mitral valve that can be docked inside the valve dock, wherein in the shape set configuration of the valve dock, at least some portion of the ventricular clamp jaws is further upstream of some portion of the inflow clamp jaws, wherein the method comprises crimping the valve dock under a sheath of a first delivery catheter such that the one or more inflow clamp jaws and the one or more ventricular clamp jaws are aligned proximally parallel to the longitudinal axis of the first delivery catheter such that the angle between the one or more inflow clamp jaws and the dock stent is about 180 degrees, introducing the first delivery catheter into the patient's body through percutaneous access and moving it through the patient's body until the distal end of the first delivery catheter is inside the left ventricle of the patient's heart, withdrawing the sheath of the first delivery catheter to release the valve dock such that the inflow end of the dock stent is distal to the free edge of at least one of the native mitral valve leaflets, withdrawing the sheath of the first delivery catheter to release the one or more ventricular clamp jaws such that the one or more ventricular clamp jaws lie completely on the ventricle side of the native mitral valve annulus, pulling the first delivery catheter in a proximal direction until the inflow end of the dock stent is at or adjacent to the atrium side of the native mitral valve annulus, withdrawing the sheath of the first delivery catheter to release the one or more inflow clamp jaws such that the one or more inflow clamp jaws lie completely on the atrium side of the native mitral valve annulus touching at least some portion of the atrium side of the native mitral valve annulus, and removing the first delivery catheter from the patient's body. In one embodiment of this method, after the one or more ventricular clamp jaws and the one or more inflow clamp jaws have been released, the minimum longitudinal distance between the ventricular clamp jaws and the inflow clamp jaws is less than 4 millimeters. The prosthetic mitral valve system can further comprises a prosthetic mitral valve comprising an valve stent, the valve stent having a valve inflow end and a valve outflow end, a plurality of prosthetic leaflets coupled to the valve stent at commissure attachment features of the valve stent, and one or more atrial clamp jaws extending radially outwards from the periphery of the valve stent and attached to valve inflow end, wherein the prosthetic mitral valve can be implanted by docking it inside the valve dock such that upon expansion of the valve stent the valve stent will form an interference fit with the inside surface of the dock stent, and the method of implanting the prosthetic mitral valve system can further comprise crimping the prosthetic mitral valve under a sheath of a second delivery catheter such that the one or more atrial clamp jaws are aligned proximally parallel to the longitudinal axis of the second delivery catheter such that the angle between the one or more atrial clamp jaws and the valve stent is about 180 degrees, introducing the second delivery catheter into the patient's body through percutaneous access and moving it through the patient's body until the distal end of the second delivery catheter is past the outflow end of the dock stent inside the left ventricle of the patient's heart. withdrawing the sheath of the second delivery catheter to release the valve stent such that the valve stent is anchored inside the dock stent, pulling the second delivery catheter in a proximal direction until the inflow end of the valve stent is at or adjacent to the atrium side of the native mitral valve annulus, withdrawing the sheath of the second delivery catheter to release the one or more atrial clamp jaws such that the one or more atrial clamp jaws lie completely on the atrium side of the native mitral valve annulus touching at least some portion of the atrium side of the native mitral valve annulus, and removing the second delivery catheter from the patient's body.

Another embodiment is a method of implanting a prosthetic mitral valve at a native mitral valve of a patient's heart having a left atrium and a left ventricle, the native mitral valve having a native mitral valve annulus and native mitral valve leaflets, wherein the native mitral valve annulus has an atrium side that faces the left atrium of the heart and a ventricle side that faces the left ventricle of the heart, and wherein the prosthetic mitral valve comprises a prosthetic mitral valve comprising a valve stent having a valve inflow end and a valve outflow end, one or more atrial clamp jaws projecting radially outwards and connected to the valve inflow end, and one or more ventricular clamp jaws projecting radially outwards and also connected to the valve inflow end, wherein in the shape set configuration of the prosthetic mitral valve, at least some portion of the ventricular clamp jaws is further upstream of some portion of the atrial clamp jaws, wherein the method comprises crimping the prosthetic mitral valve under a sheath of a first delivery catheter such that the one or more atrial clamp jaws and the one or more ventricular clamp jaws are aligned proximally parallel to the longitudinal axis of the first delivery catheter such that the angle between the one or more atrial clamp jaws and the valve stent is about 180 degrees, introducing the first delivery catheter into the patient's body through percutaneous access and moving it through the patient's body until the distal end of the first delivery catheter is inside the left ventricle of the patient's heart, withdrawing the sheath of the first delivery catheter to release the valve stent such that the valve inflow end of the valve stent is distal to the free edge of at least one of the native mitral valve leaflets, withdrawing the sheath of the first delivery catheter to release the one or more ventricular clamp jaws such that the one or more ventricular clamp jaws lie completely on the ventricle side of the native mitral valve annulus, pulling the first delivery catheter in a proximal direction until the inflow end of the valve stent is at or adjacent to the atrium side of the native mitral valve annulus, withdrawing the sheath of the first delivery catheter to release the one or more atrial clamp jaws such that the one or more atrial clamp jaws lie completely on the atrium side of the native mitral valve annulus touching at least some portion of the atrium side of the native mitral valve annulus, and removing the first delivery catheter from the patient's body. In one embodiment of this method, after the one or more ventricular clamp jaws and the one or more atrial clamp jaws have been released, the minimum longitudinal distance between the ventricular clamp jaws and the atrial clamp jaws is less than 4 millimeters.

Another embodiment is a method of treating disease of a native mitral valve having a native annulus and native leaflets using a prosthetic mitral valve system, wherein the prosthetic mitral valve system comprises a valve dock comprising a dock stent having an inflow end and an outflow end, one or more inflow clamp jaws projecting radially outwards and connected to the inflow end of the dock stent, and one or more ventricular clamp jaws projecting radially outwards and also connected to the inflow end of the dock stent, and a prosthetic mitral valve that can be docked inside the valve dock, wherein in the shape set configuration of the valve dock, at least some portion of the ventricular clamp jaws is further upstream of some portion of the inflow clamp jaws, the method comprises placing the valve dock in a collapsed state at or adjacent to the native mitral valve annulus, deploying the valve dock such that inflow clamp jaws of the valve dock are deployed on the atrium side of the native annulus and the ventricular clamp jaws of the valve dock are deployed on the ventricle side of the native annulus thereby sandwiching the native mitral valve leaflets and the native mitral valve annulus between the inflow clamp jaws and the ventricular clamp jaws of the valve dock, and deploying the prosthetic mitral valve inside the valve dock.

The devices and methods disclosed herein can be configured for treating non-circular, asymmetrically shaped valves and bileaflet or bicuspid valves, such as the mitral valve. It can also be configured for treating other atrio-ventricular valves of the heart such as the tricuspid valve. Many of the devices and methods disclosed herein can further provide for long-term (e.g., permanent) and reliable anchoring of the prosthetic device even in conditions where the native heart valve may experience gradual enlargement or distortion.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components can be shown as transparent in certain views for clarity of illustration only and not to indicate that the illustrated component is necessarily transparent.

FIG. 1A is a perspective view of an embodiment of a valve dock in its shape set configuration.

FIG. 1B is a perspective view of an embodiment of a valve dock in its shape set configuration that also shows sacrificial prosthetic leaflets.

FIG. 1C is another perspective view of an embodiment of a valve dock in its shape set configuration that also shows sacrificial prosthetic leaflets.

FIG. 2 is a perspective view of a detail of an embodiment of a valve dock in its shape set configuration showing the relative location of inflow clamp jaws and ventricular clamp jaws with respect to the inflow plane of the valve dock.

FIG. 3A perspective view of an embodiment of a valve dock in its shape set configuration that shows barbs on ventricular clamp jaws.

FIG. 3B perspective view of an embodiment of a valve dock in its shape set configuration that shows fabric covers on ventricular clamp jaws.

FIG. 4A is a perspective view of a detail of an embodiment of a valve dock in its shape set configuration showing the strain relief elements.

FIG. 4B is a 2-dimensional as cut view of an embodiment of a valve dock showing how ventricular clamp jaws and inflow clamp jaws will be aligned with respect to the dock stent when the valve dock is crimped under a sheath for delivery inside a patient's heart.

FIGS. 5A and 5B are perspective views of an embodiment of a prosthetic mitral valve in its shape set configuration. FIG. 5A illustrates the length, H, of the dock stent excluding eyelets. FIG. 5B also illustrates the height, HL, of the prosthetic mitral valve leaflets.

FIG. 5C is a plan view of an embodiment of a prosthetic mitral valve in its shape set configuration.

FIG. 6 is a 2-dimensional as cut view of an embodiment of a prosthetic mitral valve showing how atrial clamp jaws will be aligned with respect to the valve stent when the prosthetic mitral valve is crimped under a sheath for delivery inside a patient's heart, i.e., the angle between the atrial clamp jaws and the valve stent will be about 180 degrees.

FIGS. 7A-7B are perspective views of an embodiment of a prosthetic mitral valve system in its shape set configuration.

FIG. 8A shows an embodiment of a delivery catheter delivering a valve dock inside a patient's heart.

FIG. 8B shows an embodiment of a valve dock deployed at or adjacent to the native mitral valve annulus of a patient being treated for mitral valve disease.

FIG. 8C shows an embodiment of a valve dock deployed at or adjacent to the native mitral valve annulus of a patient being treated for mitral valve disease.

FIG. 8D is a schematic illustration of an embodiment of a valve dock deployed at or adjacent to the native mitral valve of a patient being treated for mitral valve disease showing the native mitral valve leaflets clamped between ventricular clamp jaws and inflow clamp jaws of the valve dock.

FIG. 9A shows an embodiment of a delivery catheter delivering a prosthetic mitral valve inside a patient's heart.

FIG. 9B shows an embodiment of a prosthetic mitral valve docked inside a valve dock deployed at or adjacent to the native mitral valve annulus of a patient being treated for mitral valve disease.

FIG. 9C shows an embodiment of a prosthetic mitral valve docked inside a valve dock deployed at or adjacent to the native mitral valve annulus of a patient being treated for mitral valve disease showing the native mitral valve leaflets confined to a region with width W.

FIG. 9D is a schematic illustration of an embodiment of a prosthetic mitral valve docked inside a valve dock deployed at or adjacent to the native mitral valve of a patient being treated for mitral valve disease showing the native mitral valve leaflets clamped between ventricular clamp jaws and inflow clamp jaws of the valve dock and the atrial clamp jaws of the prosthetic mitral valve.

FIG. 10A-10B are perspective views of an embodiment of a valve dock in its shape set configuration.

FIGS. 11A-11B are perspective views of an embodiment of a prosthetic mitral valve system in its shape set configuration.

FIG. 12A shows an embodiment of a delivery catheter delivering a valve dock inside a patient's heart.

FIG. 12B shows an embodiment of a valve dock deployed at or adjacent to the native mitral valve annulus of a patient being treated for mitral valve disease.

FIG. 13 shows an embodiment of a valve dock deployed at or adjacent to the native mitral valve annulus of a patient being treated for mitral valve disease.

FIG. 14 shows an embodiment of a delivery catheter delivering a prosthetic mitral valve inside a patient's heart.

FIG. 15 shows an embodiment of a prosthetic mitral valve docked inside a valve dock deployed at or adjacent to the native mitral valve annulus of a patient being treated for mitral valve disease.

FIG. 16 shows an embodiment of a prosthetic mitral valve docked inside a valve dock deployed at or adjacent to the native mitral valve annulus of a patient being treated for mitral valve disease showing that the native mitral valve leaflets are confined in a narrow region of width W.

FIG. 17 shows an embodiment of a single catheter delivery system for delivery of an embodiment of a valve dock and a prosthetic mitral valve mounted on the same delivery catheter.

FIG. 18 shows an embodiment of a valve dock deployed at or adjacent to the native mitral valve annulus of a patient where anchor legs of valve dock are held inside the nose cone of the delivery catheter and the prosthetic mitral valve is held crimped under the sheath of the delivery catheter.

FIG. 19 shows an embodiment of a delivery catheter positioning an embodiment of a prosthetic mitral valve for docking the valve inside the deployed valve dock of FIG. 18.

FIG. 20 shows an embodiment of a prosthetic mitral valve docked inside the deployed valve dock of FIG. 18.

FIGS. 21A-21B are perspective views of an embodiment of a prosthetic mitral valve in its undeployed state.

FIG. 22A-22B shows perspective views of the embodiment of the prosthetic mitral valve of FIGS. 21A and 21B in its shape set configuration.

FIG. 23 shows an embodiment of a prosthetic mitral valve where a circumferential suture is connected to the atrial clamp jaws and removeable suture loops are looped through the circumferential suture loop.

FIG. 24-FIG. 28 are schematic figures showing various stages of the delivery at or adjacent to the native mitral valve annulus of a prosthetic mitral valve crimped under a delivery catheter sheath (figures are not to scale).

FIG. 29 shows an embodiment of a prosthetic mitral valve deployed at or adjacent to the native mitral valve annulus of a patient being treated for mitral valve disease showing that the native mitral valve leaflets and native mitral valve annulus are clamped between atrial clamp jaws and ventricular clamp jaws.

### DETAILED DESCRIPTION OF THE INVENTION

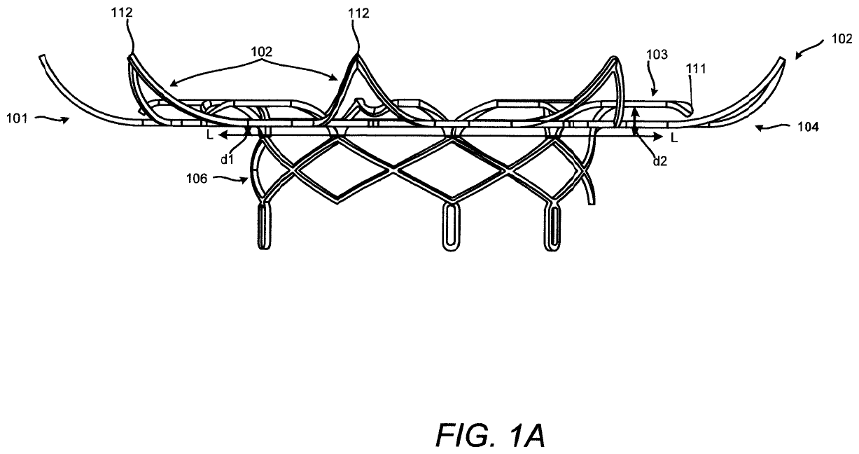

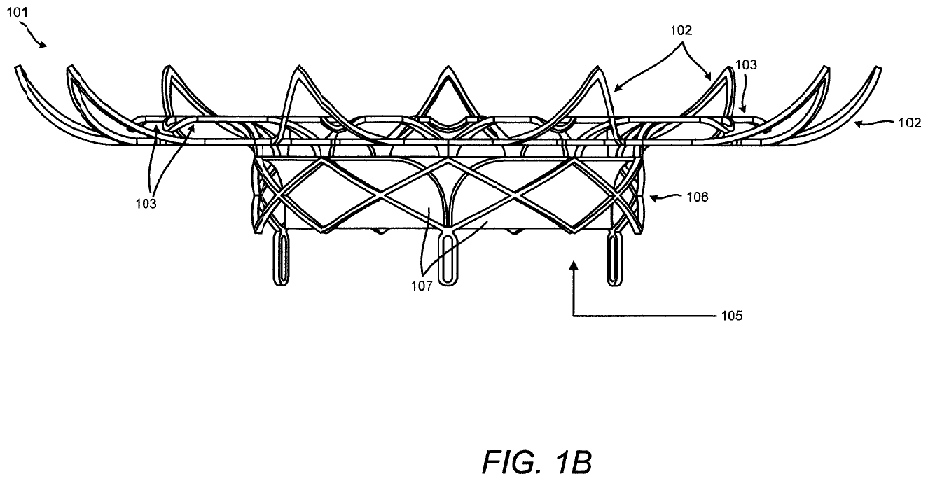

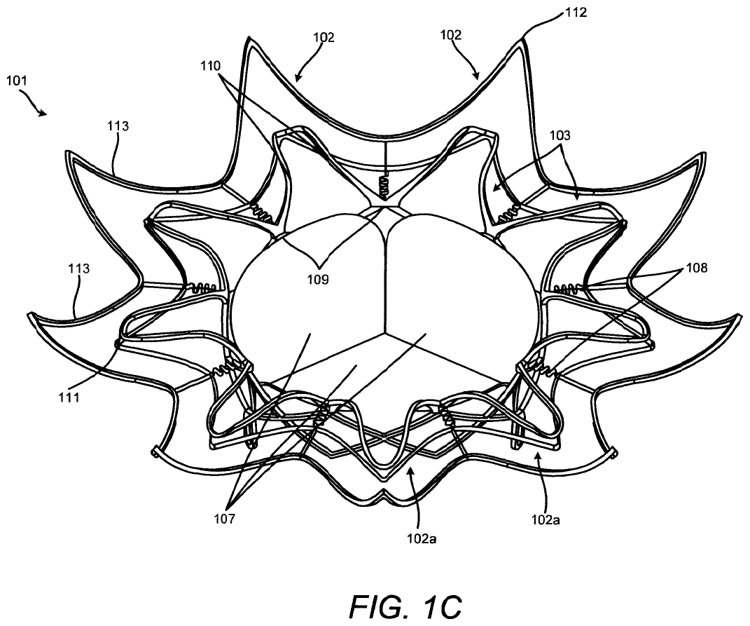

Specific details of several embodiments of prosthetic heart valves and systems and methods of implanting them are described below with reference to FIGS. 1-29. Although many of the embodiments are described below with respect to devices, systems, and methods for percutaneous replacement of a native mitral valve using prosthetic valve devices, other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below.

With regard to the terms “distal” and “proximal” within this description, unless otherwise specified, the terms can reference a relative position of the portions of a prosthetic valve device and/or an associated delivery device with reference to an operator and/or a location in the vasculature or heart. For example, in referring to a delivery catheter suitable to deliver and position various prosthetic valve devices described herein, “proximal” can refer to a position closer to the operator of the device or an incision into the vasculature, and “distal” can refer to a position that is more distant from the operator of the device or further from the incision along the vasculature (e.g., the end of the catheter). With respect to a prosthetic heart valve device, the terms “proximal” and “distal” can refer to the location of portions of the device with respect to the direction of blood flow. For example, proximal can refer to an upstream position or a position of blood inflow, and distal can refer to a downstream position or a position of blood outflow. Thus, for example, in the context of left side of the heart, proximal refers to atrial or upstream position or position of blood inflow; and distal refers to ventricular or downstream position or position of blood outflow.

The term “clamp jaws” as used throughout this application is defined to mean an element comprising at least two side elements wherein one end of each side element is in some embodiments connected to the other by a bridge element but in other embodiments these two ends are not connected, and the other end of each side element is connected to a main body, for example, a stent body, for example the valve dock stent or the prosthetic mitral valve stent herein. As discussed below, the ventricular clamp jaws illustrated in the embodiments shown here are “U” shaped, where the legs of the “U” form the side elements, the valley of the “U” is the bridge element which connects one end of each leg to that of the other, and at the other end each of the legs is connected to the dock stent or valve stent as the case maybe. In the illustrated embodiments, the inflow clamp jaws and atrial clamp jaws are “V” shaped, where the legs of the “V” form the side elements, the valley of the “V” is the bridge element where one end of each leg is connected to that of the other, and at the other end each of the legs is connected to the dock stent or valve stent as the case maybe. However, other shapes are contemplated. For example, ventricular clamp jaws as well as inflow clamp jaws and atrial clamp jaws can be in the shape of a “V” and all types of jaws can be in the shape of a “U” and one can be one shape and the other can be the other shape. In general, under the broad definition of clamp jaws hereinabove, various shapes are contemplated, such as “W” shaped clamp jaws, “U” or “V” or “W” shaped clamp jaws, or other shapes of clamp jaws, or clamp jaws that have one or more nested “U” or “V” or “W” or other shapes of clamp jaws inside them. Thus, for example, in the embodiment shown in FIG. 1C, the inflow clamp jaws 102 have nested in them clamp jaws 102a.

The term “shape set configuration” of a device herein is defined as the state of a device comprising a shape memory material wherein the device assumes the shape that it was set to during the shape setting process, which is a process of giving a desired shape to anything, such as devices in the form of mesh tubes, comprising a shape memory material, e.g., nitinol, such that when the device is maintained above a certain temperature and is not otherwise constrained, it will be in the desired shape. When a device has been implanted inside the body of a patient, or deployed, the device will tend towards its shape set or shape set configuration but may not reach that exact state because of interference with structural elements of a patient's anatomy, e.g., native mitral valve leaflets and the native mitral valve annulus, or with structural elements of the device itself. This will cause components that are not able to return to their shape set configuration to be resiliently biased.

A third configuration for devices herein is the delivery configuration where the device is not in a shape set configuration—the device is crimped underneath the sheath of a catheter for delivery of the device inside the body of a patient.

For ease of reference, throughout this disclosure identical reference numbers and/or letters are used to identify similar or analogous components or features, but the use of the same reference number does not imply that the parts should be construed to be identical. Indeed, in many examples described herein, the identically numbered parts are distinct in structure and/or function. The headings provided herein are for convenience only.

### Cardiac and Mitral Valve Physiology

The human heart lies slightly to the left of center in the chest. It is shaped roughly like an inverted triangle with the pointed end, the apex, directed downward and to the left. The heart is composed almost entirely of a muscle called the myocardium. The exterior surface of the myocardium is called the epicardium and the interior surface is called the endocardium. The heart is it two-sided pump. The right side of the heart serves as the pump for the pulmonary circuit; it receives unoxygenated blood from the systems veins and pumps it to the lungs. The left side, the pump for the systemic circuit, receives oxygenated blood from the pulmonary veins and pumps it to the tissues of the body.

On each side of the heart there are two chambers. The atria are inflow chambers which receive blood from the veins and deliver it to the ventricles. The ventricles are pumping chambers and squeeze blood out through the arteries by contracting periodically. The wall between the atria is call the atrial or inter-atrial septum. Thus, the heart consists of four chambers. The chambers on the right side of the heart, the right atrium (RA) and right ventricle (RV) are in the pulmonary circuit. The chambers on the left side of the heart, the left atrium (LA) and left ventricle (LV), are in the systemic circuit.

To Prevent the backflow of blood as it passes through the heart, it contains four valves, two on each side. These valves comprise leaflets (LF) which regulate blood flow by allowing blood to flow in only one direction. Between each atrium and ventricle there is an atrio-ventricular (AV) valve to prevent backflow when the ventricles contract. The one on the right side is called the tricuspid valve (TV) and the one on the left side is called the mitral valve (MV). Certain embodiments of this invention relate to replacement of a malfunctioning or deceased AV valve, i.e., a valve that is allowing blood to backflow, a phenomenon known as regurgitation, as opposed to a healthy functioning heart valve which only allows blood flow in one direction and does not allow blood to backflow in the opposite direction.

When the ventricles contract, high pressure is developed in them. To prevent the leaflets of the AV valves, TV and MV, from ballooning back into the Atria during ventricular contraction, the leaflets are supported by other structures in the heart. The ends of the AV valve leaflets are attached to strong, tendon like strings called chordae tendinae (CT). The chordae tendinae are, in turn, attached to finger-like papillary muscles (PM) in the ventricular myocardium. These muscles exert tension on the leaflets of the TV and MV during ventricular contraction and prevent them from prolapsing back into the atria.

The MV comprises a pair of leaflets having free edges FE which meet evenly, or “coapt” to close. The opposite ends of the leaflets are attached to the surrounding heart structure via an annular region of tissue referred to as the annulus AN. When the leaflets are in the coapted state, the surface of the leaflets that faces the atrium is referred to as the atrial surface and the surface of the leaflets that faces the ventricle is referred to as the ventricular surface. In the open state of the leaflets, the atrial surfaces of the leaflets face each other and their ventricular surfaces face the walls of the ventricles.

### Percutaneous Delivery of Prosthetic Mitral Valve System

Percutaneous heart valve replacement is a well-known interventional procedure involving the insertion of an artificial heart valve using a catheter, rather than through open heart surgery. An expandable prosthetic heart valve is crimped onto a catheter and deployed without removing the diseased native valve at the site of the diseased native valve. “Percutaneous” means that the catheter accesses the heart via a remote entry portal through the skin, typically using a surgical cut down procedure or a minimally invasive procedure. Such a procedure does not require heart-lung bypass. Potential advantages include decreased recovery time and lower surgical risk. The entry portal is typically either via the femoral vein or artery, or directly through the myocardium via the apical region of the heart. The prosthetic heart valve can also be delivered and implanted through the heart wall (the “transapical” approach), through the subclavian artery, through the axillary artery, and through the ascending aorta. Further, two types of delivery paths can potentially be used: antegrade or retrograde. In the antegrade path, the prosthetic mitral valve can approach the implantation site by crossing the inter-atrial septum into the left atrium. Alternatively, in the retrograde path the left ventricle is entered via the aortic valve.

Once percutaneous access is achieved, interventional tools and supporting catheter(s) may be advanced to the heart intravascularly and positioned adjacent the target cardiac valve in a variety of manners, as described herein.

One exemplary antegrade delivery method involves, after obtaining percutaneous access to the femoral vein, advancing a catheter having a needle or a guidewire into the right atrium RA through the inferior vena cava IVC. When the catheter is on the anterior side of the inter-atrial septum, the needle or guidewire is made to penetrate the inter-atrial septum. Following this, in the case where a needle is used instead of a guidewire, a guidewire is exchanged for the needle and the catheter is withdrawn. By placing a catheter over a guidewire access to the left atrium through the inter-atrial septum is maintained. The catheter can then be used to deliver a prosthetic mitral valve and associated devices at or adjacent to the native mitral valve annulus.

Antegrade or trans-septal delivery has several advantages, including, without limitation, reduced risk to native mitral valve structures such as chordae tendinae, potentially more accurate centering and stabilization of the prosthetic mitral valve system, and reduction of risks associated with the retrograde approach which may involve going across the aortic valve.

An exemplary retrograde approach may involve accessing the mitral valve MV using an approach from the aortic arch AA, across the aortic valve AV, and into the left ventricle LV below the mitral valve MV. The aortic arch AA may be accessed through a conventional femoral artery access route, as well as through more direct approaches via the brachial artery, axillary artery, radial artery, or carotid artery. Such access may be achieved with the use of a guidewire. Once in place, a catheter may be tracked over the guidewire. Alternatively, a surgical approach may be taken through an incision in the chest, preferably intercostally without removing ribs, and placing a catheter through a puncture in the aorta itself. The catheter affords subsequent access to permit placement of a prosthetic valve device, as described in more detail herein.

The retrograde approach has certain advantages, for example, use of the retrograde approach can eliminate the need for a trans-septal puncture. The retrograde approach is also more commonly used by cardiologists and thus has the advantage of familiarity.

An additional approach to the mitral valve is via trans-apical puncture. In this approach, access to the heart is gained via thoracic incision, which can be a conventional open thoracotomy or sternotomy, or a smaller intercostal or sub-xyphoid incision or puncture. An access cannula is then placed through a puncture, sealed by a purse-string suture, in the wall of the left ventricle at or near the apex of the heart. The catheters and prosthetic devices of the invention may then be introduced into the left ventricle through this access cannula.

The trans-apical approach has the feature of providing a shorter, straighter, and more direct path to the mitral valve. Further, because it does not involve intravascular access, the trans-apical procedure can be performed by surgeons who may not have the necessary training in interventional cardiology to perform the catheterizations required in other percutaneous approaches.

Orientation and steering of the prosthetic valve device can be combined with many known catheters, tools and devices. Such orientation may be accomplished by gross steering of the device to the desired location and then refined steering of the device components to achieve a desired result.

Gross steering may be accomplished by a number of methods. A steerable guidewire may be used to introduce a catheter and the prosthetic treatment device into the proper position. The catheter may be introduced, for example, using a surgical cut down or Seldinger access to the femoral artery in the patient's groin. After placing a guidewire, the catheter may be introduced over the guidewire to the desired position. Alternatively, a shorter and differently shaped catheter could be introduced through the other routes described above.

A catheter may be pre-shaped to provide a desired orientation relative to the mitral valve. For access via the trans-septal approach, the catheter may have a curved, angled or other suitable shape at its tip to orient the distal end toward the mitral valve from the location of the septal puncture through which the catheter extends. For the retrograde approach, the catheter may have a pre-shaped J-tip configured to turn toward the native mitral valve annulus after it is advanced through the aortic valve. The catheter might also have pull-wires or other means to adjust its shape for more fine steering adjustment.

### Prosthetic Heart Valve System and Methods

Embodiments of the technology described herein can be used to treat one or more of the valves of the heart, and particular embodiments can be used for treatment of the mitral valve. Examples of a prosthetic heart valve system, its components and associated methods are described in this section with reference to FIGS. 1-29. As can be appreciated by a person of ordinary skill in the art, particular elements, substructures, advantages, uses, and/or other features of the embodiments described herein can be suitably interchanged, substituted or otherwise configured with one another.

Systems, devices and methods are provided herein for percutaneous implantation of prosthetic heart valves in a heart of a patient. In some embodiments, methods and devices are presented for the treatment of valve disease by percutaneous implantation of a prosthetic replacement heart valve. In one embodiment, the prosthetic replacement valve can be a prosthetic mitral valve device that can be implanted as a replacement of a native mitral valve between the left atrium and left ventricle in the heart of a patient. Another embodiment contemplates a prosthetic valve device that can be implanted as a replacement of any AV valve (e.g., a tricuspid valve) in the heart of the patient.

In one embodiment, the prosthetic heart valve system comprises a valve dock and a prosthetic mitral valve, wherein the valve dock is implanted at or adjacent to the annulus of the native mitral valve of the patient and the prosthetic mitral valve is implanted inside the valve dock to create the prosthetic mitral valve system described herein. FIGS. 1A-1C show isometric views of a valve dock 101 in a shape set configuration in accordance with an embodiment of the present technology. As shown in FIG. 1A, the valve dock includes a dock stent 106 which has an inflow end 104 which is the end that would be nearest to the left atrium of the patient's heart post implantation of the valve dock and an outflow end 105 which would be the end furthest from the left atrium of the patient's heart post implantation of the valve dock. Thus, blood flows from the left atrium into the inflow end and flows out of the outflow end into the left ventricle.

The dock stent 106 can be a tubular structure made of, for example, a wire mesh and can be radially collapsible and expandable between a radially expanded state and a radially compressed state for delivery and implantation at or adjacent to a native mitral valve annulus. The wire mesh can include metal wires or struts arranged in a lattice pattern. Dock stent 106 can be made of a shape-memory material, for example Nitinol, which makes the dock stent self-expandable from a radially compressed state to an expanded state. Alternatively, dock stent 106 can be plastically expandable from a radially compressed state to an expanded state using, for example, an inflatable balloon. Exemplary materials for such balloon expandable dock stents can be stainless steel, chromium alloys, and/or other materials known to persons of ordinary skill in the art.

In the interim period between implantation of valve dock 101 and implantation of the prosthetic mitral valve as discussed below, the native mitral valve leaflets cannot regulate blood flow between the left atrium and the left ventricle. To regulate blood flow during this interim period, in one embodiment, the valve dock 101 also includes sacrificial prosthetic leaflets 107 shown in FIGS. 1B and 1C, which are also perspective views of valve dock 101. Thus, valve dock 101 comprises a plurality of sacrificial prosthetic leaflets 107 supported by and/or within the dock stent 106. The plurality of sacrificial prosthetic leaflets 107 and concomitant structure serves to regulate blood flow through the valve dock prior to implantation of a prosthetic mitral valve. The sacrificial prosthetic leaflets 107 can comprise materials, such as bovine or porcine pericardial tissue or synthetic materials. The sacrificial prosthetic leaflets 107 can be mounted to the dock stent 106 using well-known techniques and mechanisms. For example, the sacrificial prosthetic leaflets 107 can be sutured to the dock stent 106 in a tricuspid arrangement, as shown in FIG. 1C.

As will be discussed below, when a prosthetic mitral valve is docked inside valve dock 101, sacrificial prosthetic leaflets 107 will be pushed aside by the valve stent of the prosthetic mitral valve and will cover the outside surface of the valve stent of the prosthetic mitral valve, thereby acting as a barrier to paravalvular leak (PVL).

As shown in FIG. 1A, connected at or adjacent to inflow end 104 of dock stent 106 are one or more inflow clamp jaws 102 and one or more ventricular clamp jaws 103. In the embodiment shown in FIG. 1C, there are nine inflow clamp jaws 102 and nine ventricular clamp jaws 103, arranged equidistant from each other around inflow end 104. Thus, the vertices of adjacent inflow clamp jaws 102 as well as adjacent ventricular clamp jaws 103 are 40 degrees apart from each other. In various embodiments, inflow clamp jaws 102 and/or ventricular clamp jaws 103 can be evenly spaced from each other, where adjacent clamp jaws are from 20-180 degrees apart. In other embodiments, inflow clamp jaws 102 and/or ventricular clamp jaws 103 can be unevenly spaced from each other. Thus, for example, one set of adjacent clamp jaws can be 20 degrees apart whereas another adjacent set can be 60 degrees apart.

In one embodiment of a valve dock, one or more ventricular clamp jaws 103 is in the shape of a “U” as shown in FIG. 1C, which is a perspective view of valve dock 101. In the embodiment shown in FIG. 1C, inflow clamp jaws 102 are in the shape of a “V.”

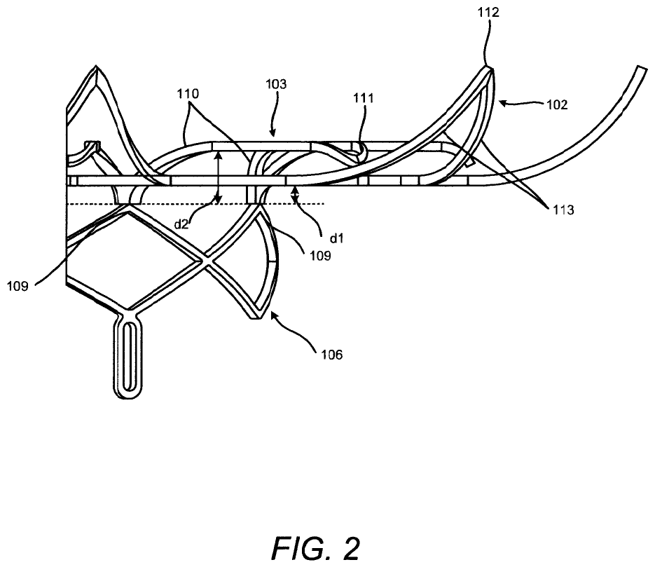

FIG. 2 is a partial perspective view of valve dock 101. As shown in FIG. 2, legs 110 of “U” shaped ventricular clamp jaw 103 are bent in the direction of dock stent 106 and are connected to it at 109. Ventricular clamp jaw 103 is also bent at its valley end 111 towards dock stent 106, such that end 111 will point away from the left atrium and also away from the inside wall of the left ventricle post implantation. The smoothly curved “U” shape of ventricular clamp jaw 103 with bent valley end 111 makes it atraumatic.

In the embodiment shown in FIG. 1C, inflow clamp jaws 102 are “V” shaped with legs 113 and end 112. As shown in FIG. 2, near end 112, legs 113 are curved up and away from the inflow end of dock stent 106, such that post implantation end 112 will point away from the left ventricle (or in the proximal direction) and also away from the inside wall of the left atrium making it atraumatic. The legs of the inflow clamp jaws are connected to the dock stent 106 at 109 via strain relieving element 108 as shown in FIG. 1C. In the embodiment shown in FIG. 1C, the inflow clamp jaws comprise clamp jaws 102a nested inside clamp jaws 102.

Inflow clamp jaws 102 and/or ventricular clamp jaws 103 may also be made atraumatic by wrapping the clamp jaws with tissue, such as bovine or porcine pericardium tissue, or by other materials such as polytetrafluoroethylene (PTFE).

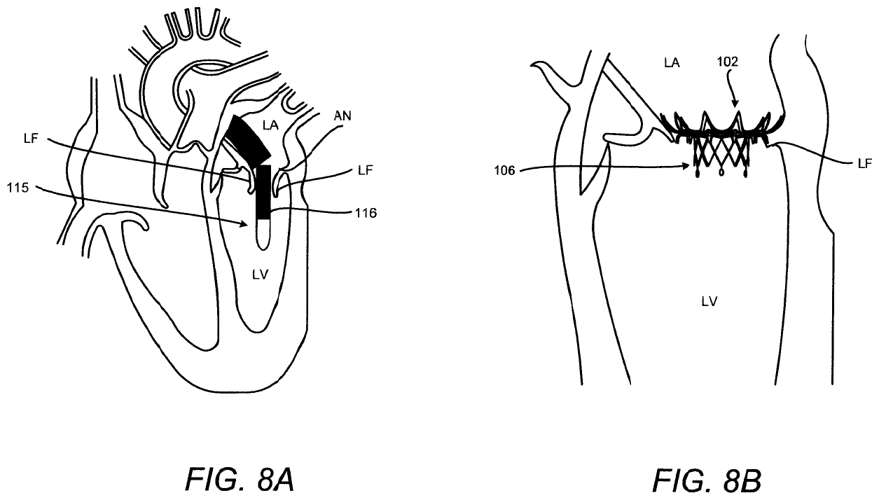

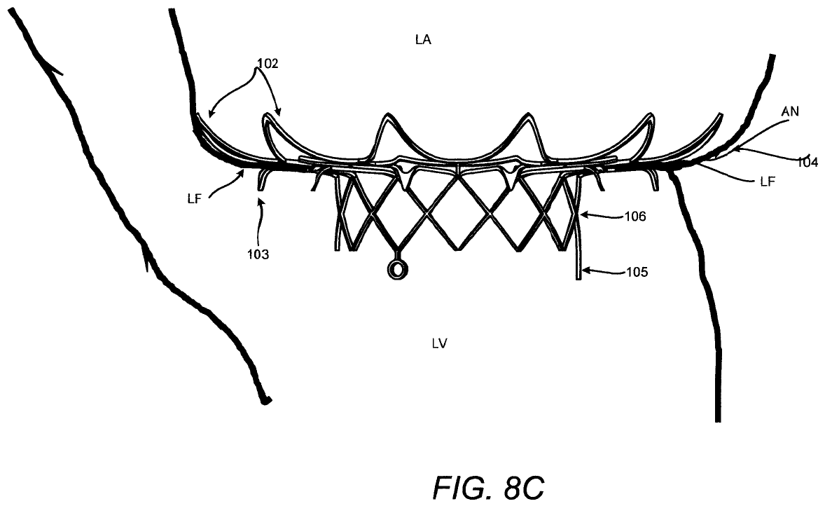

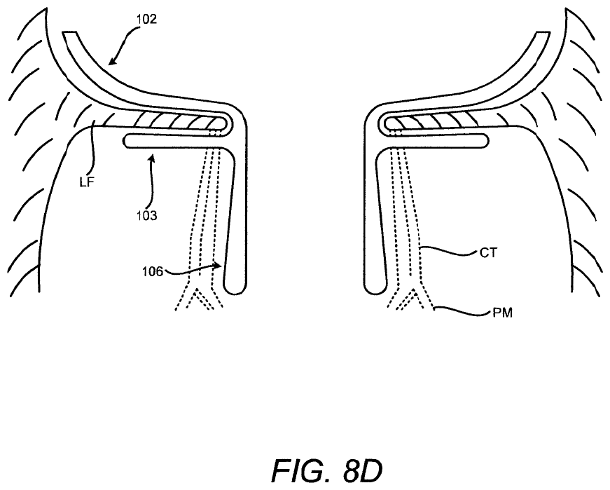

In the embodiment shown in FIGS. 1 and 2, the relative location of inflow clamp jaws 102 and ventricular clamp jaws 103 in the shape set configuration is such that when the valve dock is deployed or implanted at or adjacent to the native mitral valve annulus of a patient's heart, the two clamp jaws will act as a pair of forceps to clamp or pinch between them the native mitral valve leaflets and native mitral valve annulus as shown in FIGS. 8C and 8D and discussed hereinbelow. As shown in FIG. 1A, in the shape set of valve dock 101, a portion of the legs of ventricular clamp jaws 103 lies further upstream from line LL than a portion of the legs of inflow clamp jaws 102, where line LL is perpendicular to the longitudinal axis of dock stent 106 and lies in the inflow plane of the dock stent, i.e., the plane that contains inflow end 104. As will be discussed below, when the valve dock is deployed at or adjacent to the native mitral valve annulus, ventricular clamp jaws 103 will be positioned on the ventricular or downstream side of the native mitral valve annulus and the inflow clamp jaws will be positioned on the atrial or upstream side of the native mitral valve annulus, reversing their relative positions from the shape set configuration which means that the jaws have been elastically deformed from their shape set configuration, and, therefore, they will be resiliently biased to return to their shape state, thereby pinching or clamping the native mitral valve leaflets and native mitral annulus between them.

The relative location of inflow clamp jaws and the ventricular clamp jaws in the shape set configuration can be illustrated with reference to the embodiment shown in FIG. 1A. Longitudinal distance d from line LL to any point is defined as the distance measured from line LL to that point along a line that is perpendicular to line LL. If the point lies upstream of line LL (or proximally of line LL), d will be positive. For points that lie downstream of line LL (or distally of line LL), distance d will be negative.

As can be seen with reference to FIG. 1A and FIG. 2, the legs of the “U” shaped ventricular clamp jaws 103 have a section that is substantially parallel to line LL and furthest from line LL. Therefore, d2, the longitudinal distance between any point on this section and line LL, is the maximum longitudinal distance between any point on ventricular clamp jaws 103 and line LL. Similarly, the legs of the “V” shaped inflow clamp jaws 102 have a section that is substantially parallel to line LL and nearest to line LL. Therefore, d1, the longitudinal distance between any point on this section and line LL is the minimum longitudinal distance between any point on inflow clamp jaws 102 and line LL. Although, as illustrated, distances d1 and d2 are measured from line LL to sections of jaws 102 and 103, respectively, that are substantially parallel to line LL, in general, they are defined as: d1 is the minimum longitudinal distance between inflow clamp jaw 102 and line LL and d2 is the maximum longitudinal distance between ventricular clamp jaws 103 and line LL.

If we define maximum relative separation d3 as being equal to d2−d1, in the embodiment shown in FIG. 1A and FIG. 2, i.e., in the shape set configuration of the valve dock shown in these figures, d3>0. If d3 is >0, in the shape set configuration of valve dock 101, some portion of ventricular clamp jaws 103 lies further upstream (or further in the atrial direction) from line LL than some portion of inflow clamp jaws 102 as shown in FIGS. 1A and 2. Upon deployment the inflow clamp jaws will be further upstream (or further in the atrial direction) than the ventricular clamp jaws and the native mitral valve leaflets and the native mitral valve annulus will be in between the two jaws, which means that the clamp jaws are elastically deformed from their shape set configuration. This means that the ventricular clamp jaws and inflow clamp jaws are resiliently biased with respect to each other and will have a tendency to move relative to each other towards their shape set configuration. Because deployment of valve dock 101 is done in such a way that the native mitral valve leaflets and the native mitral valve annulus is trapped between them, the resilient forces between the two sets of clamp jaws will result in the native mitral valve leaflets and native mitral valve annulus being pinched or clamped between them.

In the embodiments shown in FIGS. 1A and 2, d3>0 and as discussed above this configuration of the ventricular and inflow clamp jaws in the shape set configuration of the valve dock will have the effect of these two sets of clamp jaws being resiliently biased with respect to each other when the valve dock is deployed, which will cause the native mitral valve leaflets and mitral valve annulus to be pinched or clamped between the ventricular and inflow clamp jaws. This effect will also obtain if d3=0. In that configuration, the ventricular clamp jaws and the inflow clamp jaws are equidistant. In other embodiments, d2 may be equal to d1 and in still other embodiments d2 may even be less than d1 as long as the inflow clamp jaws and ventricular clamp jaws are sufficiently close to each other so that upon deployment of valve dock 101, native mitral valve leaflets LF are pressed between the clamp jaws 102 and 103. In general, given the anatomy of the native mitral valve, if d3>−4 mm (i.e., d3 measured in millimeters is greater than minus 4), ventricular clamp jaws 103 and inflow clamp jaws 102 upon deployment, when ventricular clamp jaws are downstream of the native annulus and inflow clamp jaws are upstream of the native annulus, will be sufficiently resiliently biased to pinch or clamp the native mitral valve leaflets and native mitral valve annulus between them.

In the embodiment shown in FIGS. 1A and 2, sections of both the “U” shaped ventricular clamp jaws and the “V” shaped inflow clamp jaws are substantially parallel to line LL, are substantially parallel to each other and, therefore, the legs of the “U” shaped ventricular clamp jaws have a section that is substantially parallel to a section of the legs of the “V” shaped inflow clamp jaws. In other embodiments, inflow clamp jaws 102 and ventricular clamp jaws 103 can be such that they do not have any sections that are parallel to each other or to line LL. In such embodiments too, clamp jaws 102 and 103 would be resiliently biased so that the native mitral valve leaflets and the native mitral annulus is clamped between them if d3>−4 mm.

The radial lengths of the clamp jaws 102 and 103 should be sufficient to provide a stable and secure anchor for implanting the prosthetic mitral valve at the site of the native mitral valve that is being replaced. In practice this means that in some embodiments, the inflow clamp jaws 102 will be long enough to almost touch the inside wall of the left atrium and ventricular clamp jaws 103 will be long enough to almost touch the inside wall of the left ventricle. In other embodiments, the inflow clamp jaws 102 will have a length that extends in the radial direction to a distance that is 50% of the distance from the dock stent to the inside wall of the left atrium and ventricular clamp jaws 103 will have a length that extends in the radial direction to a distance that is 50% of the distance from the dock stent to the inside wall of the left ventricle. Other embodiments that have intermediate lengths are also contemplated. Further, as the valve dock is deployed at the site of the native mitral valve, the ventricular clamp jaws will engage the native mitral valve leaflets pushing them up. The atraumatic design of the ventricular clamp jaws, such as of the clamp jaws 103 shown in FIG. 1A where the ends of the clamp jaws are curved away from the ventricular surface of the native leaflets, allows the valve dock to be deployed without injuring the native leaflets or the wall of the left ventricle.

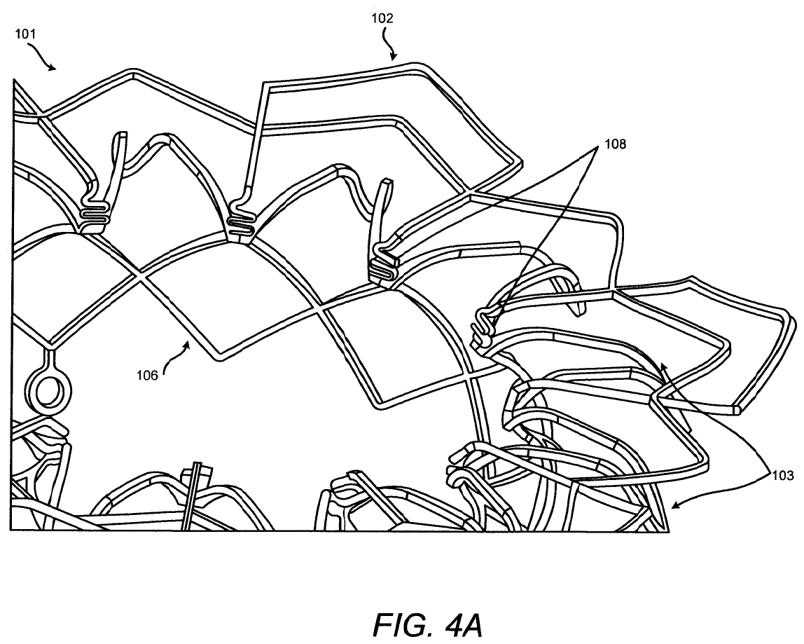

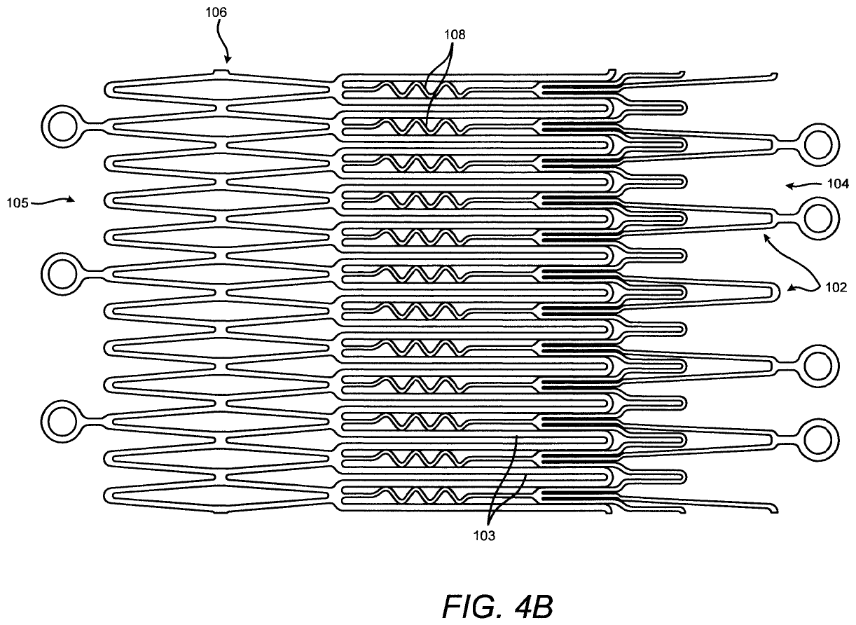

In some embodiments, as shown in FIG. 1C and FIG. 4A, the inflow clamp jaws 102 are connected to the dock stent via a strut that includes a strain relieving feature 108 discussed further hereinbelow. In one embodiment, the strain relieving feature 108 is shown in FIG. 4B, which is a two-dimensional pattern of the valve dock illustrating that the dock stent, the ventricular clamp jaws and the inflow clamp jaws can be cut continuously from a single tube. The strain relieving feature comprises a wiggle or serpentine strut, which allows the valve dock to be flexible along the radial direction while retaining rigidity along the longitudinal axis of the valve dock. Other benefits of this feature include, inter alfa, the following: (i) it allows the valve dock to be crimped to its delivery state within a delivery sheath with a lower radial force, (ii) it allows the outflow end to be more fully expanded while the inflow end is still crimped in the delivery sheath and (iii) it decouples the outflow end deformation from the inflow end deformation.

Valve dock 101 can be movable between a delivery configuration (not shown), a shape set configuration (FIG. 1A), and a deployed configuration (FIGS. 8B-8D). In the delivery configuration, valve dock 101 has a low profile suitable for delivery through small-diameter catheters positioned in the heart via the trans-septal, retrograde, or trans-apical approaches described hereinabove. In some embodiments, the delivery configuration of valve dock 101 will preferably have an outer diameter no larger than about 6-14 mm for trans-septal approaches, about 6-14 mm for retrograde approaches, or about 6-16 mm for trans-apical approaches to the native mitral valve. As used herein, “expanded configuration” refers to the configuration of the device (i) when allowed to freely expand to an unrestrained shape set size without the presence of constraining or distorting forces when the dock stent is self-expanding, and (ii) when the device is expanded to its larger size by applying pressure on the inside of the dock stent via, for example, an inflatable balloon. “Deployed configuration,” as used herein, refers to the device once expanded at the native valve site, engaging components of the native anatomy such as native mitral valve leaflets and the native mitral valve annulus for implantation at or adjacent to the native mitral valve annulus.

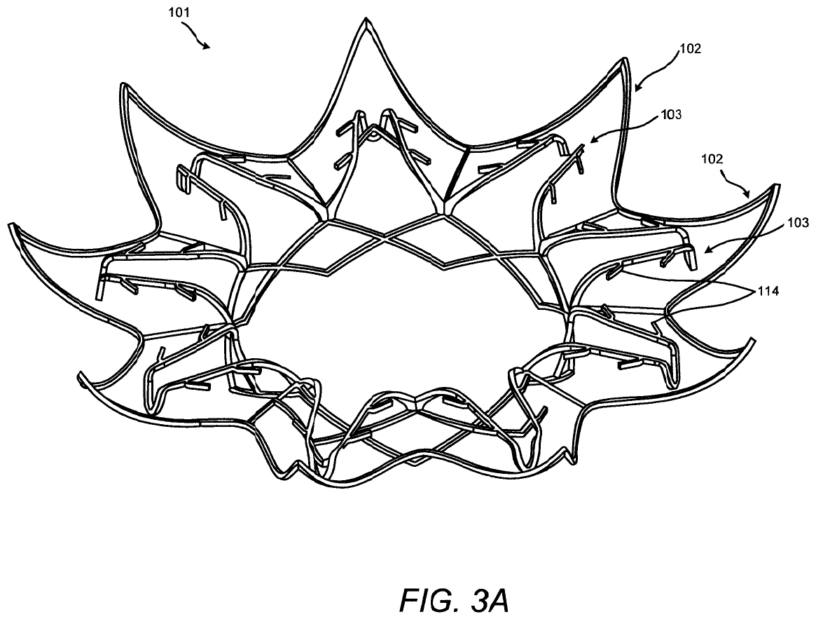

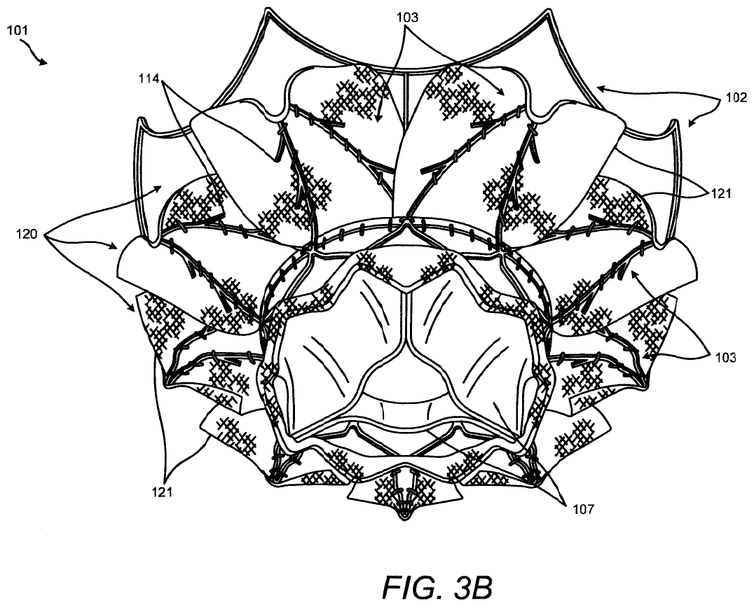

In another embodiment of valve dock 101 shown in FIG. 3A, ventricular clamp jaws 103 are further provided with one or more barbs 114. As will be discussed hereinbelow, barbs 114 facilitate capture of the native chordae tendineae during one approach to deployment of valve dock 101 at or adjacent to native mitral valve annulus.

In another embodiment of valve dock 101 shown in FIG. 3B, fabric pieces 120 are sutured to the legs of ventricular clamp jaws 103. Fabric pieces 120 have free edges 121 and fabric pieces of adjacent ventricular clamp jaws 103 overlap each other such that during deployment of valve dock 101 when ventricular clamp jaws 103 have been released from under the sheath of the delivery catheter, as discussed hereinbelow, chordae tendinae CT of the native mitral valve can slip in between free edges 121 and be captured by ventricular clamp jaws 103 and/or barbs 114. Although the inflow clamp jaws in FIG. 3B are not shown as being covered by fabric, in some embodiments, the inflow clamp jaws will be covered by fabric. The fabric covering promotes endothelization around the clamp jaws and in various embodiments described herein such a fabric cover can be put on the clamp jaws of the embodiments described herein, including, ventricular clamp jaws, inflow clamp jaws and atrial clamp jaws.

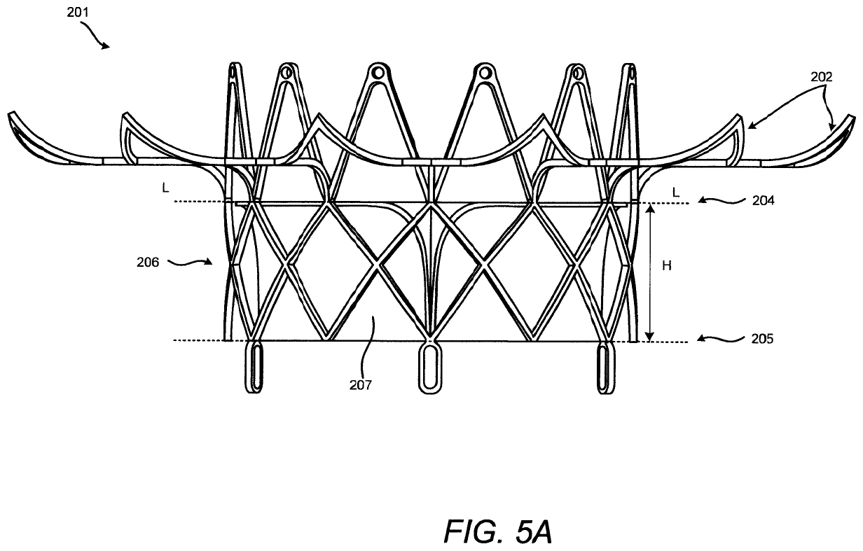

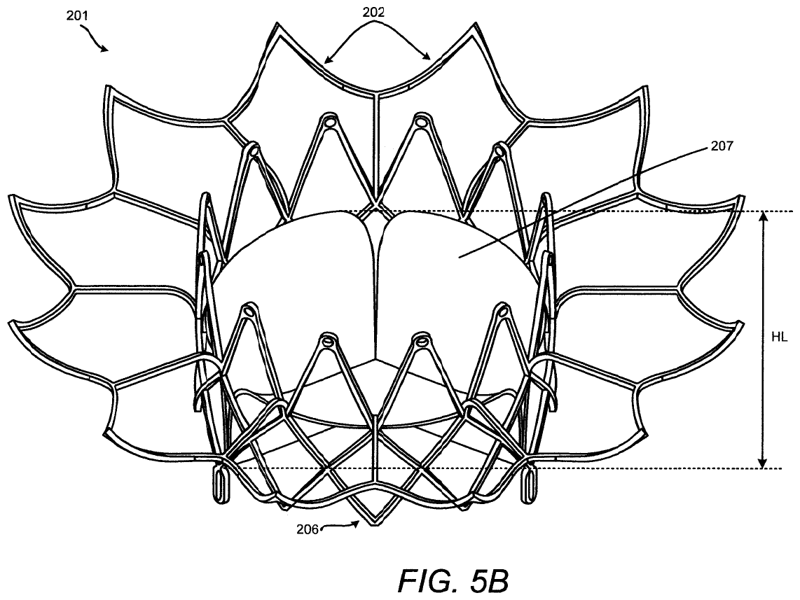

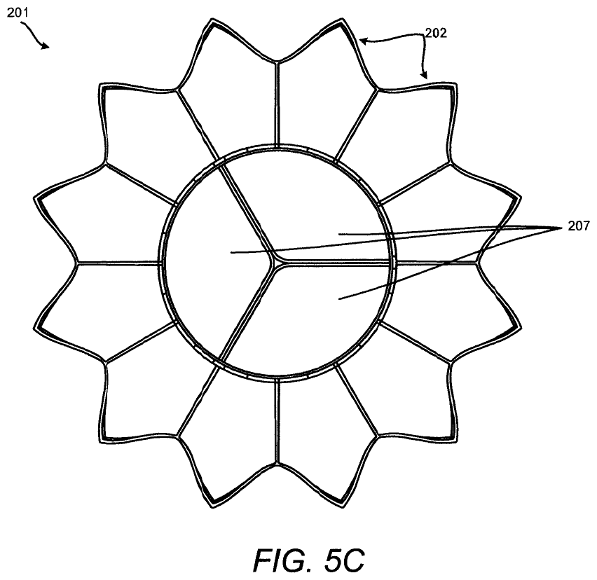

FIGS. 5A-5C show isometric views of a prosthetic mitral valve 201 in in its shape set configuration in accordance with an embodiment of the present technology. To replace the diseased or malfunctioning native mitral valve of a patient, prosthetic mitral valve 201 is docked inside valve dock 101 during deployment at or adjacent to the native mitral valve annulus of the patient.

As shown in FIG. 5A, the prosthetic mitral valve includes a valve stent 206 which has an inflow end 204 which is the end that would be nearest to the left atrium of the patient's heart post implantation of the prosthetic mitral valve and an outflow end 205 which would be the end furthest from the left atrium of the patient's heart post implantation of the prosthetic mitral valve. As shown in FIG. 5A, connected at or adjacent to inflow end 204 of valve stent 206 are one or more atrial clamp jaws 202.

In some embodiments, the length of valve stent 206 is minimized so that the valve stent does not extend so far into the left ventricle post implantation as to obstruct the LVOT. This is achieved in some embodiments by setting the height H of the valve stent below a certain amount. Height H of the valve stent is defined as the longitudinal distance from line LL (or from the inflow plane of the valve stent) to the distal (or outflow) end of the valve stent not including the eyelets on the outflow end of the valve stent as shown in FIG. 5A. In some embodiments, height H is <15 mm. In yet other embodiments, height H is <10 mm and in still other embodiments height H<6 mm. Although not shown with respect to other embodiments discussed hereinbelow, the height of the valve stent as measured from the inflow plane to the distal end, not including the eyelets, can be limited to <15 mm, or to <10 mm, or to <6 mm, to other embodiments of prosthetic mitral valves discussed hereinbelow.

Furthermore, in some embodiments, the extent to which prosthetic mitral valve leaflets extend into the left atrium is also limited. This is achieved in some embodiments by limiting the maximum longitudinal length of the leaflets HL defined as the maximum longitudinal length of the prosthetic mitral valve leaflets measured from the commissures to the cusps of the prosthetic mitral valve leaflets as illustrated in FIG. 5B. In some embodiments, HL−H<25 mm. In yet other embodiments, HL−H<20 mm and in still other embodiments HL−H<10 mm and in still other embodiments HL=H. When HL=H, the prosthetic mitral valve leaflets will sub-annulur which means that substantially the entire length of the prosthetic mitral valve leaflets will lie below the native mitral valve annulus post-implantation. Although not shown with respect to other embodiments discussed hereinbelow, the maximum longitudinal length of the prosthetic mitral valve leaflets can be limited as discussed hereinabove for other embodiments of prosthetic mitral valves discussed hereinbelow.

To regulate blood flow, the prosthetic mitral valve 201 also includes prosthetic leaflets 207 shown in FIGS. 5A-5C. Thus, the prosthetic mitral valve 201 comprises a plurality of prosthetic leaflets 207 supported by and within the valve stent 206. The plurality of prosthetic leaflets 207 and concomitant structure serve to regulate blood flow through the prosthetic mitral valve. The prosthetic leaflets 207 can comprise materials, such as bovine or porcine pericardial tissue or synthetic materials. The prosthetic leaflets 207 can be mounted to the valve stent 206 using well-known techniques and mechanisms. For example, the prosthetic leaflets 207 can be sutured to valve stent 206 in a tricuspid arrangement, as shown in FIG. 5C.

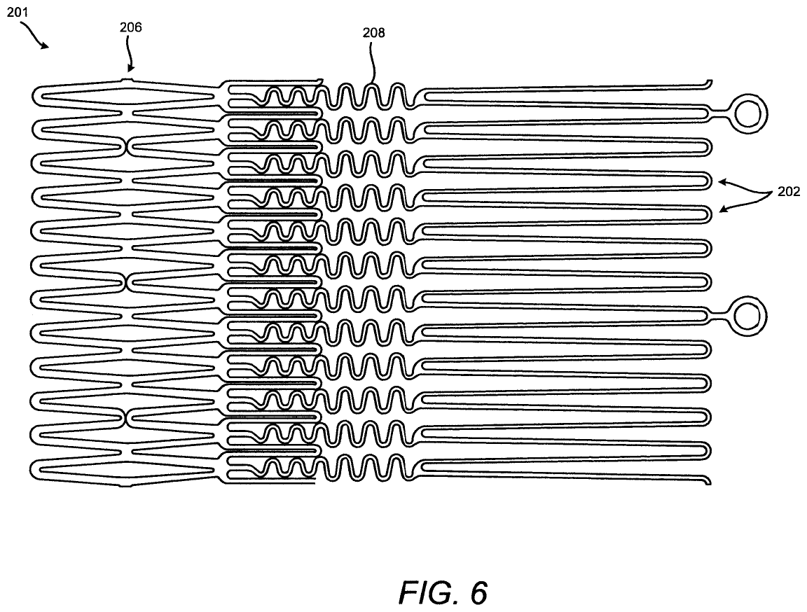

In some embodiments, the atrial clamp jaws 202 are connected to the valve stent via a strut that includes a strain relieving feature 208 discussed further hereinbelow. In one embodiment, the strain relieving feature 208 is shown in FIG. 6, which is a two-dimensional pattern of the prosthetic mitral valve 201 illustrating that the valve stent 206, and the atrial clamp jaws 202 can be cut continuously from a single tube. The strain relieving feature comprises a wiggle or serpentine strut, which allows the prosthetic mitral valve to be flexible along the radial direction while retaining rigidity along the longitudinal axis of the valve dock. Other benefits of this feature include, inter alia, the following: (i) it allows the prosthetic mitral valve to be crimped to its delivery state within a delivery sheath with a lower radial force, (ii) it allows the outflow end to be more fully expanded while the inflow end is still crimped in the delivery sheath and (iii) it decouples the outflow end deformation from the inflow end deformation. FIG. 6 also illustrates the alignment of the components of the prosthetic mitral valve when it is crimped onto a catheter under a sheath for deployment: atrial clamp jaws 202 are located proximally on the catheter whereas the valve stent 206 is located distally, which means that in the crimped state these two components are aligned at about 180 degrees to each other.

The valve stent 206 can be a tubular structure made of, for example, a wire mesh and can be radially collapsible and expandable between a radially expanded state and a radially compressed state for delivery and implantation at or adjacent to a native mitral valve annulus. The wire mesh can include metal wires or struts arranged in a lattice pattern. Valve stent 206 can be made of a shape-memory material, for example Nitinol, which makes the stent self-expandible from a radially compressed state to an expanded state. Alternatively, valve stent 206 can be plastically expandable from a radially compressed state to an expanded state using, for example, an inflatable balloon. Exemplary materials for such balloon expandable stents are stainless steel, chromium alloys, and/or other materials known to persons of ordinary skill in the art.

Valve stent 206 of prosthetic mitral valve 201 is sized such that when it is deployed in its expanded state inside valve dock 101, the outside surface of valve stent 206 pushes against the inside surface of dock stent 106 forming a frictional or interference fit with dock stent 106, thereby securing prosthetic mitral valve 201 inside valve dock 101. In some embodiments, valve stent 206 can be secured inside dock stent 106 using a lock and key approach (not shown) or other similar approaches for securing one stent member inside another.

Further, during deployment expansion of valve stent 206 inside dock stent 106, the outside surface of valve stent 206 pushes sacrificial prosthetic leaflets 107 against the inside surface of dock stent 106, sandwiching the leaflets between the two stents, whereby the sacrificial prosthetic leaflets cover the outside of the prosthetic mitral valve when it is implanted at or adjacent to the native mitral valve annulus. By thus covering the outside surface of the prosthetic mitral valve, the sacrificial leaflets serve as a seal to prevent paravalvular leakage.

In some embodiments, to replace the diseased or malfunctioning native mitral valve of a patient, a prosthetic mitral valve is docked inside valve dock 101 during deployment at or adjacent to the native mitral valve annulus of the patient. In one embodiment, prosthetic mitral valve 201 shown in FIGS. 5A-5C, discussed hereinabove, is docked inside valve dock 101 during deployment at or adjacent to the native mitral valve annulus of the patient.

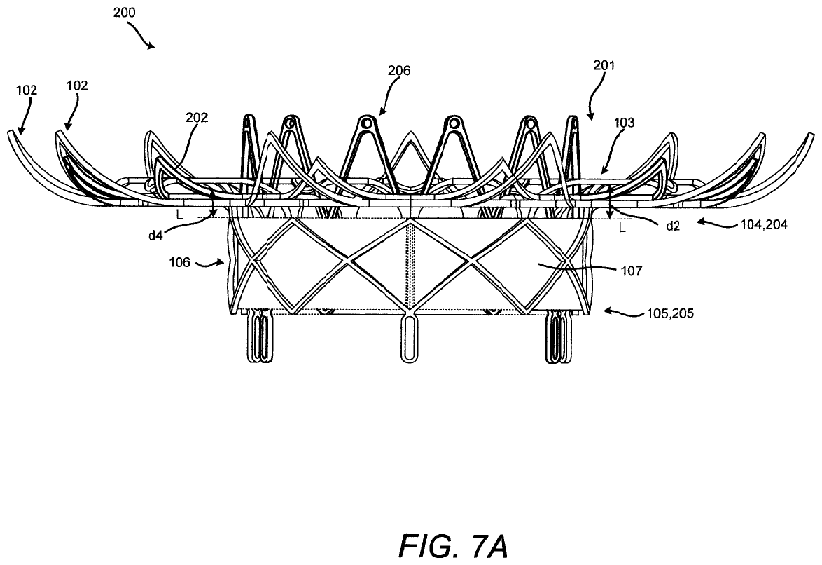

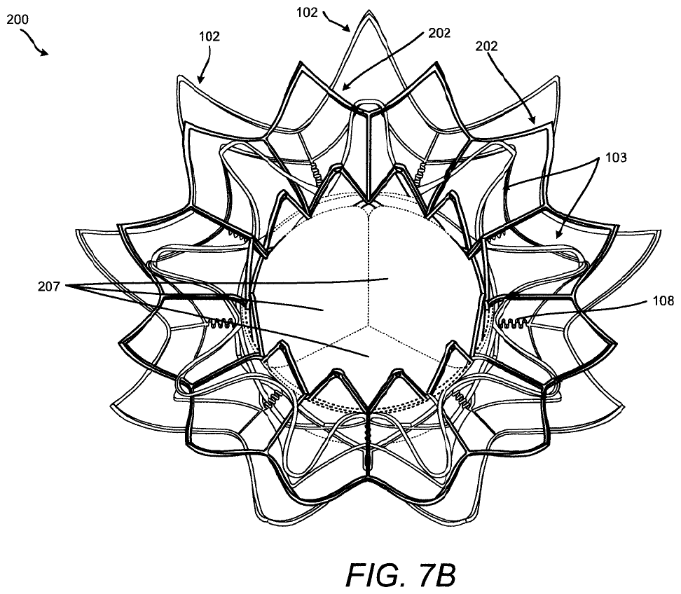

FIGS. 7A-7B show in a shape set configuration prosthetic mitral valve 201 anchored inside valve dock 101 to form shape set prosthetic mitral valve system 200. As shown in these figures, in the shape set configuration of prosthetic mitral valve system 200, the atrial clamp jaws 202 of prosthetic mitral valve 201 lie upstream or on the atrial side of the inflow clamp jaws 102 of valve dock 101 but ventricular clamp jaws 103 lie upstream of both inflow clamp jaws 102 and atrial clamp jaws 202. Upon deployment, as discussed below, because both atrial clamp jaws 202 and inflow clamp jaws 102 will be resiliently biased with respect to ventricular clamp jaws 103, in addition to inflow clamp jaws 102 atrial clamp jaws 202 will also act to clamp or pinch native mitral valve leaflets and native mitral annulus between them and ventricular clamp jaws 103. This feature allows the inflow clamp jaws of the valve dock to be designed such that they are thinner than the atrial clamp jaws of the valve dock, yielding a lower profile for the valve dock than would otherwise be possible. As shown in FIG. 7A, when prosthetic mitral valve 201 is anchored inside valve dock 101, sacrificial prosthetic leaflets 107 are pushed against the inside surface of dock stent 106, thereby providing a cover on the outside surface of valve stent 206 of prosthetic mitral valve 201, which helps to alleviate paravalvular leakage.

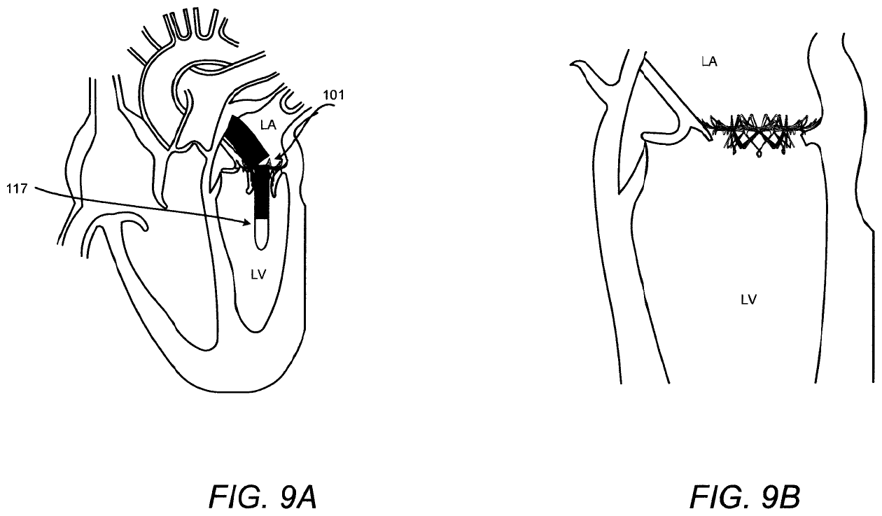

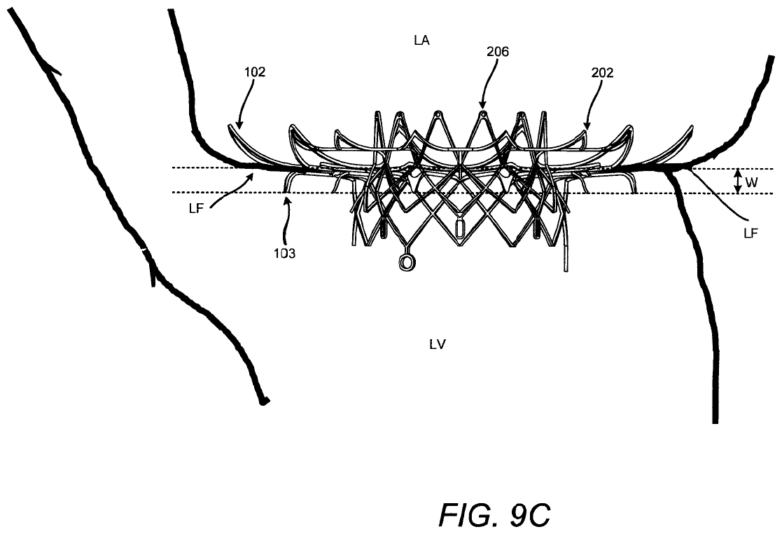

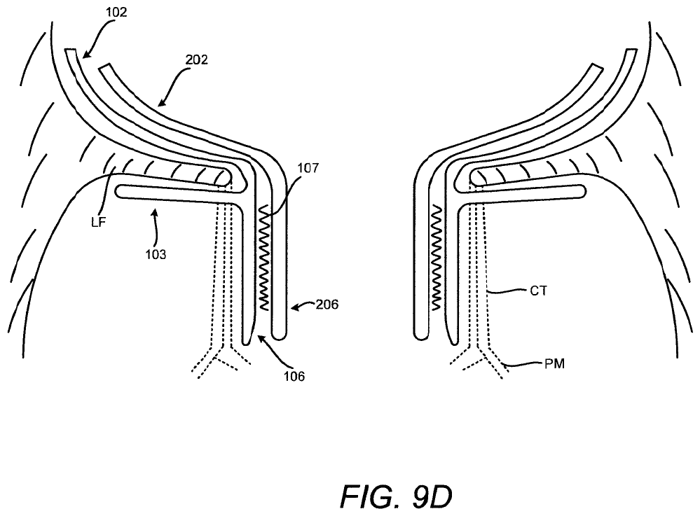

The relative location of atrial clamp jaws 202 and ventricular clamp jaws 103 in the shape set configuration of prosthetic mitral valve system 200 is such that when the valve dock is implanted at or adjacent to the native mitral valve annulus of a patient's heart, the two clamp jaws will act as a pair of forceps to clamp or pinch between them the native mitral valve leaflets and native mitral valve annulus as shown in FIGS. 9C and 9D, and discussed hereinbelow. As shown in FIG. 7A, in the shape set configuration of prosthetic mitral valve system 200, and, therefore, in the shape set configurations of valve dock 101, and prosthetic mitral valve 201, a portion of the legs of ventricular clamp jaws 103 lies upstream of a portion of the legs of atrial clamp jaws 202. As will be discussed below, when the valve dock is deployed at or adjacent to the native mitral valve annulus, ventricular clamp jaws 103 will be positioned on the ventricular or downstream side of the native mitral valve annulus and the atrial clamp jaws 202 will be positioned on the atrial or upstream side of the native mitral valve annulus, with the native mitral valve leaflets and native mitral valve annulus sandwiched between them, which means that the two sets of clamp jaws have been separated apart with their positions switched and, therefore, have been elastically deformed from their state as part of the shape set prosthetic mitral valve system. Accordingly, they will be resiliently biased to return to their shape state, wherein the ventricular clamp jaws are upstream of the atrial clamp jaws compared to their deployed states, wherein atrial clamp jaws are upstream of the ventricular clamp jaws, thereby pinching or clamping the native mitral valve leaflets and native mitral annulus between them.

The relative location of atrial clamp jaws and the ventricular clamp jaws in the shape set configuration of prosthetic mitral valve system can be illustrated with reference to the embodiment shown in FIG. 7A, where line LL is perpendicular to the longitudinal axis of dock stent 106 and lies in the inflow plane of the dock stent. Longitudinal distance d from line LL to any point is defined as the distance measured from line LL to that point along a line that is perpendicular to line LL. If the point lies upstream of line LL (or proximally of line LL), d will be positive. For points that lie downstream of line LL (or distally of line LL), distance d will be negative.

As can be seen with reference to FIG. 7A, the legs of the “U” shaped ventricular clamp jaws 103 have a section that is substantially parallel to line LL and furthest from line LL. Therefore, d2, the longitudinal distance between any point on this section and line LL, is the maximum longitudinal distance between any point on ventricular clamp jaws 103 and line LL. Similarly, the legs of the “V” shaped atrial clamp jaws 202 have a section that is substantially parallel to line LL and nearest to line LL. Therefore, d4, the longitudinal distance between any point on this section and line LL is the minimum longitudinal distance between any point on atrial clamp jaws 202 and line LL. As illustrated here distances d4 and d2 are measured from line LL to sections of jaws 202 and 103, respectively, that are substantially parallel to line LL, and in general, they are defined as: d4 is the minimum longitudinal distance between atrial clamp jaw 202 and line LL and d2 is the maximum longitudinal distance between ventricular clamp jaws 103 and line LL.

If we define maximum relative separation d5 as being equal to d2−d4, in the embodiment shown in FIG. 7A, i.e., in the shape set configuration of the prosthetic mitral valve system shown in this figure, d5>0 because ventricular clamp jaws 103 are upstream of atrial clamp jaws 202. If d5>0, in the shape set configuration of the prosthetic mitral valve system 200, some portion of ventricular clamp jaws 103 lies further upstream (or further in the proximal direction) from line LL than some portion of atrial clamp jaws 202. In that case, because upon deployment the atrial clamp jaws are on the atrial side of the native mitral valve annulus and ventricular clamp jaws are on the ventricular side of the native mitral valve annulus, all of the atrial clamp jaws will be further upstream (or further in the atrial direction) than the ventricular clamp jaws and the native mitral valve leaflets and the native mitral valve annulus will be in between the two jaws, which means that the clamp jaws are elastically deformed from their shape set configuration. This means that the ventricular clamp jaws and atrial clamp jaws are resiliently biased with respect to each other and will have a tendency to move relative to each other to try to restore the shape set configuration of prosthetic mitral valve system 200. However, because deployment of valve dock 101 and docking of prosthetic mitral valve 201 inside the deployed valve dock 101 is done in such a way that the native mitral valve leaflets and the native mitral valve annulus is trapped between them, the resilient forces between the two sets of clamp jaws will result in the native mitral valve leaflets and native mitral valve annulus being pinched or clamped between them.

In the embodiments shown in FIGS. 7A, d2>d4 and, therefore, d5>0 and as discussed above this configuration of the ventricular and atrial clamp jaws will have the effect of these two sets of clamp jaws being resiliently biased with respect to each other when the valve dock and the prosthetic mitral valve are deployed, which will cause the native mitral valve leaflets and mitral valve annulus to be pinched or clamped between the ventricular and atrial clamp jaws. This effect will obtain if d5>0. In other embodiments, d2 may be equal to or even be less than d4 as long as the atrial clamp jaws and ventricular clamp jaws are sufficiently close to each other so that upon deployment of valve dock 101 and prosthetic mitral valve 202, native mitral valve leaflets LF are pressed between the clamp jaws 202 and 103. In general, given the anatomy of the native mitral valve, if d5>−4 mm (i.e., d5 measured in millimeters is greater than minus 4), ventricular clamp jaws 103 and atrial clamp jaws 202 upon deployment, i.e., when ventricular clamp jaws are downstream of the native annulus and atrial clamp jaws are upstream of the native annulus, will be sufficiently resiliently biased to pinch or clamp the native mitral valve leaflets and native mitral valve annulus between them.

In the embodiment shown in FIGS. 7A, sections of both the “U” shaped ventricular clamp jaws and the “V” shaped atrial clamp jaws are substantially parallel to line LL, these sections are substantially parallel to each other and, therefore, the legs of the “U” shaped ventricular clamp jaws have a section that is substantially parallel to a section of the legs of the “V” shaped atrial clamp jaws. In other embodiments, atrial clamp jaws 202 and ventricular clamp jaws 103 can be such that they do not have any sections that are parallel to each other or to line LL. In such embodiments, clamp jaws 202 and 103 would be resiliently biased so that the native mitral valve leaflets and the native mitral annulus is clamped between them if d5>−4 mm.

Although not shown, in the deployed state when prosthetic mitral valve 201 is anchored inside valve dock 101, sacrificial prosthetic leaflets are pushed against the inside surface of dock stent 106, thereby providing a cover on the outside surface of valve stent 206 of prosthetic mitral valve 201, which helps to alleviate paravalvular leakage (similar to prosthetic mitral valve system 200 shown in FIG. 7A). Upon deployment of the prosthetic mitral valve 201 at or adjacent to the native mitral valve annulus of a patient, prosthetic leaflets 207, shown in FIG. 7A, serve to regulate blood flow between the left atrium and left ventricle of the patient.

Prosthetic mitral valve 201 can be movable between a delivery configuration (not shown), a shape set configuration (FIGS. 5A-5C), and a deployed configuration (FIGS. 9B-9D). In the delivery configuration, prosthetic mitral valve 201 has a low profile suitable for delivery through small-diameter catheters positioned in the heart via the trans-septal, retrograde, or trans-apical approaches described hereinabove. In some embodiments, the delivery configuration of prosthetic mitral valve 201 will preferably have an outer diameter no larger than about 6-14 mm for trans-septal approaches, about 6-14 mm for retrograde approaches, or about 6-16 mm for trans-apical approaches to the native mitral valve.

In treating a patient suffering from mitral valve regurgitation or other mitral valve insufficiency, valve dock 101 and prosthetic mitral valve 201 are deployed at or adjacent to the native mitral valve annulus such that prosthetic mitral valve 201 is deployed inside valve dock 101. FIGS. 8A-8D show the deployment of valve dock 101 at or adjacent to the native mitral valve annulus. As discussed before, after percutaneously accessing the femoral vein, a catheter having a needle or a guidewire is advanced into the right atrium RA through the inferior vena cava IVC. When the catheter is on the anterior side of the inter-atrial septum, the needle or guidewire is made to penetrate the inter-atrial septum. Following this, in the case where a needle is used instead of a guidewire, a guidewire is exchanged for the needle and the catheter is withdrawn. By placing a catheter over the guidewire access to the left atrium through the inter-atrial septum is maintained. The catheter can then be used to deliver the prosthetic mitral valve system at or adjacent to the native mitral valve annulus. In the illustrated embodiment, valve dock 101 (not shown) is crimped to a collapsed condition under sheath 116 of catheter 115. Valve dock 101 is crimped so that the inflow clamp jaws and ventricular clamp jaws are parallel to the longitudinal axis with the clamp jaws bent in an upwards direction, i.e., the direction that is opposite to the direction of the longitudinal axis of dock stent 106 from inflow end 104 to outflow end 105. To illustrate, inflow and ventricular clamp jaws, 102 and 103, respectively, of the valve dock 101 will be aligned with respect to the rest of the dock stent as shown in FIG. 4B, i.e., the angle between inflow and ventricular clamp jaws, 102 and 103, and dock stent 106 will be about 180 degrees. Furthermore, in the crimped state, inflow and ventricular clamp jaws, 102 and 103, respectively, referring to FIG. 8A, will be nearer to the proximal end of catheter 115 than dock stent 106 is to the proximal end of catheter 115, and, therefore, dock stent 106 will be nearer to the distal end of catheter 115 compared to the distance of inflow and ventricular clamp jaws from the distal end.

During deployment, catheter 115 with the crimped valve dock under the catheter's sheath is advanced into the left ventricle just past the free edge of at least one of the native mitral valve leaflets. In that position, sheath 116 of catheter 115 is partially withdrawn proximally first releasing dock stent 106. Once proper positioning of dock stent clear of the native mitral valve leaflets has been verified, sheath 116 is further withdrawn proximally, releasing ventricular clamp jaws 103. On being released ventricular clamp jaws 103 will tend to assume their expanded, shape set configuration, i.e., they will extend radially outwards from the inflow end of dock stent 106 towards the inside wall of the left ventricle so that they are arranged around the circumference of dock stent 106 and extend radially from dock stent 106. With the ventricular clamp jaws thus released, catheter 115 is pulled proximally which pulls ventricular clamp jaws towards the native mitral valve annulus which, in turn, push the native mitral valve leaflets towards the native mitral valve annulus till the leaflets are crushed or pressed between the ventricular side of the native mitral valve annulus and ventricular clamp jaws 103.

Once catheter 115 has been pulled back sufficiently to trap and tightly press the native mitral valve leaflets between ventricular jaws 103 and the ventricular side of the native mitral valve annulus, two different approaches to deploying the rest of valve dock 101 can be used: in a first approach, catheter 115 is rotated prior to deploying inflow clamp jaws 102, and in a second approach catheter 115 is not rotated prior to deploying inflow clamp jaws 102.

In the first approach, once the leaflets have been trapped between ventricular clamp jaws 103 and the native mitral valve annulus, catheter 115 is rotated axially so that dock stent 106 and, therefore, ventricular clamp jaws 103 are rotated axially to entangle and/or capture the native chordae tendineae. This results in the native leaflets being more securely and uniformly spread out underneath the native mitral valve annulus and around ventricular clamp jaws 103. In one embodiment of this approach, valve dock 101 as shown in FIG. 3A is used instead of valve dock 101 shown in FIG. 1A. When ventricular clamp jaws 103 are rotated by rotating catheter 115, one of more barbs 114 facilitate capture of the native chordae tendineae by preventing the chordae from slipping off ventricular clamp jaws 103. Once the leaflets have been thus secured between ventricular clamp jaws 103 and the native mitral valve annulus and chordae have been captured by ventricular clamp jaws 103, catheter 115 is pulled back proximally until the distal end of sheath 116 is on the atrial side of the native mitral valve annulus. Once this position has been confirmed, sheath 116 is withdrawn proximally to release inflow clamp jaws 102.

In the second approach, once the leaflets have been trapped between ventricular clamp jaws 103 and the native mitral valve annulus, catheter 115 is not rotated but is simply pulled back proximally until the distal end of sheath 116 is on the atrial side of the native mitral valve annulus. Once this position has been confirmed, sheath 116 is withdrawn proximally to release inflow clamp jaws 102.

Under both approaches, on being released, inflow clamp jaws 102 will tend to assume their shape set configuration which means that they will bend down towards the distal end of catheter 115 until at least some part of inflow clamp jaws 102 press down on the atrial side of the native mitral valve annulus as shown in FIGS. 8C and 8D. Because the ends of inflow clamp jaws 102 are bent up towards the proximal end of catheter 115, inflow clamp jaws move towards their expanded shape set configuration atraumatically without injuring the inside wall of the left atrium.

At this point valve dock 101 is fully deployed at or adjacent to the native mitral valve annulus as shown in FIGS. 8C-8D. Inflow clamp jaws 102 are seated on the atrial side of the native mitral valve annulus inside the left atrium, ventricular clamp jaws 103 are seated on the ventricular side of the native mitral valve annulus, and leaflets LF are sandwiched between inflow clamp jaws 102 and ventricular clamp jaws 103.

As shown in FIG. 8D, in the deployed state of valve dock 101, ventricular clamp jaws 103 are on the ventricular side of mitral valve annulus whereas inflow clamp jaws 102 are on the atrial side of the mitral valve annulus which means that in the deployed state inflow clamp jaws 102 are further upstream than ventricular clamp jaws 103. However, as discussed above, in the shape set configuration of valve dock 101 shown in FIGS. 1-3, ventricular clamp jaws are further upstream than inflow clamp jaws. Therefore, upon deployment of valve dock 101, ventricular clamp jaws 103 and inflow clamp jaws 102 are deformed from their shape set configuration relative to each other and accordingly are resiliently biased. As discussed previously, this deformation results in a resilient force between them to try to restore their shape set configuration, the state as shown in FIG. 1A. This force acts to pinch or clamp the native mitral valve leaflets LF between inflow clamp jaws 102 and ventricular clamp jaws 103, thereby forming a secure anchor for valve dock 101 around the native mitral valve annulus.

In the embodiment shown in FIGS. 8C and 8D, leaflets LF are crushed or pressed between inflow clamp jaws 102 and ventricular clamp jaws 103 such that most of the mass of leaflets lies in a narrow, confined space between the native mitral valve annulus and ventricular clamp jaws 103 and is further compressed between ventricular clamp jaws 103 and inflow clamp jaws 102. As discussed above, this is to be distinguished from prior art prosthetic mitral valve designs where to avoid injuring chordae tendineae the native mitral valve leaflets are more or less allowed to hang down into the left ventricle as they would be in the open state of the native mitral valve. Such designs tend to increase obstruction of the left ventricle outflow tract (LVOT), rendering such designs unacceptable for a vast number of patients suffering from mitral valve regurgitation or other insufficiency.

Following deployment and implantation of the valve dock, the over the wire catheter 115 for the valve dock is exchanged for another catheter that has crimped on it a prosthetic mitral valve. In one embodiment, prosthetic mitral valve 201 is crimped into a delivery configuration and delivered and deployed inside the implanted valve dock 101 at or adjacent to the native mitral valve annulus as shown in FIGS. 9A-9D.

In the illustrated embodiment, prosthetic mitral valve 201 is crimped to a collapsed condition (not shown) under sheath 118 on a catheter 117. Prosthetic mitral valve 201 is crimped so that atrial clamp jaws 202 are parallel to the longitudinal axis with the clamp jaws bent to point in a proximal direction. To illustrate, atrial clamp jaws 202 of prosthetic mitral valve 201 will be aligned with respect to valve stent 206 of prosthetic mitral valve 201 as shown in FIG. 6 as discussed hereinabove, i.e., the angle between the atrial clamp jaws 202 and valve stent 206 will be about 180 degrees. Thus, in the crimped state, atrial clamp jaws, 202 referring to FIG. 9A, will be nearer to the proximal end of catheter 117 than valve stent 206 is to the proximal end of catheter 117, and, therefore, valve stent 206 will be nearer to the distal end of catheter 117 compared to the distance of atrial clamp jaws from the distal end.

During deployment, catheter 117 with the crimped prosthetic mitral valve 201 under the catheter's sheath is advanced just past the native mitral valve annulus into the left ventricle LV pushing back the sacrificial prosthetic leaflets. In that position, sheath 118 of catheter 117 is partially withdrawn proximally first releasing valve stent 206 inside the already deployed dock stent 106. Valve stent 206 is secured inside dock stent 106 by friction fit, interference fit, a lock and key type of fit, or other such approaches for securing a cylindrical object inside another cylindrical object.

Once valve stent 206 has been properly secured inside dock stent 106, sheath 118 is further withdrawn proximally, releasing atrial clamp jaws 202.

On being released, atrial clamp jaws 202 will tend to assume their expanded state which means that they will bend down towards the distal end of catheter 117 until at least some part of atrial clamp jaws 202 press down on the atrial side of the native mitral valve annulus as shown in FIGS. 9C and 9D. Because the ends of atrial clamp jaws 202 are bent up towards the proximal end of catheter 117 and away from the inside wall of the left atrium, inflow clamp jaws move towards their expanded state atraumatically without injuring the inside wall of the left atrium.

At this point atrial clamp jaws 202 are seated just above previously implanted inflow clamp jaws 102 as shown in FIGS. 9C and 9D. Thus, leaflets LF are pinched or clamped between atrial clamp jaws 202, inflow clamp jaws 102, and ventricular clamp jaws 103 as shown in FIGS. 9C and 9D. In the embodiment shown in these figures, leaflets LF are sandwiched between atrial clamp jaws 202 and inflow clamp jaws 102 and ventricular clamp jaws 103 such that the leaflets LF are substantially pressed between atrial clamp jaws 202, inflow clamp jaws 102 and ventricular clamp jaws 103 and almost the entire mass of leaflets LF is closely confined in a narrow region around the native mitral valve annulus and between ventricular clamp jaws 103 and atrial clamp jaws 202. We can define this region as being bounded on the upstream side by the inflow plane of valve stent 206 and by a parallel plane on the downstream side where the longitudinal distance between the two planes and, therefore, the width of the region is W. In some embodiments, leaflets LF are substantially held in a region bounded by said two planes, where W is less than 4 mm. In other embodiments, W is less than 3 mm, and in still other embodiments W is less than 2 mm. As discussed above, this is to be distinguished from prior art prosthetic mitral valve designs where some part or all of the native mitral valve leaflets are allowed to remain in a direction that is more or less parallel to the longitudinal axis of the prior art prosthetic mitral valve or dock. Such designs tend to increase obstruction of the left ventricle outflow tract (LVOT), rendering such designs unacceptable for a vast number of patients suffering from mitral valve regurgitation or other insufficiency. Although the native mitral valve leaflets LF are pushed up to be held in the relatively narrow region bounded by atrial clamp jaws 202 and ventricular clamp jaws 103, leaflets LF continue to be attached to papillary muscles PM through tendinae chordae TC as shown in FIG. 9D.

In some embodiments, prosthetic mitral valve system 200 extends only a short distance downstream of the annulus into the left ventricle to limit obstruction of the LVOT. Therefore, in some embodiments of prosthetic mitral valve system, after deployment at or adjacent to the native mitral valve annulus, atrial and ventricular clamp jaws are separated by relatively short distance so that the structure formed by atrial and ventricular clamp jaws, 202 and 103, respectively, and native mitral valve leaflets LF do not extend into or obstruct the LVOT. This is achieved in some embodiments by making d5>0 (or generally, d5>−4 mm) in the shape set configuration of mitral valve system 200, as shown in FIG. 7A and discussed above.

As discussed previously, the prosthetic mitral valve system in some embodiments can be delivered and deployed at or adjacent to the native mitral valve annulus using a retrograde approach to the mitral valve via the aorta and left ventricle.

In yet other embodiments, the prosthetic mitral valve system can be delivered and deployed at or adjacent to the native mitral valve annulus using a transapical approach. Thus, in an exemplary method, the valve dock is crimped under a sheath onto a custom-made 30F delivery device and then advanced through a 2-cm left apical incision into the left ventricle. Then, in a stepwise process, the inflow clamp jaws and ventricular clamp jaws of the valve dock are released so as to capture the native mitral valve leaflets. The sheath is then slowly withdrawn, releasing the dock stent. The first delivery device is removed and a second delivery device is introduced that has the prosthetic mitral valve crimped under a sheath mounted on the delivery device. The second delivery device is then advanced to a point just past the inflow clamp jaws of the implanted valve dock. The sheath is then partially withdrawn to release the atrial clamp jaws of the prosthetic mitral valve. Once the atrial clamp jaws are placed about the inflow clamp jaws of the valve dock, the sheath is further withdrawn to release the valve stent, which then expands to form a, for example, friction fit with the dock stent. Once appropriate positioning is confirmed, the delivery device is removed.

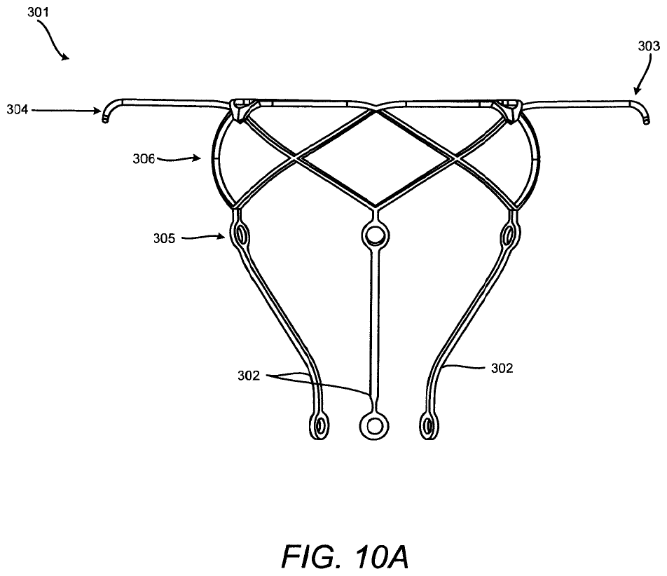

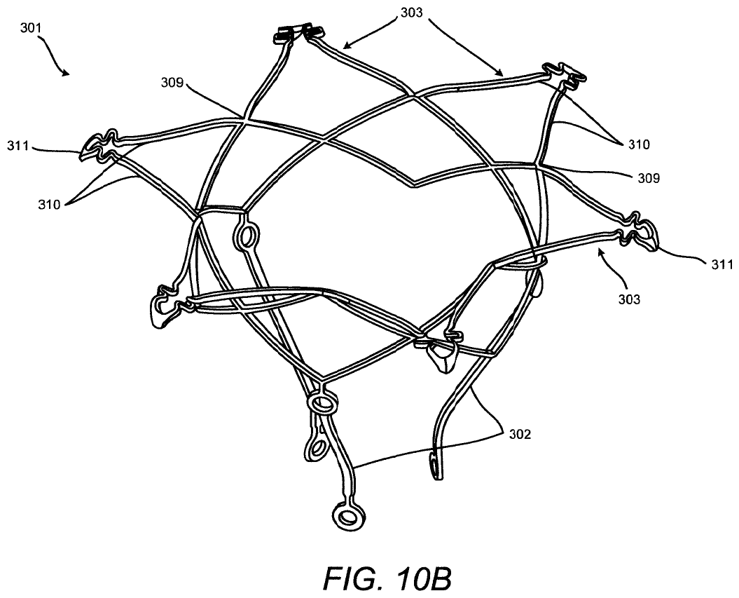

In another embodiment, the prosthetic mitral valve system comprises valve dock 301 as shown in FIGS. 10A and 10B. FIGS. 10A-10B show isometric views of a valve dock 301 in a shape set configuration in accordance with an embodiment of the present technology. As shown in FIG. 10A, valve dock 301 includes a dock stent 306 which has an inflow end 304 which is the end that would be nearest to the left atrium of the patient's heart post implantation of the valve dock and an outflow end 305 which would be the end furthest from the left atrium of the patient's heart post implantation of the valve dock. Thus, blood flows from the left atrium into the inflow end and flows out of the outflow end into the left ventricle.

Dock stent 306 can be a tubular structure made of, for example, a wire mesh and can be radially collapsible and expandable between a radially expanded state and a radially compressed state for delivery and implantation at or adjacent to a native mitral valve annulus. The wire mesh can include metal wires or struts arranged in a lattice pattern. Dock stent 306 can be made of a shape-memory material, for example Nitinol, which makes the dock stent self-expandable from a radially compressed state to an expanded state. Alternatively, the dock stent 306 can be plastically expandable from a radially compressed state to an expanded state using, for example, an inflatable balloon. Exemplary materials for such balloon expandable dock stents can be stainless steel, chromium alloys, and/or other materials known to persons of ordinary skill in the art.

In the interim period between implantation of valve dock 301 and implantation of the prosthetic mitral valve as discussed below, the native mitral valve leaflets cannot regulate blood flow between the left atrium and the left ventricle. To regulate blood flow during this interim period, in one embodiment, the valve dock 301 also includes sacrificial prosthetic leaflets (not shown here but would be similar to sacrificial leaflets 107 shown in FIGS. 1B and 1C for valve dock 101). Thus, the valve dock 301 comprises a plurality of sacrificial prosthetic leaflets supported by and within the dock stent 306. The plurality of sacrificial prosthetic leaflets and concomitant structure serves to regulate blood flow through the valve dock prior to implantation of a prosthetic mitral valve. The sacrificial prosthetic leaflets can comprise materials, such as bovine or porcine pericardial tissue or synthetic materials. The sacrificial prosthetic leaflets can be mounted to the dock stent 306 using well-known techniques and mechanisms. For example, the sacrificial prosthetic leaflets can be sutured to the dock stent 306 in a tricuspid arrangement (not shown here but would be similar to as shown in FIG. 1C with respect to sacrificial leaflets 107 for valve dock 101).

As will be discussed below, when a prosthetic mitral valve is implanted inside valve dock 301, sacrificial prosthetic leaflets will be pushed aside by the stent of the prosthetic mitral valve and will cover the outside surface of the valve stent of the prosthetic mitral valve, thereby acting as a barrier to paravalvular leak (PVL).

As shown in FIGS. 10A and 10B, connected at or adjacent to inflow end 304 of dock stent 306 are one or more ventricular clamp jaws 303. In the embodiment shown in FIG. 10B, there are 6 sets of ventricular clamp jaws 303, arranged equidistant from each other around inflow end 304. Thus, the vertices of adjacent ventricular clamp jaws 303 are 60 degrees apart from each other. In various embodiments, ventricular clamp jaws 303 can be evenly spaced from each other, where adjacent clamp jaws are from 20-180 degrees apart. In other embodiments, ventricular clamp jaws 303 can be unevenly spaced from each other. Thus, for example, one set of adjacent clamp jaws can be 20 degrees apart whereas another adjacent set can be 60 degrees apart.

In one embodiment, one or more anchor legs 302 are connected to the outflow end 305 of dock stent 306 at points 312 as shown in FIGS. 10A and 10B. As will be discussed below, anchor legs 302 allow valve dock 301 to be securely held in position during deployment prior to deployment of the prosthetic mitral valve inside valve dock 301.

In one embodiment of a valve dock, one or more ventricular clamp jaws 303 is in the shape of a “U” with legs 310 and a valley end 311 as shown in FIG. 10B, which is a perspective view of valve dock 301. Legs 310 of the “U” flare outwards and connect with dock stent 306 at 309. As shown in FIG. 10A, legs 310 of “U” shaped ventricular clamp jaw 303 are bent such that valley end 311 points in the direction of the outflow end 305 of dock stent 306. In other words, in the deployed configuration of valve dock 301 valley end 311 will point away from the left atrium post implantation. The softly curved “U” shape of ventricular clamp jaw 303 with bent valley end 311 makes it atraumatic. Ventricular clamp jaws 303 may also be made atraumatic by wrapping the clamp jaws with tissue, such as bovine or porcine pericardium tissue, or by other materials such as polytetrafluoroethylene (PTFE).

In the embodiment shown in FIG. 10A, except for the bent valley end, a section of ventricular clamp jaws 303 is substantially perpendicular to the longitudinal axis of valve dock 301. With this feature, when the valve dock is deployed at or adjacent to the native mitral valve annulus, native mitral valve leaflets will be pressed between the ventricular clamp jaws and the native mitral valve annulus, and when ventricular clamp jaws are fully deployed a substantial portion of the leaflets is pressed into the native mitral valve annulus by that section of ventricular clamp jaws 303 which is generally perpendicular to the longitudinal axis of valve dock 301. In other embodiments, ventricular clamp jaws 303 may not have any section that is perpendicular to the longitudinal axis of valve dock 301 as long as ventricular clamp jaws 303 are designed such that on deployment they press the leaflets back against native mitral valve annulus at one or more points which lie in a plane that is generally orthogonal to the longitudinal axis of valve dock 301.

The radial lengths of the clamp jaws 303 should be sufficient to provide a stable and secure anchor for implanting the prosthetic mitral valve at the site of the native mitral valve that is being replaced. In practice this means that in some embodiments, the ventricular clamp jaws 303 will be long enough to almost touch the inside wall of the left ventricle. In other embodiments, the ventricular clamp jaws 303 will have a length that extends in a radial direction to a distance that is 50% of the distance from the dock stent to the inside wall of the left ventricle. Other embodiments that have intermediate lengths are also contemplated. The atraumatic design of the ventricular clamp jaws 303 where valley ends 311 are curved allows the valve dock to be deployed without injuring the wall of the left ventricle.

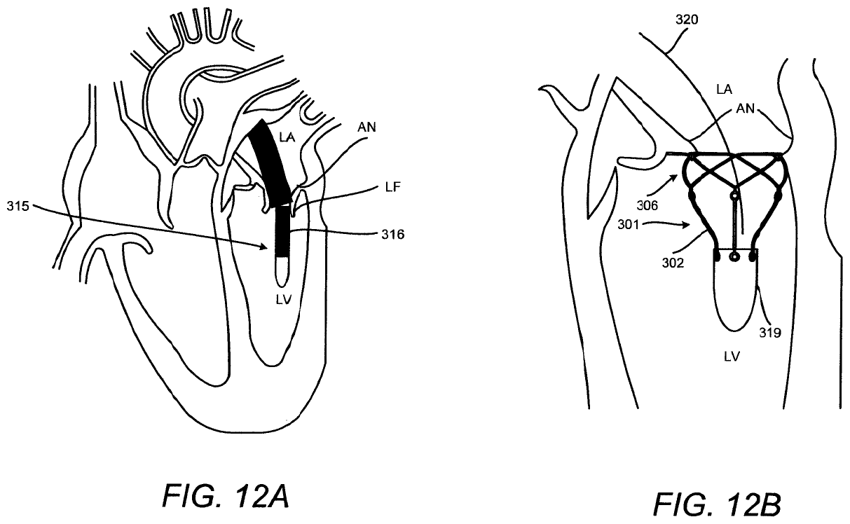

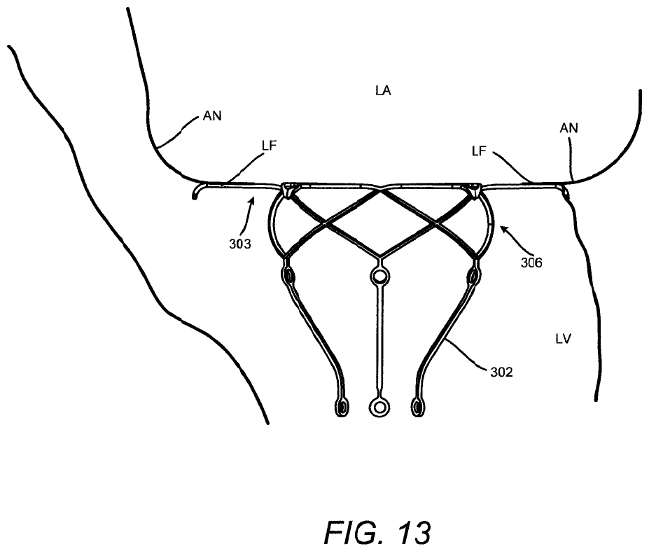

Valve dock 301 can be movable between a delivery configuration (not shown), a shape set configuration (FIG. 10A), and a deployed configuration (FIGS. 12B and 13). In the delivery configuration, valve dock 301 has a low profile suitable for delivery through small-diameter catheters positioned in the heart via the trans-septal, retrograde, or trans-apical approaches described hereinabove. In some embodiments, the delivery configuration of valve dock 301 will preferably have an outer diameter no larger than about 6-14 mm for trans-septal approaches, about 6-14 mm for retrograde approaches, or about 6-16 mm for trans-apical approaches to the native mitral valve.

In another embodiment of valve dock 301 (not shown) ventricular clamp jaws 303 are further provided with one or more barbs (similar to the barbs 114 on ventricular clamp jaws 101 shown in FIG. 3). As will be discussed hereinbelow, such barbs facilitate capture of the native chordae tendineae during one approach to deployment of valve dock 301 at or adjacent to native mitral valve annulus.

In some embodiments, to replace the diseased or malfunctioning native mitral valve of a patient, a prosthetic mitral valve is docked inside valve dock 301 during deployment at or adjacent to the native mitral valve annulus of the patient. In one embodiment, prosthetic mitral valve 201 shown in FIGS. 5A-5C, discussed hereinabove, is docked inside valve dock 301 during deployment at or adjacent to the native mitral valve annulus of the patient.

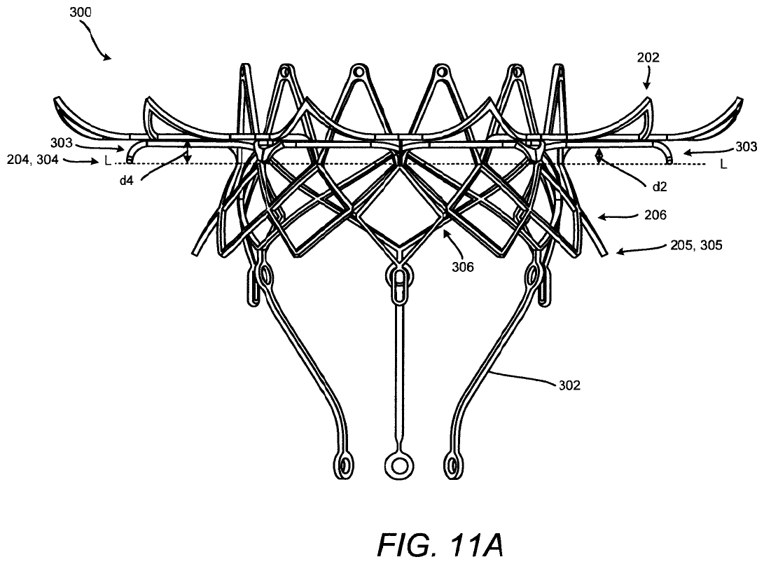

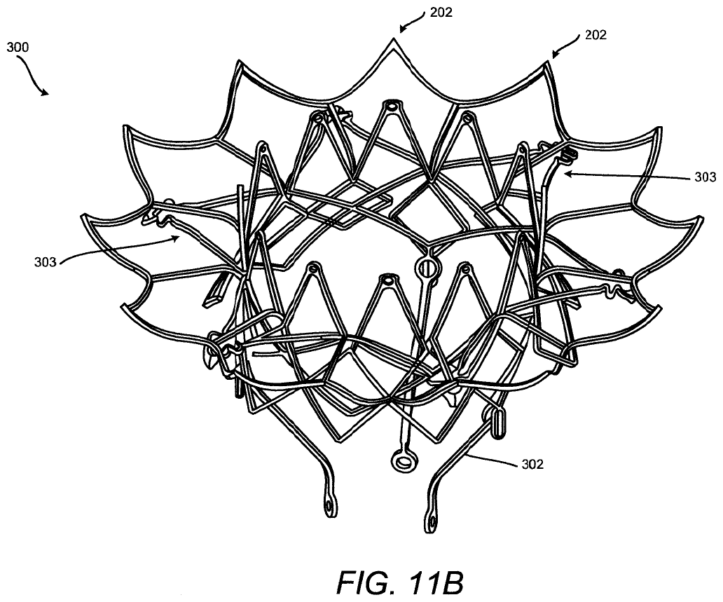

In one embodiment, prosthetic mitral valve system 300 is the shape set system created by docking prosthetic mitral valve 201 in its shape set configuration inside valve dock 301 in its shape set configuration as shown in FIGS. 11A-11B. When valve dock 301 is implanted at or adjacent to the native mitral valve annulus and prosthetic mitral valve 201 is docked inside this deployed valve dock, the resulting prosthetic mitral valve system will resiliently tend to the shape set prosthetic mitral valve system 300. As shown in these figures, the atrial clamp jaws 202 of prosthetic mitral valve 201 lie on the atrial side of the native mitral valve annulus and ventricular clamp jaws 303 line on the ventricular side of the native mitral valve annulus with the native mitral valve leaflets LF and native mitral valve annulus pinched or clamped between these two sets of jaws. Upon deployment, once the prosthetic mitral valve is anchored inside the valve dock, the atrial clamp jaws and ventricular clamp jaws serve to anchor the prosthetic mitral valve system at or adjacent to the native mitral valve annulus.

Although not shown, in the deployed state when prosthetic mitral valve 201 is anchored inside valve dock 301, sacrificial prosthetic leaflets are pushed against the inside surface of dock stent 306, thereby providing a cover on the outside surface of valve stent 206 of prosthetic mitral valve 201, which helps to alleviate paravalvular leakage (similar to prosthetic mitral valve system 200 shown in FIG. 7A). Upon deployment of the prosthetic mitral valve 202 at or adjacent to the native mitral valve annulus of a patient, prosthetic leaflets 207, shown in FIG. 7A, serve to regulate blood flow between the left atrium and left ventricle of the patient.

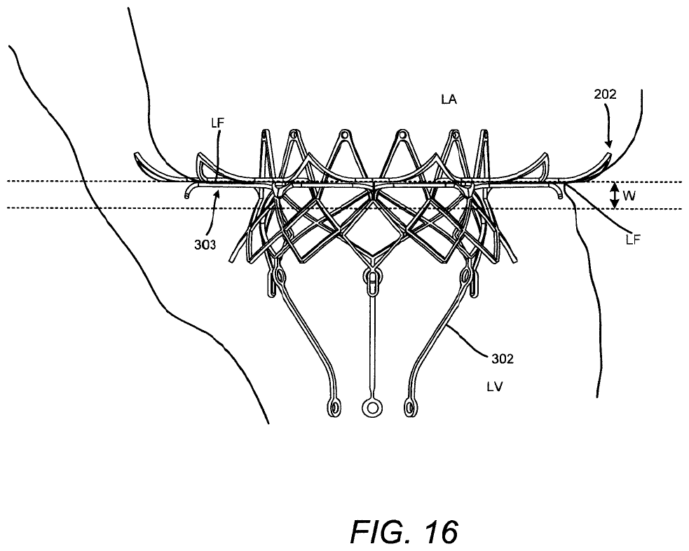

The relative location of atrial clamp jaws 202 and ventricular clamp jaws 303 in the shape set configuration of prosthetic mitral valve system 300 is such that when the valve dock is implanted at or adjacent to the native mitral valve annulus of a patient's heart, the two clamp jaws will act as a pair of forceps to clamp or pinch between them the native mitral valve leaflets and native mitral valve annulus as shown in FIGS. 15 and 16, and discussed hereinbelow. As shown in FIG. 11A, in the shape set configuration of prosthetic mitral valve system 300, and, therefore, in the shape set configurations of valve dock 301, and prosthetic mitral valve 201, a portion of the legs of ventricular clamp jaws 303 and a portion of the legs of atrial clamp jaws 202 are in close proximity to or touching each other. As will be discussed below, when the valve dock is deployed at or adjacent to the native mitral valve annulus, ventricular clamp jaws 303 will be positioned on the ventricular or downstream side of the native mitral valve annulus and the atrial clamp jaws 202 will be positioned on the atrial or upstream side of the native mitral valve annulus, with the native mitral valve leaflets and native mitral valve annulus sandwiched between them, which means that the two sets of clamp jaws have been separated apart so that they are no longer in close proximity to or touching each other and, therefore, have been elastically deformed from the their state as part of the shape set prosthetic mitral valve system. Accordingly, they will be resiliently biased to return to their shape state as part of the shape set prosthetic mitral valve system, where they are touching each other or are in closer proximity to each other compared to their deployed states, thereby pinching or clamping the native mitral valve leaflets and native mitral annulus between them.

The relative separation between atrial clamp jaws and the ventricular clamp jaws in the shape set configuration of prosthetic mitral valve system can be illustrated with reference to the embodiment shown in FIG. 11A, where line LL is perpendicular to the longitudinal axis of dock stent 306 and lies in the inflow plane of the dock stent. Longitudinal distance d from line LL to any point is defined as the distance measured from line LL to that point along a line that is perpendicular to line LL. If the point lies upstream of line LL (on the atrial side of line LL), d will be positive. For points that lie downstream of line LL (or on the ventricular side of line LL), distance d will be negative.

As can be seen with reference to FIG. 11A, the legs of the “U” shaped ventricular clamp jaws have a section that is substantially parallel to line LL and furthest from line LL. Therefore, d2, the longitudinal distance between any point on this section and line LL, is the maximum longitudinal distance between any point on ventricular clamp jaws 303 and line LL. Similarly, the legs of the “V” shaped atrial clamp jaws have a section that is substantially parallel to line LL and nearest to line LL. Therefore, d4, the longitudinal distance between any point on this section and line LL is the minimum longitudinal distance between any point on atrial clamp jaws 202 and line LL. In the illustrated embodiment, distances d4 and d2 are measured from line LL to sections of jaws 202 and 303, respectively, that are substantially parallel to line LL, and in general, they are defined as: d4 is the minimum longitudinal distance between atrial clamp jaw 202 and line LL and d2 is the maximum longitudinal distance between ventricular clamp jaws 303 and line LL.

If we define maximum relative separation d5 as being equal to d2−d4, in the embodiment shown in FIG. 11A i.e., in the shape set configuration of the prosthetic mitral valve system shown in this figure, d5 is approximately equal to 0 because ventricular clamp jaws 303 and atrial clamp jaws 202 are touching each other. If d5>0 in the shape set configuration of the prosthetic mitral valve system 300, some portion of ventricular clamp jaws 303 lies further upstream (or further in the proximal direction) from line LL than some portion of atrial clamp jaws 202 (not shown here but would be similar to the case for ventricular clamp jaws and inflow clamp jaws shown in FIGS. 1A and 2). In that case, because upon deployment the atrial clamp jaws are on the atrial side of the native mitral valve annulus and ventricular clamp jaws are on the ventricular side of the native mitral valve annulus, all of the atrial clamp jaws will be further upstream (or further in the atrial direction) than the ventricular clamp jaws and the native mitral valve leaflets and the native mitral valve annulus will be in between the two jaws, which means that the clamp jaws are elastically deformed from their shape set configuration. This means that the ventricular clamp jaws and atrial clamp jaws are resiliently biased with respect to each other and will have a tendency to move relative to each other to try to restore the shape set configuration of prosthetic mitral valve system 300. However, because deployment of valve dock 301 and docking of prosthetic mitral valve 201 inside the deployed valve dock 301 is done in such a way that the native mitral valve leaflets and the native mitral valve annulus are trapped between them, the resilient forces between the two sets of clamp jaws will result in the native mitral valve leaflets and native mitral valve annulus being pinched or clamped between them.

In the embodiments shown in FIGS. 11A, d2 is almost equal to d4 and, therefore, d5 is approximately equal to 0 and as discussed above this configuration of the ventricular and atrial clamp jaws will have the effect of these two sets of clamp jaws being resiliently biased with respect to each other when the valve dock and the prosthetic mitral valve are deployed, which will cause the native mitral valve leaflets and mitral valve annulus to be pinched or clamped between the ventricular and atrial clamp jaws. This effect will also obtain if d5>0. In that configuration, the ventricular clamp jaws will be further upstream from line LL compared to the atrial clamp jaws (not shown here but would be similar to the separation between inflow clamp jaws 102 and ventricular clamp jaws 103 shown in FIG. 1A). In other embodiments, d2 may even be less than d4 as long as the atrial clamp jaws and ventricular clamp jaws are sufficiently close to each other so that upon deployment of valve dock 301 and prosthetic mitral valve 202, native mitral valve leaflets LF are pressed between the clamp jaws 202 and 303. In general, given the anatomy of the native mitral valve, if d5>−4 mm (i.e., d5 measured in millimeters is greater than minus 4), ventricular clamp jaws 303 and atrial clamp jaws 202 upon deployment, i.e., when ventricular clamp jaws are downstream of the native annulus and atrial clamp jaws are upstream of the native annulus, will be sufficiently resiliently biased to pinch or clamp the native mitral valve leaflets and native mitral valve annulus between them.

In the embodiment shown in FIGS. 11A, sections of both the “U” shaped ventricular clamp jaws and the “V” shaped atrial clamp jaws are substantially parallel to line LL, these sections are substantially parallel to each other and, therefore, the legs of the “U” shaped ventricular clamp jaws have a section that is substantially parallel to a section of the legs of the “V” shaped atrial clamp jaws. In other embodiments, atrial clamp jaws 202 and ventricular clamp jaws 303 can be such that they do not have any sections that are parallel to each other or to line LL. In such embodiments, clamp jaws 202 and 303 would be resiliently biased so that the native mitral valve leaflets and the native mitral annulus is clamped between them if d5>−4 mm.

In treating a patient suffering from mitral valve regurgitation or other mitral valve insufficiency, valve dock 301 and prosthetic mitral valve 201 are deployed at or adjacent to the native mitral valve annulus of the patient such that prosthetic mitral valve 201 is deployed inside valve dock 301. FIGS. 12A-13 show the deployment of valve dock 301 at or adjacent to the native mitral valve annulus. As discussed before, after percutaneously accessing the femoral vein, a catheter having a needle or a guidewire is advanced into the right atrium RA through the inferior vena cava IVC. When the catheter is on the anterior side of the inter-atrial septum, the needle or guidewire is made to penetrate the inter-atrial septum. Following this, in the case where a needle is used instead of a guidewire, a guidewire is exchanged for the needle and the catheter is withdrawn. By placing a catheter over the guidewire access to the left atrium through the inter-atrial septum is maintained. In the illustrated embodiment, valve dock 301 (not shown) is crimped to a collapsed condition under sheath 316 on a catheter 315. Valve dock 301 is crimped so that ventricular clamp jaws 303 are parallel to the longitudinal axis with the clamp jaws bent in a proximal direction. Thus, the alignment of ventricular clamp jaws 301 with respect to dock stent 306 will be similar to the alignment of ventricular clamp jaws 103 with respect to dock stent 106 as illustrated in FIG. 4B, i.e., the angle between ventricular clamp jaws 301 and dock stent 306 is about 180 degrees. Furthermore, referring to FIG. 12A, in the crimped state, ventricular clamp jaws 303 will be nearer to the proximal end of catheter 315 than dock stent 306 is to the proximal end of catheter 315, and, therefore, dock stent 306 will be nearer to the distal end of catheter 315 compared to the distance of ventricular clamp jaws from the distal end.

Additionally, in this embodiment, catheter 315 includes a nose cone 319 attached to guidewire 320. In the crimped state of valve dock 301, anchor legs 302 are held inside nose cone 319.

During deployment, catheter 315 with the crimped valve dock under the catheter's sheath is advanced into the left ventricle just past the free edge of at least one of the native mitral valve leaflets LF. In that position, sheath 316 of catheter 315 is partially withdrawn proximally first releasing dock stent 306. Because nose cone 319 is attached to guidewire 320, withdrawing sheath 316 proximally does not withdraw nose cone 319, which stays in place holding the anchor legs 302. Because anchor legs 302 are secured by nose cone 319, deployment of valve dock 301 can be done in a secure, controlled manner minimizing the risk of embolization by migration of valve dock 301 into the left atrium LA.

Once proper positioning of dock stent clear of the native mitral valve leaflets has been verified, sheath 316 is further withdrawn proximally, keeping in place nose cone 319 holding anchor legs 302, releasing ventricular clamp jaws 303. On being released ventricular clamp jaws 303 will tend to assume their expanded state shape set configuration, i.e., they will extend radially outwards from the inflow end of dock stent 306 towards the inside wall of the left ventricle so that they are arranged around the circumference of dock stent 306 and aligned in a direction that is substantially perpendicular to the longitudinal axis of dock stent 306. With the ventricular clamp jaws thus released, catheter 315 is pulled proximally which pulls ventricular clamp jaws 303 towards the native mitral valve annulus which, in turn, push the native mitral valve leaflets LF up towards the native mitral valve annulus till the leaflets are crushed or pressed between the ventricular side of native mitral valve annulus AN and ventricular clamp jaws 303.

Once catheter 315 has been pulled back sufficiently to trap and tightly press the native mitral valve leaflets between ventricular clamp jaws 303 and the ventricular side of the native mitral valve annulus, two different approaches prior to deploying prosthetic mitral valve 201 can be used: in a first approach, catheter 315 is rotated prior to deploying prosthetic mitral valve 201, and in a second approach catheter 315 is not rotated prior to deploying prosthetic mitral valve 201.

In the first approach, after the leaflets have been trapped between ventricular clamp jaws 303 and the native mitral valve annulus, catheter 315 is rotated axially so that dock stent 306 and, therefore, ventricular clamp jaws 303 are rotated axially to entangle and/or capture the native chordae tendineae. This results in the native leaflets being more securely and uniformly spread out around ventricular clamp jaws 303 and beneath native mitral valve annulus AN. In one embodiment of this approach, a valve dock that has barbs on ventricular clamp jaws 303 is used similar to valve dock 101 shown in FIG. 3. When valve dock 301 that has ventricular clamp jaws with barbs is rotated by rotating catheter 315, one of more the barbs facilitate capture of the native chordae tendineae by preventing the chordae from slipping off the ventricular clamp jaws. Once the leaflets have been thus secured between ventricular clamp jaws 303 and native mitral valve annulus AN and chordae tendineae have been captured by ventricular clamp jaws 303, catheter 315 is withdrawn leaving nose cone 319 attached to guidewire 320 in place, still holding anchor legs 302.

In the second approach, once leaflets have been trapped between ventricular clamp jaws 303 and native mitral valve annulus AN, catheter 315 is not rotated but is simply withdrawn leaving nose cone 319 attached to guidewire 320 in place, still holding anchor legs 302.

At this point valve dock 301 is fully deployed at or adjacent to the native mitral valve annulus as shown in FIGS. 12B and 13. Ventricular clamp jaws 303 are seated on the ventricular side of the native mitral valve annulus, and leaflets LF are pushed back against the native mitral valve annulus by ventricular clamp jaws 303. Furthermore, valve dock 301 is held or anchored in place because anchor legs 302 of valve dock 301 are still held by nose cone 319.

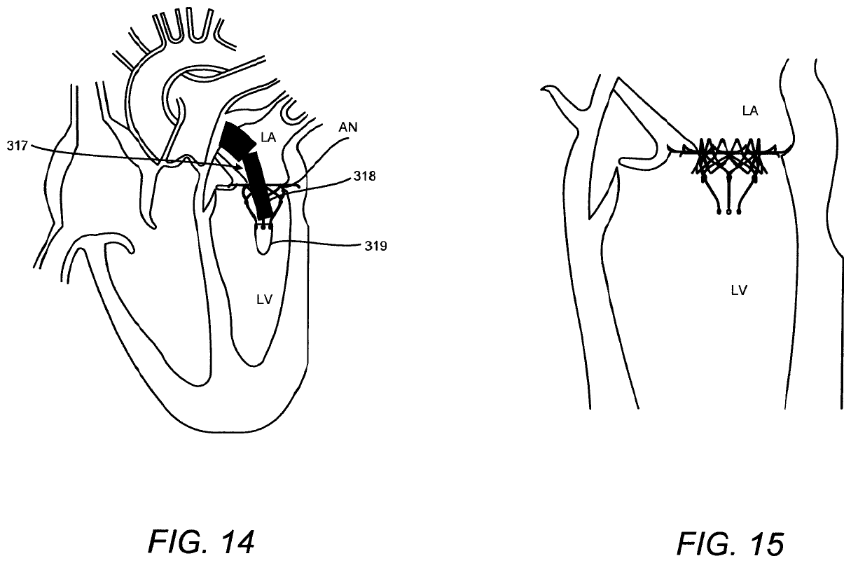

Following deployment and implantation of the valve dock, the over the wire catheter 315 for the dock valve is exchanged for another catheter that has crimped on it a prosthetic mitral valve. In one embodiment, prosthetic mitral valve 201 is delivered and deployed inside the implanted valve dock 301 at or adjacent to the native mitral valve annulus as shown in FIGS. 14-16.

In the illustrated embodiment, prosthetic mitral valve 201 (not shown) is crimped to a collapsed condition under sheath 318 on a catheter 317. Prosthetic mitral valve 201 is crimped so that atrial clamp jaws 202 are parallel to the longitudinal axis with the clamp jaws bent in a proximal direction. To illustrate, atrial clamp jaws 202 of prosthetic mitral valve 201 will be aligned with respect to the rest of prosthetic mitral valve 201 as shown in FIG. 6 as discussed hereinabove. Furthermore, in the crimped state, atrial clamp jaws, 202 referring to FIG. 14, will be nearer to the proximal end of catheter 317 than valve stent 206 is to the proximal end of catheter 317, and, therefore, valve stent 206 will be nearer to the distal end of catheter 317 compared to the distance of atrial clamp jaws from the distal end.

During deployment, catheter 317 with the crimped prosthetic mitral valve 201 (not shown) under the catheter's sheath is advanced just past the native mitral valve annulus into the left ventricle LV pushing back the sacrificial prosthetic leaflets. In that position, sheath 318 of catheter 317 is partially withdrawn proximally first releasing valve stent 206 inside the already deployed dock stent 306. Valve stent 206 is secured inside dock stent 306 by friction fit, interference fit, a lock and key type of fit, or other such approaches for securing a cylindrical object inside another cylindrical object.

Once valve stent 206 has been properly secured inside dock stent 306, sheath 318 is further withdrawn proximally, releasing atrial clamp jaws 202. On being released, atrial clamp jaws 202 will tend to assume their expanded state which means that they will bend down towards the distal end of catheter 317 until at least some part of atrial clamp jaws 202 press down on the atrial side of the native mitral valve annulus as shown in FIGS. 15 and 16. Because the ends of atrial clamp jaws 202 are bent up towards the proximal end of catheter 317 and away from the inside wall of the left atrium, atrial clamp jaws move towards their expanded state atraumatically without injuring the inside wall of the left atrium.

Once prosthetic mitral valve 201 has been deployed inside valve dock 301, nose cone 319 is pushed distally releasing anchor legs 302. Catheter 317 with nose cone 319 is then withdrawn from the body of the patient.

At this point atrial clamp jaws 202 are seated on the atrial side of the native mitral valve annulus as shown in FIGS. 15 and 16. Thus, leaflets LF are pinched or clamped between atrial clamp jaws 202 and ventricular clamp jaws 303 as shown in FIGS. 15 and 16. In the embodiment shown in these figures, leaflets LF are sandwiched between atrial clamp jaws 202 and ventricular clamp jaws 303 such that the leaflets are substantially confined or boxed in between sections of atrial clamp jaws 202 and ventricular clamp jaws 303 which are substantially perpendicular to the longitudinal axis of dock stent 306 or valve stent 206. As shown in FIG. 16, in some embodiments, leaflets LF are substantially pressed between atrial clamp jaws 202 and ventricular clamp jaws 303 and almost the entire mass of leaflets LF is closely confined in a narrow region around the native mitral valve annulus AN and between ventricular clamp jaws 303 and atrial clamp jaws 202. This region is defined as being bounded on the upstream side by the inflow plane of valve stent 206 and by a plane that is parallel to this plane on the downstream side where the longitudinal distance between the two planes and, therefore, the width of this region is W. In some embodiments, leaflets LF are substantially held in a region bounded by said two planes where W is less than 4 mm. In other embodiments, W is less than 3 mm. In yet other embodiments, W is less than 2 mm. As discussed above, this is to be distinguished from prior art prosthetic mitral valve designs where some part or all of the native mitral valve leaflets are allowed to remain in a direction that is more or less parallel to the longitudinal axis of the prior art prosthetic mitral valve or dock. Such designs tend to increase obstruction of the left ventricle outflow tract (LVOT), rendering such designs unacceptable for a vast number of patients suffering from mitral valve regurgitation or other insufficiency. Although the native mitral valve leaflets LF are pushed up to be held in the relatively narrow region bounded by atrial clamp jaws 202 and ventricular clamp jaws 303, leaflets LF continue to be attached to the papillary muscles through tendinae chordae.

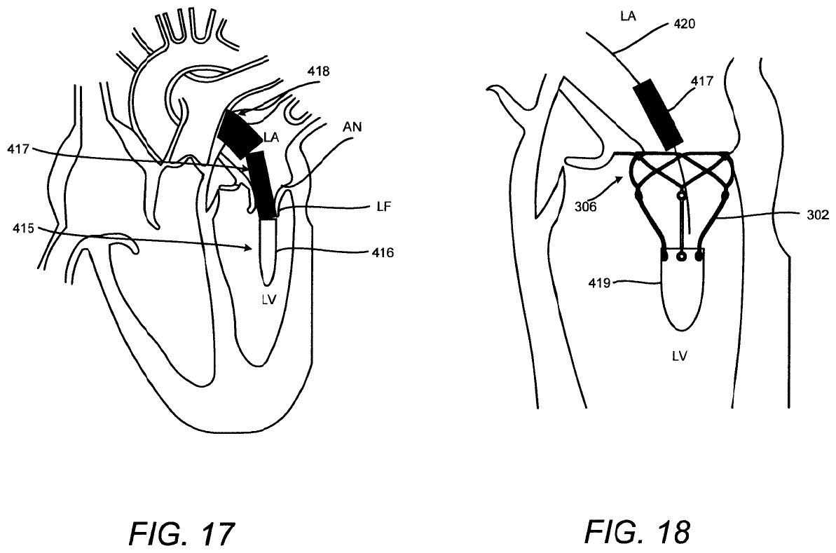

In the embodiment just discussed, prosthetic mitral valve system 300 comprising valve dock 301 and prosthetic mitral valve 201 is implanted at or adjacent to the native mitral valve annulus of a patient using two separate catheters: one for deploying valve dock 301 and another for deploying prosthetic mitral valve 201. In another embodiment, prosthetic mitral valve system 300 comprising valve dock 301 and prosthetic mitral valve 201 is implanted at or adjacent to the native mitral valve annulus of a patient using a single catheter: the same catheter is used for deploying valve dock 301 and for deploying prosthetic mitral valve 201 as shown in FIGS. 17-20.

As discussed before, after percutaneously accessing the femoral vein, a catheter having a needle or a guidewire is advanced into the right atrium RA through the inferior vena cava IVC. When the catheter is on the anterior side of the inter-atrial septum, the needle or guidewire is made to penetrate the inter-atrial septum. Following this, in the case where a needle is used instead of a guidewire, a guidewire is exchanged for the needle and the catheter is withdrawn. By placing a catheter over the guidewire access to the left atrium through the inter-atrial septum is maintained. In the illustrated embodiment, valve dock 301 (not shown) is crimped to a collapsed condition on the distal end 416 and prosthetic mitral valve is crimped on a proximal part 417 of sheath 418 on catheter 415. Valve dock 301 is crimped so that ventricular clamp jaws 303 are parallel to the longitudinal axis with the clamp jaws bent in a proximal direction. Thus, the alignment of ventricular clamp jaws 303 with respect to dock stent 306 will be similar to the alignment of ventricular clamp jaws 103 with respect to dock stent 106 as illustrated in FIG. 4B, i.e., the angle between ventricular clamp jaws 303 and dock stent 306 will about 180 degrees. Furthermore, referring to FIG. 17, in the crimped state, ventricular clamp jaws 303 will be nearer to the proximal end of catheter 415 than dock stent 306 is to the proximal end of catheter 415, and, therefore, dock stent 306 will be nearer to the distal end of catheter 415 compared to the distance of ventricular clamp jaws from the distal end.

As noted above, in the illustrated embodiment, prosthetic mitral valve 201 (not shown) is crimped to a collapsed condition on a proximal part 417 of sheath 418 on catheter 415. Prosthetic mitral valve 201 is crimped so that atrial clamp jaws 202 are parallel to the longitudinal axis with the clamp jaws bent in a proximal direction. To illustrate, atrial clamp jaws 202 of prosthetic mitral valve 201 will be aligned with respect to the valve stent 206 as shown in FIG. 6 as discussed hereinabove, i.e., the angle between atrial clamp jaws 202 and valve stent 206 is about 180 degrees. Furthermore, in the crimped state, atrial clamp jaws 202 referring to FIG. 17, will be nearer to the proximal end of catheter 415 than valve stent 206 is to the proximal end of catheter 415, and, therefore, valve stent 206 will be nearer to the distal end of catheter 415 compared to the distance of atrial clamp jaws from the distal end.

Additionally, in this embodiment, catheter 415 includes a nose cone 419 attached to guidewire 420. In the crimped state of valve dock 301, anchor legs 302 are held inside nose cone 419.

During deployment, catheter 415 with the crimped valve dock and crimped mitral valve under the catheter's sheath is advanced into the left ventricle LV just past the free end of at least one of the native mitral valve leaflets LF. In that position, sheath 418 of catheter 415 is partially withdrawn proximally first releasing dock stent 306. Because nose cone 419 is attached to guidewire lumen 420, withdrawing the sheath proximally does not withdraw nose cone 419, which stays in place holding the anchor legs 302. Because anchor legs 302 are secured by nose cone 419, deployment of valve dock 301 can be done in a secure, controlled manner minimizing the risk of embolization by migration of valve dock 301 into the left atrium LA.

Once proper positioning of dock stent clear of the native mitral valve leaflets annulus has been verified, sheath 418 of catheter 415 is further withdrawn proximally, keeping in place nose cone 419 holding anchor legs 302, releasing ventricular clamp jaws 303. On being released ventricular clamp jaws 303 will tend to assume their shape set configuration, i.e., they will extend radially outwards from the inflow end of dock stent 306 towards the inside wall of the left ventricle so that they are arranged around the circumference of dock stent 306 and extend substantially radially outwards from dock stent 306 as shown in FIG. 18. With the ventricular clamp jaws thus released, catheter 415 is pulled proximally which pulls the ventricular clamp jaws 303 towards the native mitral valve annulus which, in turn, push the native mitral valve leaflets towards the native mitral valve annulus until the leaflets are crushed or pressed between the ventricular side of the native mitral valve annulus and ventricular clamp jaws 303.

Once catheter 415 has been pulled back sufficiently to trap and press the native mitral valve leaflets between the ventricular side of the native mitral valve annulus and ventricular clamp jaws 303, two different approaches prior to deploying prosthetic mitral valve 201 can be used: in a first approach, catheter 415 is rotated prior to deploying prosthetic mitral valve 201, and in a second approach catheter 415 is not rotated prior to deploying prosthetic mitral valve 201.

In the first approach, once the native mitral valve leaflets have been trapped by ventricular clamp jaws 303 and the native mitral valve annulus, catheter 415 is rotated axially so that dock stent 306 and, therefore, ventricular clamp jaws 303 are rotated axially to entangle and/or capture the native chordae tendineae. This results in the native leaflets being more securely and uniformly spread out around ventricular clamp jaws 303 and beneath native mitral valve annulus AN. In one embodiment of this approach, a valve dock that has barbs on ventricular clamp jaws 303 is used similar to valve dock 101 shown in FIG. 3A. When valve dock 301 that has ventricular clamp jaws with barbs is rotated by rotating catheter 415, one or more of the barbs facilitate capture of the native chordae tendineae by preventing the chordae from slipping off the ventricular clamp jaws. Once the native mitral valve leaflets have been thus secured between ventricular clamp jaws and native mitral valve annulus and chordae tendineae have been captured by ventricular clamp jaws 303, with nose cone 419 attached to guidewire 420 in place, still holding anchor legs 302, prosthetic mitral valve 201 is deployed as discussed below.

In the second approach, once the native mitral valve leaflets have been trapped between ventricular clamp jaws 303 and native mitral valve annulus, catheter 415 is not rotated and prosthetic mitral valve 201 is deployed as discussed below.

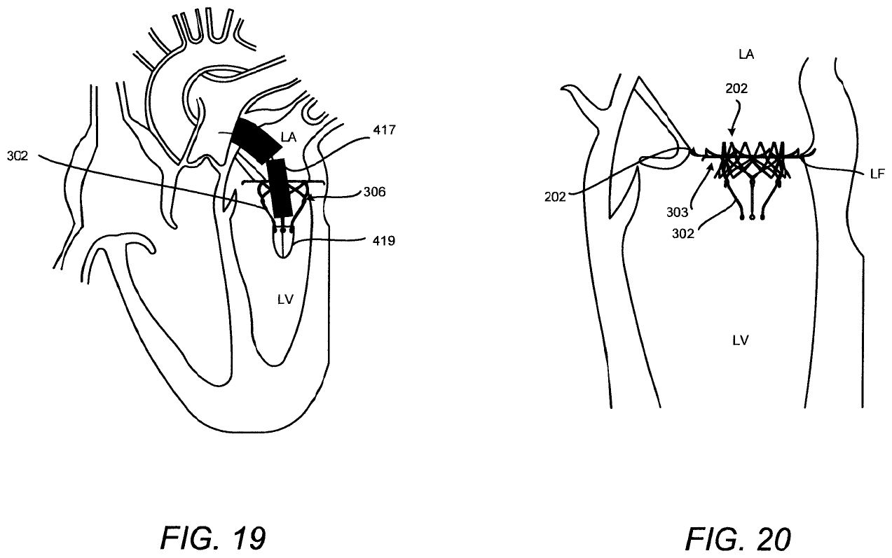

At this point valve dock 301 is fully deployed at or adjacent to the native mitral valve annulus as shown in FIGS. 18 and 19. Ventricular clamp jaws 303 are seated on the ventricular side of the native mitral valve annulus, and leaflets LF are pushed back against the native mitral valve annulus by ventricular clamp jaws 303. Furthermore, valve dock 301 is held or anchored in place because anchor legs 302 of valve dock 301 are still held by nose cone 419.

To deploy mitral valve 201, catheter 415 with the crimped prosthetic mitral valve 201 (not shown) under the catheter's sheath is advanced just past the native mitral valve annulus into the left ventricle LV pushing back the sacrificial prosthetic leaflets of valve dock 301. In that position, sheath 418 of catheter 415 is partially withdrawn proximally first releasing valve stent 206 inside the already deployed dock stent 306. Valve stent 206 is secured inside dock stent 306 by friction fit, interference fit, a lock and key type of fit, or other such approaches for securing a cylindrical object inside another cylindrical object.

Once valve stent 206 has been properly secured inside dock stent 306, sheath 418 is further withdrawn proximally, releasing atrial clamp jaws 202. On being released, atrial clamp jaws 202 will tend to assume their expanded shape set configuration which means that they will bend down towards the distal end of catheter 415 until at least some part of atrial clamp jaws 202 presses down on the atrial side of the native mitral valve annulus. Because the ends of atrial clamp jaws 202 are bent up towards the proximal end of catheter 415 and away from the inside wall of the left atrium, atrial clamp jaws move towards their expanded state atraumatically without injuring the inside wall of the left atrium.

Once prosthetic mitral valve 201 has been deployed inside valve dock 301, nose cone 419 is pushed distally releasing anchor legs 302. Catheter 415 with nose cone 419 is then withdrawn from the body of the patient.

At this point atrial clamp jaws 202 are seated on the atrial side of the native mitral valve annulus as shown in FIGS. 16 (which is the deployed configuration for the valve dock and prosthetic mitral valve for both the single catheter delivery embodiment and two catheter delivery embodiment) and 20. Thus, native mitral valve leaflets LF are pinched or clamped between atrial clamp jaws 202 and ventricular clamp jaws 303 as shown in FIGS. 16 and 20. In the embodiment shown in these figure, native mitral valve leaflets LF are sandwiched between atrial clamp jaws 202 and ventricular clamp jaws 303 such that the leaflets are substantially confined or boxed in between atrial clamp jaws 202 and ventricular clamp jaws 303 which are substantially perpendicular to the longitudinal axis of dock stent 306 or valve stent 206. As shown in FIG. 16, in some embodiments, native mitral valve leaflets LF are substantially pressed between atrial clamp jaws 202 and ventricular clamp jaws 303 and almost the entire mass of leaflets LF is closely confined in a narrow region around the native mitral valve annulus AN and between ventricular clamp jaws 303 and atrial clamp jaws 202. This region is defined as being bounded on the upstream side by the inflow plane of valve stent 206 and by a plane that is parallel to this plane on the downstream side where the longitudinal distance between the two planes and, therefore, the width of this region is W. In some embodiments, leaflets LF are substantially held in a region bounded by said two planes where W is less than 4 mm. In other embodiments, W is less than 3 mm. In yet other embodiments, W is less than 2 mm. As discussed above, this is to be distinguished from prior art prosthetic mitral valve designs where some part or all of the native mitral valve leaflets are allowed to remain in a direction that is more or less parallel to the longitudinal axis of the prior art prosthetic mitral valve or dock. Such designs tend to increase obstruction of the left ventricle outflow tract (LVOT), rendering such designs unacceptable for a vast number of patients suffering from mitral valve regurgitation or other insufficiency. Although the native mitral valve leaflets LF are pushed up to be held in the relatively narrow region bounded by atrial clamp jaws 202 and ventricular clamp jaws 303, leaflets LF continue to be attached to the papillary muscles through tendinae chordae.

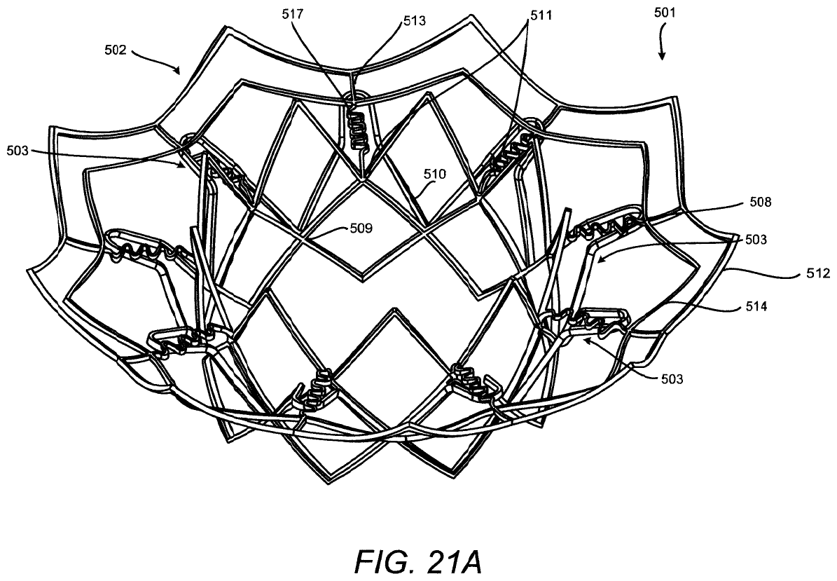

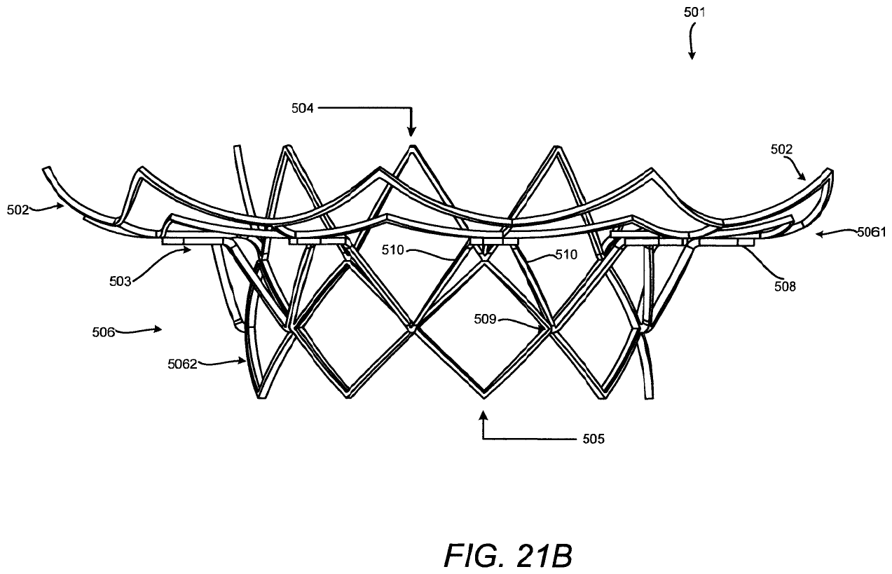

In another embodiment shown in FIG. 21A and 21B, prosthetic mitral valve 501 can be used to replace a diseased or malfunctioning native mitral valve of a patient. Prosthetic mitral valve 501 can be used without a valve dock. As shown in FIG. 21A, the prosthetic mitral valve includes a valve stent 506 which has an inflow end 504 which is the end that would be nearest to the left atrium of the patient's heart (hence the end through which blood would enter the prosthetic mitral valve from the left atrium) post implantation of the prosthetic mitral valve and an outflow end 505 which would be the end furthest from the left atrium of the patient's heart (hence the end out which blood would exit the prosthetic mitral valve into the left ventricle) post implantation of the prosthetic mitral valve, wherein the valve stent comprises an atrial valve stent 5061 and a ventricular valve stent 5062. Throughout this specification, “inflow end” of a device is that end of the device into which blood flows from the atrium into the device when the device has been implanted in a patient's heart, and “outflow end” of a device is that end of the device out which blood flows from the device into the ventricle when the device has been implanted in a patient's heart. Valve stent 506 may be made from a single tube (or wire mesh tube) in which part of the tube comprises atrial valve stent 5061 and part of the tube comprises ventricular valve stent 5062. Alternatively, valve stent 506 may be made from two separate tubes (or wire mesh tubes), one forming atrial valve stent 5061 and the other forming ventricular valve stent 5062, which are joined together to form valve stent 506.

As shown in FIG. 21A, connected to atrial valve stent 5061 are one or more atrial clamp jaws 502, and connected to ventricular valve stent 5062 are one or more ventricular clamp jaws 503. In the embodiment shown in FIG. 21B, the vertices of adjacent atrial clamp jaws 502 are 40 degrees apart from each other and equidistant from each other and vertices of adjacent ventricular clamp jaws 503 are 40 degrees apart from each other and equidistant from each other. Because the clamp jaws are arranged in a circle, the total number of clamp jaws is equal to 360 divided by the number of degrees by which vertices of adjacent clamp jaws are separated from each other. Thus, in this embodiment, there are nine (360/40) atrial clamp jaws 502 and nine ventricular clamp jaws 503, arranged equidistant from each other around inflow end 504. In various embodiments, atrial clamp jaws 502 and/or ventricular clamp jaws 503 can be evenly spaced from each other, where vertices of adjacent clamp jaws are from 20-180 degrees apart. In other embodiments, atrial clamp jaws 502 and/or ventricular clamp jaws 503 can be unevenly spaced from each other. Thus, for example, one set of adjacent clamp jaws can be 20 degrees apart whereas another adjacent set can be 60 degrees apart.

In the embodiment of the prosthetic mitral valve shown in FIGS. 21A and 21B, one or more ventricular clamp jaws 503 comprises a U-shaped valley end 508 and legs 510 bent in the direction of ventricular valve stent 5062 and connected to it at 509.

In the embodiment shown in FIG. 21A and B, atrial clamp jaws 502 comprise two nested V-shaped valley ends, 512 and 514 that are connected to legs 513 which are connected to atrial valve stent 5061 at 511. The V-shaped valley ends, 512 and 514 of atrial clamp jaws 502 are curved up and away from the inflow end of valve stent 506, such that post implantation V-shaped valley end 512 will point away from the inside wall of the left atrium, making them atraumatic.

Atrial clamp jaws 502 and/or ventricular clamp jaws 503 may also be made atraumatic by wrapping the clamp jaws with tissue, such as bovine or porcine pericardium tissue, or by other materials such as polytetrafluoroethylene (PTFE).

In the embodiment shown in FIGS. 21A and 21B, legs 513 of atrial clamp jaws 502 that connect to atrial valve stent 5061 include a strain relieving feature 517. The strain relieving feature comprises a wiggle or serpentine strut, which allows the prosthetic mitral valve to be flexible along the radial direction while retaining rigidity along the longitudinal axis of the valve stent. Other benefits of this feature include, inter alia, the following: (i) it allows the prosthetic mitral valve to be crimped to its delivery state within a delivery sheath with a lower radial force, (ii) it allows the outflow end to be more fully expanded while the inflow end is still crimped in the delivery sheath and (iii) it decouples the outflow end deformation from the inflow end deformation.

The atrial clamp jaws 502 and ventricular clamp jaws 503 are resiliently biased with respect to each other such that they act cooperatively to form a spring clamp that can tightly grip any material, such as tissue, trapped between the two sets of jaws. FIGS. 21A and 21B show prosthetic mitral valve 501 in its deployed state. In this state, atrial clamp jaws 502 will be on the atrial side of the native mitral valve annulus and the ventricular clamp jaws will be on the ventricle side of the native mitral valve annulus. Thus, as can be seen in FIGS. 21A and 21B, ventricular clamp jaws 503 are more proximal to outlet end 505 compared to atrial clamp jaws 502 which are more distal to outlet end 505. Although not shown, in the deployed state of prosthetic mitral valve 501, native mitral valve leaflets and/or native mitral valve annulus will be clamped between atrial and ventricular clamp jaws, 502 and 503, respectively.

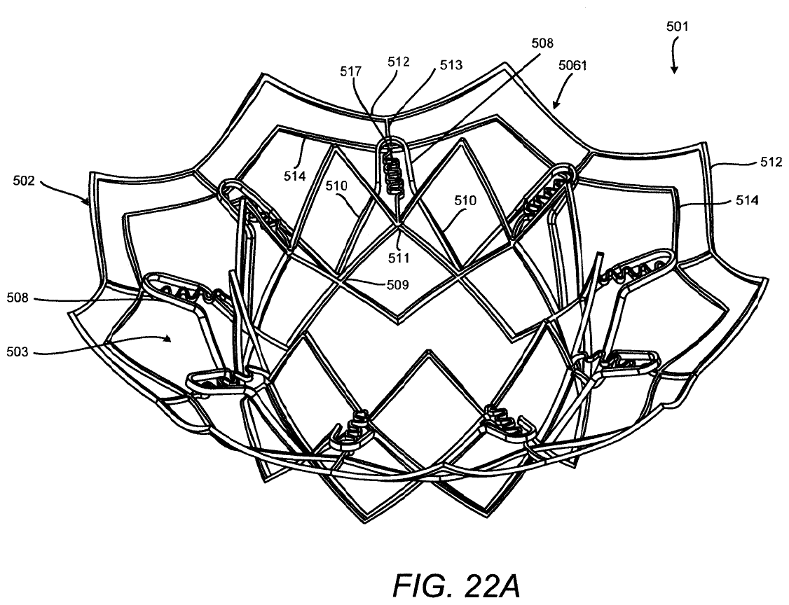

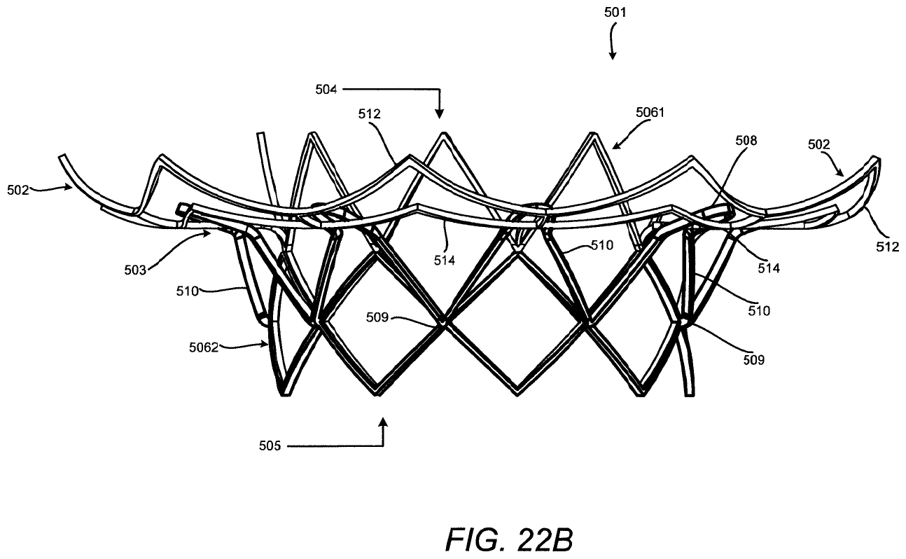

The atrial clamp jaws 502 and ventricular clamp jaws 503 are resiliently biased with respect to each other such that they act cooperatively to form a spring clamp that can tightly grip any material, such as valve leaflets, between the two sets of jaws. In one embodiment, this is achieved by shapesetting prosthetic mitral valve 501 such at least a part of ventricular clamp jaws 503 would be more distal to outflow end 505 in the shape set configuration of prosthetic mitral valve 501 than in the deployed configuration of prosthetic mitral valve 501 (shown in FIGS. 21A and 21B). This can be seen in FIGS. 22A and 22B, which show prosthetic mitral valve 501 in said shape set configuration, which is the configuration that would be achieved if all the components of prosthetic mitral valve were able to reach their shape set configuration. As can be seen, in such a configuration, part of U-shaped valley end 508 of ventricular clamp jaws 503 overlaps part of V-shaped valley end 514 and leg 513 of atrial clamp jaws 502 and at the points of overlap U-shaped valley end 508 is more distal to outflow end 505 compared to V-shaped valley end 514 and leg 513 which are more proximal to outflow end 505. Because in the deployed configuration (shown in FIGS. 21A and 21B), at the points of overlap U-shaped valley end 508 is more proximal to outflow end 505 compared to V-shaped valley end 514 and leg 513 which are more distal to outflow end 505, at least a part of U-Shaped valley end 508 is more distal to outflow end 505 in the shape set configuration of prosthetic mitral valve 501 than in the deployed configuration of prosthetic mitral valve 501.

In some embodiments this is achieved by using two separate stents for the ventricular clamp jaws and atrial clamp jaws. Thus, atrial valve stent 5061 and ventricular valve stent 5062 are two separate stents. In such a case, atrial valve stent has an outside diameter that is less than the inside diameter of the ventricular valve stent. The atrial valve stent is then nested inside the ventricular valve stent and the two stents are joined together, for example, by suturing them together or by welding them together. This will create the prosthetic mitral valve 501 embodiment illustrated in FIGS. 21A and B, which would be the deployed prosthetic mitral valve configuration. Thus, valve stent 506 is a combination of atrial valve stent 5061 and ventricular valve stent 5062. We can also define a shape set configuration of prosthetic mitral valve 501 which is illustrated in FIGS. 22A and 22B. In this configuration, ventricular valve stent 5062 in its shape-set configuration is superimposed on atrial valve stent 5061 in its shape set configuration—this is the hypothetical configuration that would be achieved if atrial valve stent 5061 and ventricular valve stent 5062 could reach their shape set configurations when the two valve stents are joined together. Comparing FIGS. 21 and 22, it can be seen that where U-shaped valley end 508 overlaps with leg 513, U-shaped valley end 508 is more proximal to outflow end 505 compared to leg 513 in FIG. 21, whereas in FIG. 22 U-shaped valley end 508 is more distal to outflow end 505 compared to leg 513. This is so because in the deployed configuration of prosthetic mitral valve 501, although U-shaped valley end 508 will tend towards the more distal (with respect to outflow end 505) shape set configuration of FIG. 22, it is prevented from reaching that state by the presence of leg 513. This results in atrial clamp jaws 502 and ventricular clamp jaws 503 being resiliently biased with respect to each other. They will resiliently cooperate with each other to form a spring like clamp.

When prosthetic mitral valve 501 is deployed at the site of a native valve, it will tend to assume its shape set configuration, which means that part of U-shaped valley end 508 will tend towards a more distal position with respect to outflow end 505 compared to part of V-shaped valley end 514 and leg 513. However, the presence of native mitral valve leaflets LF, native mitral valve annulus AN and atrial clamp jaws 502 will restrain U-shaped valley end 508 of ventricular clamp jaws 503 from moving distally which means that in the deployed state of prosthetic mitral valve 501 atrial and ventricular clamp jaws will be elastically deformed from their shape set configuration, and, therefore, they will be resiliently biased to return to their shape-set state, thereby pinching or clamping the native mitral valve leaflets and native mitral annulus between them.

It is not necessary that in the shape set configuration, at the points where atrial and ventricular clamp jaws, 502 and 503, respectively, overlap with each other, the overlapping part of ventricular clamp jaws 503 be more distal with respect to outflow end 505 compared to the overlapping part of atrial clamp jaws 502. The jaws can be cooperatively resilient even if in the shape set configuration at overlap points ventricular clamp jaws 503 are more proximal with respect to outflow end 505 compared to atrial clamp jaws 502. In such a case, at overlap points, the separation between atrial clamp jaws and ventricular clamp jaws, 502 and 503, respectively, should be less than or equal to four millimeters (4 mm).

The radial lengths of the clamp jaws 502 and 503 should be sufficient to provide a stable and secure anchor for implanting the prosthetic mitral valve at the site of the native mitral valve that is being replaced. In practice this means that in some embodiments, the atrial clamp jaws 502 will be long enough to almost touch the inside wall of the left atrium and ventricular clamp jaws 503 will be long enough to almost touch the inside wall of the left ventricle. In other embodiments, the atrial clamp jaws 502 will have a length that extends in the radial direction to a distance that is 50% of the distance from the valve stent to the inside wall of the left atrium and ventricular clamp jaws 503 will have a length that extends to a distance that is 50% of the distance from the valve stent to the inside wall of the left ventricle. Other embodiments that have clamp jaws of intermediate lengths are also contemplated. The atraumatic design of the ventricular clamp jaws, such as of the clamp jaws 503 shown in FIG. 21A where the ends of the clamp jaws are curved away from the surface of the native leaflets and the wall of the left ventricle, allows the prosthetic mitral valve to be deployed without injuring the native leaflets or the wall of the left ventricle.

To regulate blood flow, prosthetic mitral valve 501 also includes prosthetic leaflets (not shown but they would be similar to prosthetic leaflets 207 of prosthetic mitral valve 201 as shown in FIGS. 5A, 5B and 5C). Thus, prosthetic mitral valve 501 comprises a plurality of prosthetic leaflets supported by and within the valve stent 506. The plurality of prosthetic leaflets and concomitant structure serve to regulate blood flow through the prosthetic mitral valve. The prosthetic leaflets can comprise materials, such as bovine or porcine pericardial tissue or synthetic materials. The prosthetic leaflets can be mounted to the valve stent 506 using well-known techniques and mechanisms. For example, the prosthetic leaflets can be sutured to valve stent 506 in a tricuspid arrangement (similarly to that for leaflets of prosthetic mitral valve 201 as shown in FIG. 4C). In some embodiments, prosthetic leaflets are mounted onto atrial valve stent 5061, whereas in other embodiments, prosthetic leaflets are mounted onto ventricular valve stent 5062.

The alignment of the components of the prosthetic mitral valve when it is crimped onto a catheter under a sheath for deployment would be such that the valley ends 512 and 508 of atrial clamp jaws 502 and ventricular clamp jaws 503, respectively, are located proximally on the catheter whereas outflow end 505 of valve stent 506 is located distally.

Each of the valve stents 506, 5061 and 5062 can be a tubular structure made of, for example, a wire mesh and can be radially collapsible and expandable between a radially expanded state and a radially compressed state for delivery and implantation at or adjacent to a native mitral valve annulus. The wire mesh can include metal wires or struts arranged in a lattice pattern. Said valve stents can be made of a shape-memory material, for example Nitinol, which makes the stents self-expandable from a radially compressed state to an expanded state. Alternatively, said valve stents can be plastically expandable from a radially compressed state to an expanded state using, for example, an inflatable balloon. Exemplary materials for such balloon expandable stents are stainless steel, chromium alloys, and/or other materials known to persons of ordinary skill in the art. As noted earlier, valve stent 506 can comprise a single tube or it can be made from two separate tubes, one for atrial valve stent 5061 and one for ventricular valve stent 5062, which are then combined to form valve stent 506. The prosthetic mitral valve leaflets may be attached to either the atrial valve stent or the ventricular valve stent. Both the atrial valve stent 5061 and the ventricular valve stent 5062 may be made from shape memory materials (self-expanding) or either of these two valve stents may be made from plastically deformable balloon expandable materials.

In another embodiment of prosthetic mitral valve 501, ventricular clamp jaws 503 are further provided with one or more barbs (not shown here but would be similar to ventricular clamp jaws 103 provided with one or more barbs 114 as shown in FIG. 3). As will be discussed hereinbelow, the barbs facilitate capture of the native chordae tendineae during one approach to deployment of prosthetic mitral valve 501 at or adjacent to the native mitral valve annulus.

Prosthetic mitral valve 501 can have a delivery configuration (not shown), a deployed configuration (FIGS. 21A-B), and a shape set configuration (FIGS. 22A and 22B). In the delivery configuration, prosthetic mitral valve 501 has a low profile suitable for delivery through small-diameter catheters positioned in the heart via the trans-septal, retrograde, or trans-apical approaches described hereinabove. In some embodiments, the delivery configuration of prosthetic mitral valve 501 will preferably have an outer diameter no larger than about 6-14 mm for trans-septal approaches, about 6-14 mm for retrograde approaches, or about 6-16 mm for trans-apical approaches to the native mitral valve. As used herein, “expanded configuration” refers to the configuration of the device (i) when allowed to freely expand to an unrestrained size without the presence of constraining or distorting forces when the valve stent is self-expanding, and (ii) when the device is expanded to its larger size by applying pressure on the inside of the valve stent via, for example, an inflatable balloon. “Deployed configuration,” as used herein, refers to the device once expanded at the native valve site, engaging components of the native anatomy such as native mitral valve leaflets for implantation at or adjacent to the native mitral valve annulus.

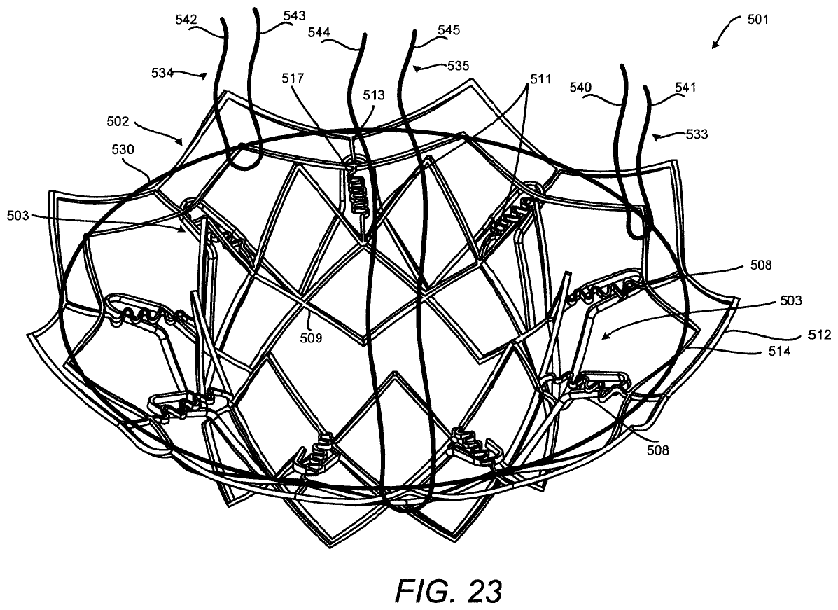

In treating a patient suffering from mitral valve regurgitation or other mitral valve insufficiency, prosthetic mitral valve 501 would be deployed at or adjacent to the native mitral valve annulus of the patient. The placement of the valve can be controlled using removeable suture loops, a rod or a wire which can be connected to the circumferential suture at the distal end and manipulated by the operator at the proximal end. In one embodiment, circumferential suture 530 is attached to prosthetic mitral valve 501, for example, as shown in FIG. 23. In the illustrated embodiment, prosthetic mitral valve 501 has circumferential suture 530 is threaded through atrial clamp jaws 502. In various embodiments the circumferential suture can be attached to a valve by threading it through one or more atrial clamp jaws, or by threading it through fabric covering the atrial clamp jaws. Once a circumferential suture has been attached to a prosthetic mitral valve, one or more removeable suture loops are looped through the circumferential suture. These removeable suture loops are shown in the illustrated embodiment in FIG. 23 as removeable suture loop 533, with free ends 540 and 541, removeable suture loop 534 with free ends 542 and 543, and removeable suture loop 535 with free ends 544 and 545. To load the prosthetic mitral valve onto a delivery catheter, first a small lumen catheter which has a snare at its distal end is inserted through the proximal end of the handle of the delivery catheter and fed through it until it exits the distal end of the delivery catheter. The free ends of the removeable suture loops are then tied to the snare, and the small lumen catheter is pulled out of the proximal end of the handle of the delivery catheter along with the free ends of the removeable suture loops tied to the snare. The prosthetic mitral valve is then crimped and loaded under the sheath of the delivery catheter. In one embodiment, the free ends of the removeable suture loops can be tied to a suture anchor (not shown). After the prosthetic mitral valve has been deployed at or near the native annulus, for example, the native mitral valve annulus, the operator can pull on the free ends of the removeable suture loops (which are connected to the prosthetic mitral valve through the circumferential suture) to adjust and/or fine tune prosthetic mitral valve deployment. Once the operator is satisfied with the deployment of the prosthetic mitral valve, the removeable suture loops can be pulled out of the proximal end of the handle of the delivery catheter by simply pulling one free end of each removeable suture loop. In some other embodiments, instead of connecting removeable suture loops to the circumferential suture, a wire or a rod is connected to the circumferential suture. In some embodiments, the rod or wire has a hook at its distal end which can be connected to the circumferential suture by twisting the rod or wire in one direction and which can be decoupled from the circumferential suture by twisting in the other direction.

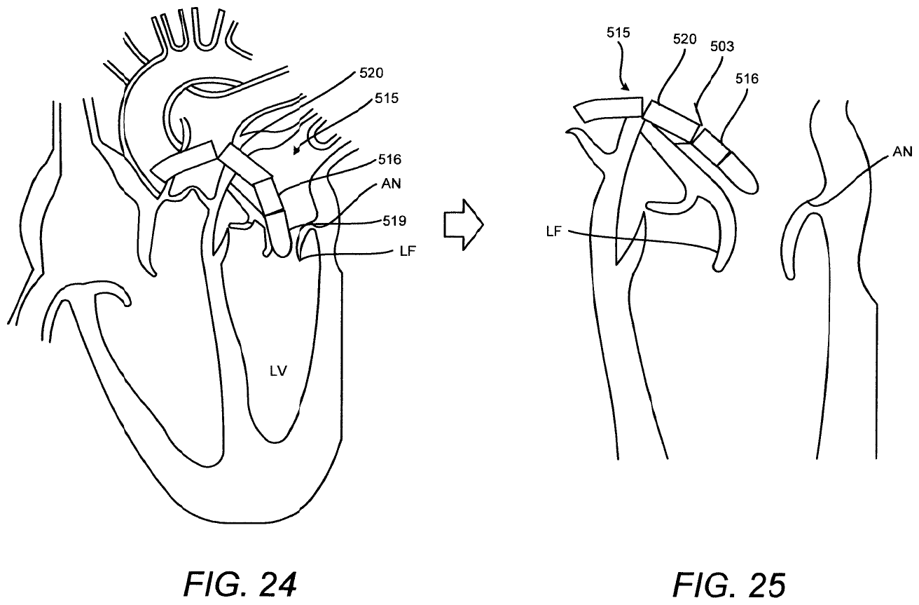

FIGS. 24-29 show the deployment of prosthetic mitral valve 501 at or adjacent to the native mitral valve annulus AN in such a way as to clamp leaflets LF and/or the annulus AN between atrial clamp jaws 502 and ventricular clamp jaws 503. As discussed before, after percutaneously accessing the femoral vein, a catheter having a needle or a guidewire is advanced into the right atrium RA through the inferior vena cava IVC. When the catheter is on the anterior side of the inter-atrial septum, the needle or guidewire is made to penetrate the inter-atrial septum. Following this, in the case where a needle is used instead of a guidewire, a guidewire is exchanged for the needle and the catheter is withdrawn. By placing a catheter over the guidewire access to the left atrium through the inter-atrial septum is maintained.

In the illustrated embodiment, prosthetic mitral valve 501 (not shown) is crimped to a collapsed condition under the sheath of delivery catheter 515, which comprises a proximal capsule 520, a distal capsule 516 and a nose cone 519. Prosthetic mitral valve 501 (not shown) is crimped so that the atrial clamp jaws and ventricular clamp jaws are parallel to the longitudinal axis with the clamp jaws bent in a proximal direction and crimped under proximal capsule 520, and the outflow end of valve stent 506 (not shown) is crimped under distal capsule 516. Further, though not shown here, atrial clamp jaws and/or ventricular clamp jaws are covered with fabric, and a circumferential suture is attached to either the fabric covering or threaded through the atrial clamp jaws, with one or more removeable suture loops connected to the circumferential suture. The removeable suture loops extend through the delivery catheter body and emerge out of the proximate end of the delivery catheter handle, so that they are accessible to the operator for adjusting the positioning of the prosthetic mitral valve if necessary.

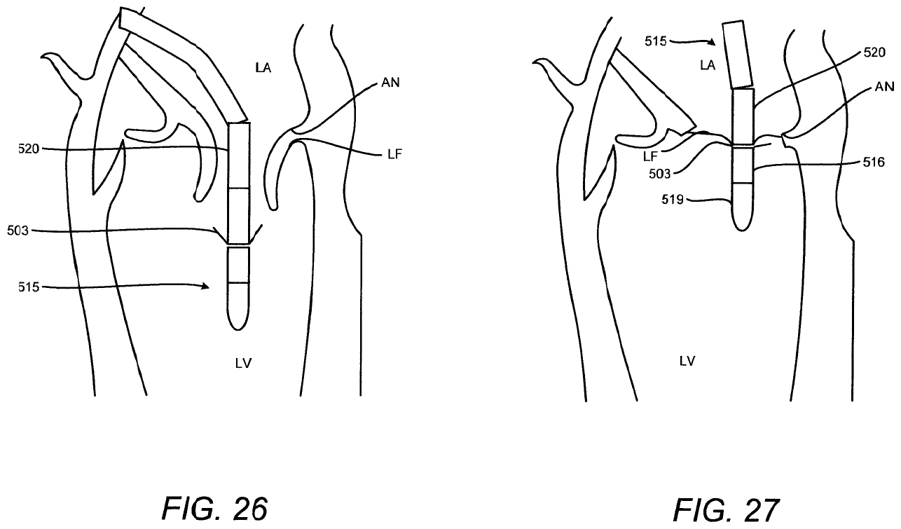

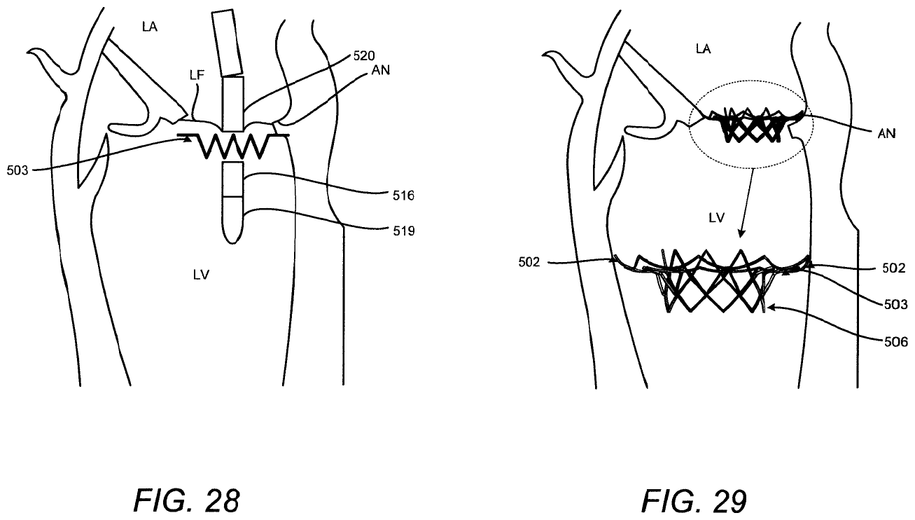

During deployment, catheter 515 with the crimped prosthetic mitral valve under the catheter's sheath is advanced into the left atrium LA and then proximal capsule 520 is pulled proximally to release ventricular clamp jaws 503 as shown in FIG. 25. On being released ventricular clamp jaws 503 will tend to assume their expanded shape set configuration, i.e., they will extend radially outwards from the sheath of delivery catheter 515. With ventricular clamp jaws 503 thus deployed, delivery catheter 515 is pushed distally until ventricular clamp jaws are below the distal edges of both native mitral valve leaflets LF as shown in FIG. 26. Once it has been verified that ventricular clamp jaws 503 have been pushed distally far enough into the left ventricle LV to be clear of the distal edges of native mitral valve leaflets LF, delivery catheter 515 is pulled proximally until ventricular clamp jaws 503 are approximately touching the ventricular side of the native mitral valve annulus AN, whereby native mitral valve leaflets LF are pushed back towards the ventricular side of the native mitral valve annulus AN as shown in FIG. 27. Distal capsule 516 is then pushed distally to release the outflow end of valve stent 506 as shown in FIG. 28. In some embodiments, the heart is subjected to rapid pacing to facilitate capture of the native mitral valve leaflets. This can be done prior to pulling delivery catheter 515 proximally until the ventricular clamp jaws are abutting the ventricular side of the native mitral valve annulus, or prior to deployment of the outflow end of valve stent 506. Once deployment of the outflow end of valve stent 506 and ventricular clamp jaws 503 on the ventricular side of native mitral valve annulus is confirmed, proximal capsule 520 is pulled such that the inflow end of valve stent 506 and atrial clamp jaws 502 are released on the atrial side of native mitral valve annulus AN as shown in FIG. 29. Proper positioning of prosthetic mitral valve 501 is then checked and if the position needs to be adjusted, operator pulls/tugs one or more of the removeable suture loops to adjust the valve. Once proper position is achieved, the removeable suture loops and delivery catheter 515 are pulled out of the body of the patient.

In another embodiment, after deployment of the outflow end of valve stent 506 and ventricular clamp jaws 503 on the ventricular side of native mitral valve annulus, delivery catheter 515 is rotated axially so that ventricular clamp jaws 503 are rotated axially to entangle and/or capture the native chordae tendineae. This results in the native leaflets being more securely and uniformly spread around ventricular clamp jaws 503 and underneath the native mitral valve annulus. In one embodiment of this approach, a prosthetic mitral valve with barbs on its ventricular clamp jaws is used (not shown here but would be similar to barbs 114 shown on valve dock 101 as shown in FIG. 3) instead of prosthetic mitral valve 501 shown in FIGS. 21A and B. When ventricular clamp jaws 503 are rotated by rotating delivery catheter 515, one of more barbs will facilitate capture of the native chordae tendineae by preventing the chordae from slipping off ventricular clamp jaws 503. Proximal capsule 520 is then pulled proximally such that the inflow end of valve stent 506 and atrial clamp jaws 502 are released on the atrial side of native mitral valve annulus AN as shown in FIG. 29. Again, as discussed above, operator can pull/tug the removeable suture loops to adjust the placement of the valve if needed.

Once prosthetic mitral valve 501 is fully deployed at or adjacent to the native mitral valve annulus, as shown in FIG. 29, atrial clamp jaws 502 are seated on the atrial side of the native mitral valve annulus inside the left atrium, ventricular clamp jaws 503 are seated on the ventricular side of the native mitral valve annulus, and native mitral valve leaflets LF and annulus AN are clamped between atrial clamp jaws 502 and ventricular clamp jaws 503.

As shown in FIG. 29, in the deployed state of prosthetic mitral valve 501, ventricular clamp jaws 503 are on the ventricular side of the native mitral valve annulus whereas atrial clamp jaws 502 are on the atrial side of the native mitral valve annulus which means that in the deployed state of prosthetic mitral valve 501, atrial clamp jaws 502 are more distal from outflow end 506 than ventricular clamp jaws 503 are from outflow end 506. However, as discussed above, in the shape set configuration of prosthetic mitral valve 501, shown in FIGS. 22A and 22B, some parts of ventricular clamp jaws 503 are more distal from outflow end 506 than some parts of atrial clamp jaws 502 are from outflow end 506. Therefore, upon deployment of prosthetic mitral valve 501 at or adjacent to the native mitral valve annulus, ventricular clamp jaws 503 and atrial clamp jaws 502 are deformed from their shape set configurations relative to each other are resiliently biased. As discussed before, this deformation results in a resilient force between these two sets of jaws to try to restore their shape set configurations, the states shown in FIGS. 22A and 22B. This force acts to pinch or clamp the native mitral valve leaflets LF along with the native mitral valve annulus between atrial clamp jaws 502 and ventricular clamp jaws 503, thereby forming a secure anchor for prosthetic mitral valve 501 around the native mitral valve annulus.

At this point, in the embodiment shown in FIG. 29, leaflets LF and the native mitral valve annulus are pinched or clamped between atrial clamp jaws 502 and ventricular clamp jaws 503 and almost the entire mass of leaflets is substantially confined in a narrow region on the ventricular side the native mitral valve annulus. This narrow region can be defined by reference to distal annular point P (not shown), which is a point on the ventricular side of annulus AN which is located at the minimum longitudinal distance from the plane of outflow end 505 of stent 506. Longitudinal distance is the distance measured in a direction that is parallel to the longitudinal axis of stent 506. The narrow region can then be defined as the region bounded on one side by the ventricular side of annulus AN and on the other side by a plane that is perpendicular to the longitudinal axis of stent 506 and is located on the ventricular side of annulus AN at a longitudinal distance of W from point P. In some embodiments, W is less than 4 millimeters (4 mm). In other embodiments, W<3 mm, and yet other embodiments, W<2 mm. Therefore, in some embodiments of prosthetic mitral valve system, after deployment at or adjacent to the native mitral valve annulus, atrial and ventricular clamp jaws are separated by relatively short distance so that the structure formed by atrial and ventricular clamp jaws, 502 and 503, respectively, and native mitral valve leaflets LF and native annulus AN does not extend into or obstruct the LVOT. As discussed above, this is to be distinguished from prior art prosthetic mitral valve designs where some part or all of the native mitral valve leaflets are allowed to remain in a direction that is more or less parallel to the longitudinal axis of the prior art prosthetic mitral valve or dock. Such designs tend to increase obstruction of the left ventricle outflow tract (LVOT), rendering such designs unacceptable for a vast number of patients suffering from mitral valve regurgitation or other insufficiency.

Although the native mitral valve leaflets LF are pushed up to be held in the relatively narrow region bounded by atrial clamp jaws 502 and ventricular clamp jaws 503, leaflets LF continue to be attached to papillary muscles PM through tendinae chordae TC.

As discussed previously, the prosthetic mitral valve system in some embodiments can be delivered and deployed at or adjacent to the native mitral valve annulus using a retrograde approach to the mitral valve via the aorta and left ventricle.

In yet other embodiments, the prosthetic mitral valve system can be delivered and deployed at or adjacent to the native mitral valve annulus using a transapical approach. Thus, in an exemplary method, the prosthetic mitral valve is crimped under a sheath onto a custom-made 30F delivery device and then advanced through a 2-cm left atrial incision into the left ventricle (LV). Then, in a stepwise process, the atrial clamp jaws and ventricular clamp jaws of the prosthetic mitral valve are released so as to capture the native mitral valve leaflets. The sheath is then slowly withdrawn, releasing the valve stent. Once appropriate positioning is confirmed, the delivery device is removed.

### CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

What is claimed is:

1. A prosthetic mitral valve for implantation at a native mitral valve of a heart having a left atrium and a left ventricle, the native mitral valve having a native mitral valve annulus and native mitral valve leaflets, wherein the native mitral valve annulus has an atrium side that faces the left atrium of the heart and a ventricle side that faces the left ventricle of the heart, the prosthetic mitral valve system comprising:
an valve stent having an inflow end through which blood from the left atrium enters the prosthetic mitral valve and an outflow end out of which blood exits the prosthetic mitral valve to flow into the left ventricle;
one or more atrial clamp jaws projecting radially outwards from the valve stent;
one or more ventricular clamp jaws projecting radially outwards from the valve stent; and
a plurality of prosthetic leaflets coupled to the valve stent at commissure attachment features of the valve stent;
wherein when the prosthetic mitral valve is deployed at the site of the native mitral valve, the ventricular clamp jaws are deployed on the ventricle side of the native mitral valve annulus and atrial clamp jaws are deployed on the atrial side of the native mitral valve annulus such that the atrial clamp jaws and ventricular clamp jaws are sufficiently resiliently biased with respect to each other to grip the native mitral valve leaflets and the native mitral valve annulus between them.

2. The prosthetic mitral valve of claim 1, wherein the valve stent is comprised of a shape memory alloy.

3. The prosthetic mitral valve of claim 2, where the valve stent has a deployed configuration and a shape set configuration such that at least a portion of the ventricular clamp jaws is more distal to the outflow end in said shape set configuration than in the deployed configuration.

4. The prosthetic mitral valve of claim 1, wherein one or more ventricular clamp jaws is atraumatic.

5. The prosthetic mitral valve of claim 1, wherein one or more atrial clamp jaws is atraumatic.

6. The prosthetic mitral valve of claim 1, further comprising:
a fabric that covers the atrial clamp jaws.

7. The prosthetic mitral valve of claim 6, wherein the fabric covers one side of the atrial clamp jaws.

8. The prosthetic mitral valve of claim 1, further comprising:
a first removeable suture loop having free ends, wherein the first removeable suture loop is connected to a first one of the atrial clamp jaws.

9. The prosthetic mitral valve of claim 8, further comprising:
second and third removeable suture loops having free ends, wherein the second removeable suture loop is connected to a second one of the atrial clamp jaw and the third removeable suture loop is connected to a third one of the atrial clamp jaws.

10. The prosthetic mitral valve of claim 6, further comprising:
a circumferential suture that is connected to the atrial clamp jaws along the circumference of the prosthetic mitral valve.

11. The prosthetic mitral valve of claim 10, further comprising:
one or more removeable suture loops that are looped through the circumferential suture, each of the removeable suture loops having two free ends.

12. A prosthetic mitral valve for implantation at a native mitral valve of a heart having a left atrium and a left ventricle, the native mitral valve having a native mitral valve annulus and native mitral valve leaflets, wherein the native mitral valve annulus has an atrium side that faces the left atrium of the heart and a ventricle side that faces the left ventricle of the heart, the prosthetic mitral valve system comprising:
an expandable atrial valve stent having an inflow end and an outside diameter;
one or more atrial clamp jaws projecting radially outwards from the expandable atrial valve stent;
an expandable ventricular valve stent having an outflow end and an inside diameter that is greater than the outside diameter of the expandable atrial valve stent;
one or more ventricular clamp jaws projecting radially outwards from the valve stent; and
a plurality of prosthetic leaflets coupled to the expandable atrial valve stent at commissure attachment features of the expandable atrial valve stent;
wherein the expandable atrial valve stent is inserted into the expandable ventricular valve stent and the expandable atrial valve stent and the expandable ventricular valve stent are connected to each other such that the one or more atrial clamp jaws and the one or more ventricular clamp jaws are resiliently biased towards each to form a spring like clamp.

13. The prosthetic mitral valve of claim 12, wherein the expandable atrial valve stent and the expandable ventricular valve stent are comprised of a shape memory material.

14. The prosthetic mitral valve of claim 13, wherein the expandable ventricular valve stent has a deployed configuration and a shape set configuration such that at least a portion of the ventricular clamp jaws is more distal to the outflow end in the shape set configuration than in the deployed configuration.

15. A method of implanting a prosthetic mitral valve at a native mitral valve of a patient's heart having a left atrium and a left ventricle, the native mitral valve having a native mitral valve annulus and native mitral valve leaflets, wherein the native mitral valve annulus has an atrium side that faces the left atrium of the heart and a ventricle side that faces the left ventricle of the heart, and wherein the prosthetic mitral valve comprises an valve stent having an inflow end and an outflow end, one or more atrial clamp jaws projecting radially outwards from the valve stent, one or more ventricular clamp jaws projecting radially outwards from the valve stent, and a plurality of prosthetic leaflets coupled to the valve stent at commissure attachment features of the valve stent, wherein when the prosthetic mitral valve is deployed at the site of the native mitral valve, the ventricular clamp jaws are deployed on the ventricle side of the native mitral valve annulus and atrial clamp jaws are deployed on the atrial side of the native mitral valve annulus such that the atrial clamp jaws and ventricular clamp jaws are sufficiently resiliently biased with respect to each other to grip the native mitral valve leaflets and the native mitral valve annulus between them, the method comprising:
taking a delivery catheter that has a sheath with a proximal part and a distal part;
crimping the prosthetic mitral valve under the sheath of the delivery catheter such that the one or more atrial clamp jaws and the one or more ventricular clamp jaws are crimped under the proximal part of the sheath of the delivery catheter and the outflow end of the valve stent is crimped under the distal part of the sheath of the delivery catheter;
introducing the delivery catheter into the patient's body through percutaneous access;
moving the delivery catheter through the patient's body until the distal end of the delivery catheter is inside the left atrium of the patient's heart;
pulling the sheath of the delivery catheter proximally to release the ventricular clamp jaws;
advancing the delivery catheter through the left atrium and into the left ventricle of the patient's heart until the one or more ventricular clamp jaws have been pushed distally far enough into the left ventricle to be clear of the distal edges of native mitral valve leaflets;
pulling the delivery catheter in a proximal direction until the one or more ventricular clamp jaws abuts the ventricular side of the native mitral valve annulus pushing the native mitral valve leaflets against the native mitral valve annulus;
pushing the distal part of the sheath of the delivery catheter to release the outflow end of the valve stent;
withdrawing the proximal part of the sheath of the delivery catheter to release the one or more atrial clamp jaws such that the one or more atrial clamp jaws lie completely on the atrium side of the native mitral valve annulus touching at least some portion of the atrium side of the native mitral valve annulus; and
removing the delivery catheter from the patient's body.

16. The method of claim 15, wherein the prosthetic mitral valve further comprises a circumferential suture connected to the one or more atrial clamp jaws and one or more removeable suture loops connected to the circumferential suture, wherein the removeable suture loops are threaded through the delivery catheter such that the free ends of the removeable suture loops exit through the proximal handle of the delivery catheter, the method further comprising:
tugging on the one or more removeable suture loops to adjust the placement of the prosthetic mitral valve.

17. The method of claim 15, wherein when the atrial clamp jaws are released on the atrial side of the native mitral valve annulus, the native mitral valve leaflets are confined to a region that is bounded on one side by the ventricular side of the native mitral valve annulus and on the other side by a plane the minimal longitudinal distance of which from the ventricular side of the native mitral valve annulus is less than 6 mm.

18. The method of claim 15, wherein when the atrial clamp jaws are released on the atrial side of the native mitral valve annulus, the native mitral valve leaflets are confined to a region that is bounded on one side by the ventricular side of the native mitral valve annulus and on the other side by a plane the minimal longitudinal distance of which from the ventricular side of the native mitral valve annulus is less than 4 mm.

19. The method of claim 16, wherein after the atrial clamp jaws are released on the atrial side of the native mitral valve annulus and the placement of the valve has been adjusted, the native mitral valve leaflets are confined to a region that is bounded on one side by the ventricular side of the native mitral valve annulus and on the other side by a plane the minimal longitudinal distance of which from the ventricular side of the native mitral valve annulus is less than 6 mm.

20. The method of claim 16, wherein after the atrial clamp jaws are released on the atrial side of the native mitral valve annulus and the placement of the valve has been adjusted, the native mitral valve leaflets are confined to a region that is bounded on one side by the ventricular side of the native mitral valve annulus and on the other side by a plane the minimal longitudinal distance of which from the ventricular side of the native mitral valve annulus is less than 4 mm.
